(19) United States
(12) Patent Application Publication
(10) Pub. No.: US 2021/0347889 A1
(43) Pub. Date: Nov. 11, 2021

(54) DOSING REGIMEN OF ANTI-LAG3 ANTIBODY AND COMBINATION THERAPY WITH ANTI-PD-1 ANTIBODY FOR TREATING CANCER

(71) Applicant: Merck Sharp & Dohme Corp., Rahway, NJ (US)

(72) Inventors: Anson Kunjachan Abraham, Furlong, PA (US); Elliot K. Chartash, Basking Ridge, NJ (US); Kenneth Emancipator, Bernardsville, NJ (US); Rosario Garrido, Wayne, NJ (US); Jane Anne Healy, Hoboken, NJ (US); Jonathan W. Juco, Ridgewood, NJ (US); Diane Levitan, Tenafly, NJ (US); Qing Zhao, Jersey City, NJ (US)

(73) Assignee: Merck Sharp & Dohme Corp., Rahway, NJ (US)

(21) Appl. No.: 17/289,810

(22) PCT Filed: Nov. 4, 2019
(86) PCT No.: PCT/US2019/059583

§ 371 Date: Apr. 29, 2021

Related U.S. Application Data
(60) Provisional application No. 62/755,756, filed on Nov. 5, 2018.

(51) Int. Cl. C07K 16/28; A61P 35/00

(52) U.S. Cl. CPC C07K 16/2803; C07K 16/2827; A61K 2039/54; A61P 35/00; C07K 16/2818

(57) ABSTRACT

The present invention relates to dosing regimens of an anti-LAG3 antibody useful for the treatment of cancer. In particular, the invention relates to the dosing regimen in a combination therapy which comprises administering an antibody of a Programmed Death 1 protein (PD-1) or Programmed Death Ligand 1 (PD-L1) and an antibody of Lymphocyte-Activation Gene 3 (LAG3). The invention also provides a method for treating cancer in a patient comprising administering to the patient an anti-LAG3 antibody and an anti-PD-1 antibody, wherein the tumor tissue section of the patient is PD-L 1 expression positive, and optionally LAG3 expression positive.

### FIELD OF THE INVENTION

The present invention relates to dosing regimens of an anti-LAG3 antibody useful for the treatment of cancer. In particular, the invention relates to the dosing regimen in a combination therapy which comprises administering an antibody of a Programmed Death 1 protein (PD-1) or Programmed Death Ligand 1 (PD-L1) and an antibody of Lymphocyte-Activation Gene 3 (LAG3). The invention also provides a method for treating cancer in a patient comprising administering to the patient an anti-LAG3 antibody and an anti-PD-1 antibody, wherein the tumor tissue section of the patient is PD-L1 expression positive, optionally, LAG3 expression positive.

### BACKGROUND OF THE INVENTION

PD-1 is recognized as an important molecule in immune regulation and the maintenance of peripheral tolerance. PD-1 is moderately expressed on naive T, B and NKT cells and up-regulated by TB cell receptor signaling on lymphocytes, monocytes and myeloid cells (1)

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (2-13). Similarly, PD-1 expression on tumor infiltrating lymphocytes was found to mark dysfunctional T cells in breast cancer and melanoma (14-15) and to correlate with poor prognosis in renal cancer (16). Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab is a potent humanized immunoglobulin G4 (IgG4) mAb with high specificity of binding to the programmed cell death 1 (PD 1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1 Keytruda® (pembrolizumab) is indicated for the treatment of patients across a number of indications.

Lymphocyte-Activation Gene 3 (LAG3) is an inhibitory immune modulatory receptor that regulates effector T cell homeostasis, proliferation, and activation, and has a role in the suppressor activity of regulatory T cells (Tregs). LAG3 is expressed on activated CD8+ and CD4+ T cells, Tregs and the Trl regulatory T-cell population, as well as on natural killer cells and a subset of tolerogenic plasmacytoid dendritic cells. Because of its proposed role on both effector T cells and Tregs, LAG3 is one of several immune checkpoint molecules where simultaneous blockade of both cell populations has the potential to enhance antitumor immunity.

LAG3 is structurally related to cluster of differentiation (CD) 4 and a member of the immunoglobulin (Ig) superfamily. Like CD4, its ligand is major histocompatibility complex (MHC) Class II molecules. Interaction with its ligand leads to dimerization and signal transduction resulting in altered T-cell activation. Following T-cell activation, LAG3 is transiently expressed on the cell surface. A large proportion of LAG3 molecules are found in intracellular stores and can be rapidly translocated to the cell membrane upon T-cell activation. LAG3 expression is regulated at the cell surface by extracellular cleavage to yield a soluble form of LAG3 (sLAG 3), which can be detected in serum. Expression of LAG3 is tightly regulated and represents a self-limiting mechanism to counter uncontrolled T-cell activity. Anti-LAG3 antibodies have been described in WO2016/028672.

Selecting a dosage regimen for an anti-LAG3 antibody monotherapy or combination therapy with anti-PD-1 or anti-PD-L1 therapy depends on several factors, including the serum or tissue turnover rate of the entity, the level of symptoms, the immunogenicity of the entity, antidrug antibody endpoints and the accessibility of the target cells, tissue or organ in the individual being treated, as well as safety. Formation of antidrug antibodies can potentially confound drug exposures at therapeutic doses, and prime for subsequent infusion-related toxicities. In addition, anti-LAG3 and/or anti-PD-1/anti-PD-L1 treatment can result in immune stimulation and the potential for cytokine release that affects safety.

### SUMMARY OF THE INVENTION

The invention provides a method for treating cancer in a patient comprising administering 7-1200 mg of an anti-LAG3 antibody Ab6. In one embodiment, 200-800 mg of an anti-LAG3 antibody Ab6 is administered. In another embodiment, 800 mg of an anti-LAG3 antibody Ab6 is administered. In one embodiment, the method optionally comprises co-administration with an anti-PD-1 or anti-PD-L1 antibody. In one embodiment, the anti-LAG3 antibody and anti-PD-1 antibody are co-formulated. In another embodiment, the tumor tissue section of the patient is PD-L1 expression positive. In a further embodiment, the tumor cells of the patient is PD-L1 expression positive. In one embodiment, the anti-PD-1 antibody blocks the binding of PD-1 to PD-L1 and PD-L2. The invention also provides a pharmaceutical composition comprising 7-1200 mg of anti-LAG3 antibody Ab6 or Ab6 variant, and 200 mg of pembrolizumab or pembrolizumab variant. In one embodiment, the pharmaceutical composition comprises 800 mg of anti-LAG3 antibody Ab6 or Ab6 variant, and 200 mg of pembrolizumab or pembrolizumab variant.

The invention also provides a method for treating non-MSI-H colorectal cancer, gastric cancer or head and neck squamous cell carcinoma in a patient comprising administering to the patient an anti-LAG3 antibody and an anti-PD-1 antibody, wherein the tumor tissue section of the patient is PD-L1 expression positive, and optionally LAG3 expression positive.

### BRIEF DESCRIPTION OF THE DRAWINGS

FIG. 1 CT scan of patient with non-MSI-H colorectal cancer before (left) and after (right) treatment with 21 mg anti-LAG3 antibody Ab6 and pembrolizumab. The patient received 5 prior lines of chemotherapy, no prior anti-PD-1 or anti-PD-L1 therapy. The patient had a partial response with 45% reduction in tumor volume. There was also tumor volume reduction in lung lesions and lymph nodes, and stable presacral mass. The response is ongoing at 13.5 months.

FIG. 2 CT scan of a 60-year-old male with renal cell carcinoma and metastases to lung and bone before (left) and after (right) treatment with 7 mg anti-LAG3 antibody Ab6 and pembrolizumab. The patient received 3 prior lines of therapy, including prior anti-PD-1 therapy. The patient had a partial response at 9 weeks with 49% reduction in tumor volume. Tumor volume reduction was observed at all visible disease sites including the lung and multiple lymph nodes. The response lasted for 15 months before disease progression.

FIG. 3 Waterfall plot of subjects with best target lesion change from baseline based on investigator assessment per RECIST 1.1 FAS population in the colorectal cancer expansion cohort (Part B) using the PD-L1 IHC Combined Positive score (CPS). Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. Tumor samples with CPS>=1 or <1 are indicated. Tumor samples with less than 100 tumor cells cannot be interpreted.

FIG. 4 Waterfall plot of subjects with best target lesion change from baseline based on investigator assessment per RECIST 1.1 FAS population in the colorectal cancer expansion cohort (Part B) using the LAG3 IHC CPS-like LAG3 positive cells scoring system.

Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. Tumor samples with CPS>=1 or <1 are indicated. Tumor samples with less than 100 tumor cells cannot be interpreted.

FIG. 5 Serum concentrations of Ab6 following intravenous doses from 7 mg to 700 mg in cycle 1, Part A of the phase I study. Arithmetic mean serum concentration for each dose is plotted at nominal times.

FIG. 6 Serum concentrations of total soluble LAG-3 following intravenous doses from 7 mg to 700 mg in cycle 1, Part A of the phase I study. Arithmetic mean of total soluble LAG-3 plotted at nominal times.

FIG. 7A-B shows that pembrolizumab Cmax at steady state for 400 mg Q6W lies within the range from 2 mg/kg and 200 mg Q3W to 10 mg/kg Q2W. 7A: pembrolizumab Cmax at steady state for 2 mg/kg and 200 mg Q3W. 7B: pembrolizumab Cmax at steady state for 400 mg Q6W and 10 mg/kg Q2W.

FIG. 8 shows that pembrolizumab exposures (Cavg and Cmin) at steady state are similar for 400 mg Q6W relative to 2 mg/kg Q3W and 200 mg Q3W.

FIG. 9A-B shows the pembrolizumab pharmacokinetic profiles at steady state for the 400 mg Q6W dosing regimen compared to the Q3W, 200 mg flat dosing regimen (top) and the Q3W, 2 mg/kg weight-based dosing regimen (bottom). 9A shows the log scale concentrations, and 9B shows the linear scale concentrations.

FIG. 10 Serum concentrations of Ab6 following intravenous doses from 7 mg to 700 mg in cycle 1 on linear scale with additional patient sampling compared to FIG. 5. The arithmetic mean of Ab6 serum concentrations is plotted at nominal times.

FIG. 11 Serum concentrations of Ab6 following intravenous doses from 7 mg to 700 mg in cycle 1 on log scale with additional patient sampling compared to FIG. 5. The arithmetic mean of Ab6 serum concentrations is plotted at nominal times.

FIG. 12 Serum concentrations of total soluble LAG3 following intravenous doses from 7 mg to 700 mg in cycle 1 with additional patient sampling compared to FIG. 6. The arithmetic mean of total soluble LAG3 serum concentrations is plotted at nominal times.

FIG. 13 Predicted Ab6 serum concentration-time profiles in Cycle 1 corresponding to the 800 mg dose overlaid with observed concentrations for the 700 mg dose. Solid markers represent observed Ab6 serum concentrations at 700 mg from the Phase I study. Shaded areas represent 2.5th and 97.5th percentiles for predicted concentrations for the 800 mg dose. Ab6 exposures from cycle 1 are expected to be representative of subsequent treatment cycles.

FIG. 14 Predicted Ab6 exposures (AUC, Ctrough, Cmax) as a function of dose showing substantial overlapping exposures between the 700 mg and 800 mg doses. Straight lines: median; box: 25th and 75th percentile, whiskers: 5th and 95th percentiles.

FIG. 15 Box-plot of Ab6 serum Ctrough on Day 21 showing PK variability.

FIG. 16 Waterfall plot of subjects with best target lesion change from baseline based on investigator assessment per RECIST 1.1 FAS population in the gastric cancer expansion cohort (Part B) using the PD-L1 IHC Combined Positive score (CPS).

Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. Tumor samples with CPS >=1 or <1 are indicated. Tumor samples with less than 100 tumor cells cannot be interpreted.

FIG. 17 Waterfall plot of subjects with best target lesion change from baseline based on investigator assessment per RECIST 1.1 FAS population in the gastric cancer expansion cohort (Part B) using the LAG3 IHC CPS-like % LAG3 positive cells scoring method.

Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. Tumor samples with CPS >=1 or <1 are indicated. Tumor samples with less than 100 tumor cells cannot be interpreted.

FIG. 18 Waterfall plot of subjects with best target lesion change from baseline based on investigator assessment per RECIST 1.1 FAS population in the HSNCC PD-L1 naive cancer expansion cohort (Part B) using the PD-L1 IHC TPS+MIDS scoring system.

Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. Tumor samples with CPS>=1 or <1 are indicated. Tumor samples with less than 100 tumor cells cannot be interpreted.

FIG. 19 Waterfall plot of subjects with best target lesion change from baseline based on investigator assessment per RECIST 1.1 FAS population in the HSNCC PD-L1 naïve cancer expansion cohort (Part B) using the LAG3 IHC % LAG3 positive cells scoring system. Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. Tumor samples with CPS>=1 or <1 are indicated. Tumor samples with less than 100 tumor cells cannot be interpreted.

### DETAILED DESCRIPTION

Abbreviations. Throughout the detailed description and examples of the invention the following abbreviations will be used:
BOR Best overall response
BID One dose twice daily
CBR Clinical Benefit Rate
CDR Complementarity determining region
CHO Chinese hamster ovary
CR Complete Response
DCR Disease Control Rate
DFS Disease free survival
DLT Dose limiting toxicity
DOR Duration of Response
DSDR Durable Stable Disease Rate
FFPE Formalin-fixed, paraffin-embedded
FR Framework region
IgG Immunoglobulin G
IHC Immunohistochemistry or immunohistochemical
irRC Immune related response criteria
IV Intravenous
MTD Maximum tolerated dose
NCBI National Center for Biotechnology Information
NCI National Cancer Institute
ORR Objective response rate
OS Overall survival
PD Progressive disease
PD-1 Programmed Death 1
PD-L1 Programmed Cell Death 1 Ligand 1
PD-L2 Programmed Cell Death 1 Ligand 2
PFS Progression free survival
PR Partial response
Q2W One dose every two weeks
Q3W One dose every three weeks
QD One dose per day
RECIST Response Evaluation Criteria in Solid Tumors
SD Stable disease
VH Immunoglobulin heavy chain variable region
VK Immunoglobulin kappa light chain variable region

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, an “Ab6 variant” means a monoclonal antibody which comprises heavy chain and light chain sequences that are substantially identical to those in Ab6 (as described below and in WO2016028672, incorporated by reference in its entirety), except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR regions or the constant region, and optionally has a deletion of the C-terminal lysine residue of the heavy chain. In other words, Ab6 and a Ab6 variant comprise identical CDR sequences, but differ from each other due to having a conservative amino acid substitution at no more than three or six other positions in their full length light and heavy chain sequences, respectively. An Ab6 variant is substantially the same as Ab6 with respect to the following properties: binding affinity to human LAG3 and ability to block the binding of human LAG3 to human WIC Class II.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “subject” includes any organism, preferably an animal, more preferably a mammal (e.g., rat, mouse, dog, cat, rabbit) and most preferably a human.

As used herein, the term “antibody” refers to any foul' of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of

Health, Bethesda, Md. ; 5t^{h} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Co-administration” as used herein for agents such as the PD-1 antagonist or LAG3 antagonist means that the agents are administered so as to have overlapping therapeutic activities, and not necessarily that the agents are administered simultaneously to the subject. The agents may or may not be in physical combination prior to administration. In an embodiment, the agents are administered to a subject simultaneously or at about the same time. For example, the anti-PD-1 antibody and anti-LAG3 drug products contained in separate vials, when in liquid solution, may be mixed into the same intravenous infusion bag or injection device, and administered simultaneously to the patient.

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

Pharmacokinetic “steady state” is a period of time during which any accumulation of drug concentrations owing to multiple doses has been maximized and systemic drug exposure is considered uniform after each subsequent dose administered; in the specific case of pembrolizumab, steady state is achieved at and after ˜16 weeks of administration.

AUCss, Cavg,ss and Cmin,ss are pharmacokinetic measures of the systemic exposure to the drug (e.g. pembrolizumab) in humans after its administration, and are typically considered drivers of drug efficacy. AUCss and Cavg,ss represent the average exposure over a dosing interval, but differ in terms of units. “Cmin,ss” represents the minimum or lowest (trough) drug concentration observed at the end of a dosing interval, just before the next dose is administered.

“Cmax,ss” is the maximum or highest (peak) drug concentration observed soon after its administration. In the specific case of pembrolizumab, which is administered as intravenous infusion, the peak concentration occurs immediately after end of infusion. Cmax,ss is a metric that is typically considered a driver of safety.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic regimen, such as a combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a combination therapy described herein is assessed using RECIST 1.1 criteria, bidimentional irRC or unidimensional irRC. In some embodiments, an anti-tumor response is any of SD, PR, CR, PFS, or DFS.

“Bidimensional irRC” refers to the set of criteria described in Wolchok JD, et al. Guidelines for the evaluation of immune therapy activity in solid tumors: immune-related response criteria. Clin Cancer Res. 2009;15(23):7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand / receptor signaling in any biological pathway that supports tumor maintenance and/or growth or suppresses the anti-tumor immune response. Classes of biotherapeutic agents include, but are not limited to, antibodies to VEGF, EGFR, Her2/neu, other growth factor receptors, CD20, CD40, CD-40L, CTLA-4, OX-40, 4-1BB, and ICOS.

“CBR” or “Clinical Benefit Rate” means CR+PR+durable SD.

“CDR” or “CDRs” as used herein means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in Al-Lazikani et aL , JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“DCR” or “Disease Control Rate” means CR+PR+SD.

“Diagnostic anti-PD-L monoclonal antibody” means a mAb which specifically binds to the mature form of the designated PD-L (PD-L1 or PDL2) that is expressed on the surface of certain mammalian cells. A mature PD-L lacks the presecretory leader sequence, also referred to as leader peptide. The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 32)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRHMDVKKCGIQDTNSKKQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO2014/100079. Another anti-human PD-L1 mAb that has been reported to be useful for IHC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P. R. China; Catalog number 10084-R015).

TABLE 2
Characteristics of Monoclonal Antibody
MEB037.22C3 (22C3)

| | | |
|---|---|---|
| | | SEQ |
| Antibody | | ID |
| Feature | Amino Acid Sequence | NO |

Light Chain

| | | |
|---|---|---|
| CDRL1 | KSSQSLLHTSTRKNYLA | 13 |
| CDRL2 | WASTRES | 14 |
| CDRL3 | KQSYDVVT | 15 |
| Mature | DIVMSQSPSSLAVSAGEKVTMTCKSSQSLLHTSTRKNY | 16 |
| Variable | LAWYQQKPGQSPKLLIYWASTRESGVPDRFTGSGSGTD | |
| Region | FTLTISSVQAEDLAVYYCKQSYDVVTFGAGTKLELK | |

Heavy Chain

| | | |
|---|---|---|
| CDRH1 | SYWIH | 17 |
| Kabat | | |
| Def'n | | |
| CDRH1 | GYTFTSYWIH | 18 |
| Chothia | | |
| Def'n | | |
| CDRH2 | YINPSSGYHEYNQKFID | 19 |
| CDRH3 | SGWLIHGDYYFDF | 20 |
| Mature | XVHLQQSGAELAKPGASVKMSCKASGYTFTSYWIHWIK | 21 |
| Variable | QRPGQGLEWIGYINPSSGYHEYNQKFIDKATLTADRSS | |
| Region | STAYMHLTSLTSEDSAVYYCARSGWLIHGDYYFDFWGQ | |
| | GTTLTVSS, | |
| | wherein X = Q or pE (pyro-glutamate) | |

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR, realtime quantitative RT-PCR, RNAseq, and the Nanostring platform (J. Clin. Invest. 2017;127(8):2930-2940).

Several approaches have been described for quantifying PD-L1 protein expression in IHC assays of tumor tissue sections. See, e.g., Thompson, R. H., et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Toplian, S. L. et al., New Eng. JMed. 366 (26): 2443-2454 (2012). See US 20170285037 which describes Hematoxylin and Eosin staining used by the pathologist.

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression if it is at least 1% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

The level of PD-L mRNA expression may be compared to the mRNA expression levels of one or more reference genes that are frequently used in quantitative RT-PCR.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/ or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

“Tumor proportion score (TPS)” refers to the percentage of tumor cells expressing PD-L1 on the cell membrane at any intensity (weak, moderate or strong). Linear partial or complete cell membrane staining is interpreted as positive for PD-L1.

“Mononuclear inflammatory density score (MIDS)” refers to the ratio of the number of PD-L1 expressing mononuclear inflammatory cells (MIC) infiltrating or adjacent to the tumor (small and large lymphocytes, monocytes, and macrophages within the tumor nests and the adjacent supporting stroma) compared to the total number of tumor cells. The MIDS is recorded at a scale from 0 to 4 with 0=none; 1=present, but less than one MIC for every 100 tumor cells (<1%); 2=at least one MIC for every 100 tumor cells, but less than one MIC per 10 tumor cells (1-9%); 3=at least one MIC for every 10 tumor cells, but fewer MIC's than tumor cells (10-99%); 4=at least as many MIC's as tumor cells (≥100%).

“Combined positive score (CPS)” refers to the ratio of the number of PD-L1 positive tumor cells and PD-L1 positive mononuclear inflammatory cells (MIC) within the tumor nests and the adjacent supporting stroma (numerator) compared to the total number of tumor cells (denominator; i.e., the number of PD-L1 positive and PD-L1 negative tumor cells). PD-L1 expression at any intensity is considered positive, i.e., weak (1+), moderate (2+), or strong (3+).

“PD-L1 expression positive” refers to a Tumor Proportion Score, Mononuclear Inflammatory Density Score or Combined Positive Score of at least 1%; AIS is ≥5; or elevated level of PD-L1 expression (protein and/or mRNA) by malignant cells and/or by infiltrating immune cells within a tumor compared to an appropriate control.

LAG3 protein expression may be detected with a diagnostic anti-LAG3 antibody in an IHC assay of a tumor tissue section or by flow cytometry. In one embodiment, the diagnostic anti-LAG3 antibody is clone 17B4 from LSBio. Alternatively, LAG3 protein expression by tumor cells may be detected by PET imaging, using a binding agent (e.g., antibody fragment, affibody and the like) that specifically binds to LAG3. Techniques for detecting and measuring LAG3 mRNA expression include RT-PCR, realtime quantitative RT-PCR, RNAseq, and the Nanostring platform (J. Clin. Invest. 2017;127(8):2930-2940).

“% LAG3 positive cells” refers to LAG3 positive cells/all cells in tumor area x100 Linear partial or complete immune cell membrane staining in an IHC assay is interpreted as positive for LAG3

“CPS-like % LAG3 positive cells” refers to LAG3 positive cells/tumor cells in tumor area x100. Linear partial or complete immune cell membrane staining in an IHC assay is interpreted as positive for LAG3.

“LAG3 expression positive” refers to the % LAG3 positive cells or CPS-like % LAG3 positive cells≥1%

“DSDR” or “Durable Stable Disease Rate” means SD for ≥23 weeks.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Anti-LAG3 antibody” means a monoclonal antibody that blocks binding of LAG3 expressed on an immune cell (T cell, Tregs, or NK cell etc.) to MHC Class II molecules. Human LAG3 comprises the amino acid sequence:

(SEQ ID NO: 33)
MWEAQFLGLL FLQPLWVAPV KPLQPGAEVP VVWAQEGAPA
QLPCSPTIPL QDLSLLRRAG VTWQHQPDSG PPAAAPGHPL
APGPHPAAPS SWGPRPRRYT VLSVGPGGLR SGRLPLQPRV
QLDERGRQRG DFSLWLRPAR RADAGEYRAA VHLRDRALSC
RLRLRLGQAS MTASPPGSLR ASDWVILNCS FSRPDRPASV
HWFRNRGQGR VPVRESPHHH LAESFLFLPQ VSPMDSGPWG
CILTYRDGFN VSIMYNLTVL GLEPPTPLTV YAGAGSRVGL
PCRLPAGVGT RSFLTAKWTP PGGGPDLLVT GDNGDFTLRL
EDVSQAQAGT YTCHIHLQEQ QLNATVTLAI ITVTPKSFGS
PGSLGKLLCE VTPVSGQERF VWSSLDTPSQ RSFSGPWLEA
QEAQLLSQPW QCQLYQGERL LGAAVYFTEL SSPGAQRSGR
APGALPAGHL LLFLILGVLS LLLLVTGAFG FHLWRRQWRP
RRFSALEQGI HPPQAQSKIE ELEQEPEPEP EPEPEPEPEP EPEQL;
see also Uniprot accession no. P18627.

“Microsatellite instability (MSI)” refers to the form of genomic instability associated with defective DNA mismatch repair in tumors. See Boland et al., Cancer Research 58, 5258-5257, 1998. In one embodiment, MSI analysis can be carried out using the five National Cancer Institute (NCI) recommended microsatellite markers BAT25 (GenBank accession no. 9834508), BAT26 (GenBank accession no. 9834505), D5S346 (GenBank accession no. 181171), D2S123 (GenBank accession no. 187953), D17S250 (GenBank accession no. 177030). Additional markers for example, BAT40, BAT34C4, TGF-β-RII and ACTC can be used. Commercially available kits for MSI analysis include, for example, the Promega MSI multiplex PCR assay.

“High frequency microsatellite instability” or “microsatellite instability-high (MSI-H)” refers to if two or more of the five NCI markers show instability or ≥30-40% of the total markers demonstrate instability (i.e. have insertion/deletion mutations).

“Low frequency microsatellite instability” or “microsatellite instability-low (MSI-L)” refers to if one of the five NCI markers show instability or <30-40% of the total markers exhibit instability (i.e. have insertion/deletion mutations).

“Non-MSI-H colorectal cancer” as used herein refers to microsatellite stable (MSS) and low frequency MSI (MSI-L) colorectal cancer.

“Microsatellite Stable (MSS)” refers to if none of the five NCI markers show instability (i.e. have insertion/deletion mutations)

“Proficient mismatch repair (pMMR) colorectal cancel” refers to normal expression of MMR proteins (MLH1, PMS2, MSH2, and MSH6) in a CRC tumor specimen by IHC. Commercially available kits for MMR analysis include theVentana MMR IHC assay.

“Mismatch repair deficient (dMMR) colorectal cancer” refers to low expression of one or more MMR protein(s) (MLH1, PMS2, MSH2, and MSH6) in a CRC tumor specimen by IHC.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Non-responder patient”, when referring to a specific anti-tumor response to treatment with a combination therapy described herein, means the patient did not exhibit the anti-tumor response.

“ORR” or “objective response rate” refers in some embodiments to CR+PR, and ORR(week 24) refers to CR and PR measured using irRECIST in each patient in a cohort after 24 weeks of anti-cancer treatment .

“Patient” or “subject” refers to any single subject for which therapy is desired or that is participating in a clinical trial, epidemiological study or used as a control, including humans and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“RECIST 1.1 Response Criteria” as used herein means the definitions set forth in Eisenhauer et al., E.A. et al., Eur. J Cancer 45:228-247 (2009) for target lesions or nontarget lesions, as appropriate based on the context in which response is being measured.

“Responder patient” when referring to a specific anti-tumor response to treatment with a combination therapy described herein, means the patient exhibited the anti-tumor response.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” cancer as used herein means to administer therapeutic agents of the invention to a subject having cancer, or diagnosed with cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, response to a combination therapy described herein is assessed using RECIST 1.1 criteria or irRC (bidimensional or unidimensional) and the treatment achieved by a combination of the invention is any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination of the invention is any of PR, CR, PFS, DFS, OR and OS that is assessed using RECIST 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi^{t}-test, the U-test according to Mann and Whitney, the Kruskal-Wallis test (H-test), Jonckheere-Terpstra-test and the Wilcoxon-test.

The terms “treatment regimen”, “dosing protocol” and “dosing regimen” are used interchangeably to refer to the dose and timing of administration of each therapeutic agent in a combination of the invention.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC refers to the set of criteria described in Nishino M, Giobbie-Hurder A, Gargano M, Suda M, Ramaiya NH, Hodi FS. Developing a Common Language for Tumor Response to Immunotherapy: Immune-related Response Criteria using Unidimensional measurements. Clin Cancer Res. 2013;19(14):3936-3943). These criteria utilize the longest diameter (cm) of each lesion.

“Variable regions” or “V region” as used herein means the segment of IgG chains which is variable in sequence between different antibodies. Typically, it extends to Kabat residue 109 in the light chain and 113 in the heavy chain.

### PD-1 Antagonists and ANTI-LAG3 Antibodies

PD-1 antagonists useful in the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments. The anti-PD-1 or anti-PD-L1 antibody may be produced in CHO cells using conventional cell culture and recovery/purification technologies.

Examples of mAbs that bind to human PD-1, and useful in the treatment method, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in Table 3; nivolumab (BMS-936558), a human IgG4 mAb with the structure described in WHO Drug Information, Vol. 27, No. 1, pages 68-69 (2013) and which comprises the heavy and light chain amino acid sequences shown in Table 3; the humanized antibodies h409A11, h409A16 and h409A17, which are described in WO2008/156712, and AMP-514, which is being developed by MedImmune.

Examples of mAbs that bind to human PD-L1, and useful in the treatment method, medicaments and uses of the present invention, are described in WO2013/019906, WO2010/077634 A1 and US8383796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in the treatment method, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

In some preferred embodiments of the treatment method, medicaments and uses of the present invention, the PD-1 antagonist is a monoclonal antibody, or antigen binding fragment thereof, which comprises: (a) light chain CDRs SEQ ID NOs: 1, 2 and 3 and (b) heavy chain CDRs SEQ ID NOs: 6, 7 and 8.

In other preferred embodiments of the treatment method, medicaments and uses of the present invention, the PD-1 antagonist is a monoclonal antibody, or antigen binding fragment thereof, which specifically binds to human PD-1 and comprises (a) a heavy chain variable region comprising SEQ ID NO:9 or a variant thereof, and (b) a light chain variable region comprising SEQ ID NO:4 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the PD-1 antagonist is a monoclonal antibody which specifically binds to human PD-1 and comprises (a) a heavy chain comprising SEQ ID NO: 10 and (b) a light chain comprising SEQ ID NO:5.

In yet another preferred embodiment of the treatment method, medicaments and uses of the present invention, the PD-1 antagonist is a monoclonal antibody which specifically binds to human PD-1 and comprises (a) a heavy chain comprising SEQ ID NO: 12 and (b) a light chain comprising SEQ ID NO:11.

In all of the above treatment method, medicaments and uses, the PD-1 antagonist inhibits the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain, and wherein the heavy and light chains comprise the amino acid sequences in SEQ ID NO:10 and SEQ ID NO:5, respectively.

Table 3 below provides a list of the amino acid sequences of exemplary anti-PD-1 mAbs for use in the treatment method, medicaments and uses of the present invention.

TABLE 3
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 4 |
| Region | QQKPGQAPRLLIYLASYLESGVPARFSGSGSGTDFTLTISS | |
| | LEPEDFAVYYCQHSRDLPLTFGGGTKVEIK | |
| Light Chain | EIVLTQSPATLSLSPGERATLSCRASKGVSTSGYSYLHWY | 5 |
| | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQS | |
| | GNSQESVTEQDSKDSTYSLSSTLTLSKADYEKHKVYACE | |
| | VTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWV | 9 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYFP | |
| | EPVTVSWNSGALTSGVHTFPAVLQSSGLYSLSSVVTVPSS | |
| | SLGTKTYTCNVDHKPSNTKVDKRVESKYGPPCPPCPAPE | |
| | FLGGPSVFLFPPKPKDTLMISRTPEVTCVVVDVSQEDPEV | |
| | QFNWYVDGVEVHNAKTKPREEQFNSTYRVVSVLTVLHQ | |
| | DWLNGKEYKCKVSNKGLPSSIEKTISKAKGQPREPQVYT | |
| | LPPSQEEMTKNQVSLTCLVKGFYPSDIAVEWESNGQPEN | |
| | NYKTTPPVLDSDGSFFLYSRLTVDKSRWQEGNVFSCSVM | |
| | HEALHNHYTQKSLSLSLGK | |

Nivolumab Light Chain

| | | |
|---|---|---|
| Light Chain | EIVLTQSPATLSLSPGERATLSCRASQSVSSYLAWYQQKP | 11 |
| | GQAPRLLIYDASNRATGIPARFSGSGSGTDFTLTISSLEPE | |
| | DFAVYYCQQSSNWPRTFGQGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |

Nivolumab Heavy Chain

| | | |
|---|---|---|
| Heavy | QVQLVESGGGVVQPGRSLRLDCKASGITFSNSGMEIWVR | 12 |
| Chain | QAPGKGLEWVAVIWYDGSKRYYADSVKGRFTISRDNSK | |
| | NTLFLQMNSLRAEDTAVYYCATNDDWGQGTLVTVSSA | |
| | STKGPSVFPLAPCSRSTSESTAALGCLVKDYFPEPVTVSW | |
| | NSGALTSGVHTFPAVLQSSGLYSLSSVVTVPSSSLGTKTY | |
| | TCNVDHKPSNTKVDKRVESKYGPPCPPCPAPEFLGGPSVF | |
| | LFPPKPKDTLMISRTPEVTCVVVDVSQEDPEVQFNWYVD | |
| | GVEVHNAKTKPREEQFNSTYRVVSVLTVLHQDWLNGKE | |
| | YKCKVSNKGLPSSIEKTISKAKGQPREPQVYTLPPSQEEM | |
| | TKNQVSLTCLVKGFYPSDIAVEWESNGQPENNYKTTPPV | |
| | LDSDGSFFLYSRLTVDKSRWQEGNVFSCSVMHEALHNH | |
| | YTQKSLSLSLGK | |

The anti-LAG3 antibody used in the claimed invention may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region.

In one embodiment, the anti-LAG3 antibody is Ab6.

Ab6: a light chain immunoglobulin comprising the amino acid sequence:

(SEQ ID NO: 22)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC;

and a heavy chain immunoglobulin comprising the amino acid sequence:

(SEQ ID NO: 23)
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;

or a light chain immunoglobulin variable domain comprising the amino acid sequence:

(SEQ ID NO: 24 (CDRs underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;

and a heavy chain immunoglobulin variable domain comprising the amino acid sequence:

(SEQ ID NO: 25 (CDRs underscored))
QMQLVQSGPEVKKPGTSVKVSCKASGYTFTDYNVDWVRQARGQRLEWIG
DINPNDGGTIYAQKFQERVTITVDKSTSTAYMELSSLRSEDTAVYYCAR
NYRWFGAMDHWGQGTTVTVSS;

or; comprising the CDRs:

(SEQ ID NO: 26)
CDR-L1: KASQSLDYEGDSDMN;
(SEQ ID NO: 27)
CDR-L2: GASNLES;
(SEQ ID NO: 28)
CDR-L3: QQSTEDPRT;
(SEQ ID NO: 29)
CDR-H1: DYNVD;
(SEQ ID NO: 30)
CDR-H2: DINPNDGGTIYAQKFQE;
and
(SEQ ID NO: 31)
CDR-H3: NYRWFGAMDH

In some preferred embodiments of the treatment method, medicaments and uses of the present invention, the anti-LAG3 antibody comprises: (a) light chain CDRs SEQ ID NOs: 26, 27 and 28 and (b) heavy chain CDRs SEQ ID NOs: 29, 30 and 31.

In other preferred embodiments of the treatment method, medicaments and uses of the present invention, the anti-LAG3 antibody comprises (a) a heavy chain variable region comprising SEQ ID NO:25 or a variant thereof, and (b) a light chain variable region comprising SEQ ID NO:24 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the anti-LAG3 antibody comprises (a) a heavy chain comprising SEQ ID NO: 23 and (b) a light chain comprising SEQ ID NO:22. In another preferred embodiment of the treatment method, medicaments and uses of the present invention, the anti-LAG3 antibody comprises (a) a heavy chain variable region comprising SEQ ID NO: 25 and (b) a light chain variable region comprising SEQ ID NO:24.

In one embodiment, the anti-PD-1 or anti-LAG3 antibody or antigen-binding fragment comprises a heavy chain constant region, e.g. a human constant region, such as γ1, γ2, γ3, or γ4 human heavy chain constant region or a variant thereof. In another embodiment, the anti-PD-1 or anti-LAG3 antibody or antigen-binding fragment comprises a light chain constant region, e.g. a human light chain constant region, such as lambda or kappa human light chain region or variant thereof. By way of example, and not limitation, the human heavy chain constant region can be y4 and the human light chain constant region can be kappa. In an alternative embodiment, the Fc region of the antibody is γ4 with a Ser228Pro mutation (Schuurman, J et. al., Mol. Immunol. 38: 1-8, 2001).

In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. For example, a human IgG4 constant domain, for example, may be used. The present invention includes the use of anti-PD-1 antibodies or anti-LAG3 antibodies and antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105.

### Methods, Uses and Medicaments

In one aspect, the invention provides a method of treating cancer in a patient comprising administering an anti-LAG3 antibody at 7-1200 mg via intravenous infusion, wherein the anti-LAG3 antibody comprises: (a) light chain CDRs of SEQ ID NOs: 26, 27 and 28 and (b) heavy chain CDRs of SEQ ID NOs: 29, 30 and 31. In another aspect, the invention provides a method of treating cancer in a patient comprising co-administering an anti-LAG3 antibody at 7-1200 mg via intravenous infusion with an anti-PD-1 or anti-PD-L1 antibody, wherein the anti-LAG3 antibody comprises: (a) light chain CDRs of SEQ ID NOs: 26, 27 and 28 and (b) heavy chain CDRs of SEQ ID NOs: 29, 30 and 31. In one embodiment, the anti-PD-1 antibody blocks the binding of PD-1 to PD-L1 and PD-L2. In one embodiment, 7-800 mg of the anti-LAG3 antibody is administered. In another embodiment, 100-800 mg of the anti-LAG3 antibody is administered. In another embodiment, 200 mg of the anti-LAG3 antibody is administered. In another embodiment, 700 mg of the anti-LAG3 antibody is administered. In another embodiment, 800 mg of the anti-LAG3 antibody is administered. In another embodiment, 200-800 mg of the anti-LAG3 antibody is administered. In another embodiment, 200-700 mg of the anti-LAG3 antibody is administered. In another embodiment, 200-700 mg of the anti-LAG3 antibody is administered. In a further embodiment, 200-900 mg of the anti-LAG3 antibody is administered. In a further embodiment, 200-1000 mg of the anti-LAG3 antibody is administered.

In a further aspect, the invention provides a method for treating cancer in a patient comprising administering via intravenous infusion to the individual a composition comprising 200 mg of pembrolizumab or pembrolizumab variant and 200 mg of anti-LAG3 antibody Ab6 or Ab6 variant. In another aspect, the invention provides a method for treating cancer in a patient comprising administering via intravenous infusion to the individual a composition comprising 200 mg of pembrolizumab or pembrolizumab variant and 800 mg of anti-LAG3 antibody Ab6 or Ab6 variant.

In one embodiment, the composition comprises 200 mg of pembrolizumab or pembrolizumab variant and 200-800 mg of anti-LAG3 antibody Ab6 or Ab6 variant. In one embodiment, the composition comprises 200 mg of pembrolizumab or pembrolizumab variant and 200-700 mg of anti-LAG3 antibody Ab6 or Ab6 variant. In one embodiment, the composition comprises 200 mg of pembrolizumab or pembrolizumab variant and 100-800 mg of anti-LAG3 antibody Ab6 or Ab6 variant. In one embodiment, the composition comprises 200 mg of pembrolizumab or pembrolizumab variant and 200-900 mg of anti-LAG3 antibody Ab6 or Ab6 variant.

In one embodiment, the composition comprises 200 mg of pembrolizumab or pembrolizumab variant and 200-600 mg of anti-LAG3 antibody Ab6 or Ab6 variant. In one embodiment, the composition comprises 200 mg of pembrolizumab or pembrolizumab variant and 200-1000 mg of anti-LAG3 antibody Ab6 or Ab6 variant.

In another embodiment, the invention provides a medicament comprising the anti-LAG3 antibody for use in combination with an anti-PD-1 or anti-PD-L1 antibody for treating cancer, wherein the anti-LAG3 antibody is administered at 7-1200 mg via intravenous infusion. In another embodiment, the invention provides a medicament comprising the anti-LAG3 antibody and an anti-PD-1 antibody for treating cancer. In one embodiment, the medicament comprises 200 mg of pembrolizumab or pembrolizumab variant and 200 mg of anti-LAG3 antibody Ab6 or Ab6 variant. In another embodiment, the medicament comprises 200 mg of pembrolizumab or pembrolizumab variant and 800 mg of Ab6 or Ab6 variant. In another embodiment, the medicament comprises 400 mg of pembrolizumab or pembrolizumab variant and 800 mg of Ab6 or Ab6 variant.

In a still further embodiment, the invention provides use of the anti-LAG3 antibody and an anti-PD-1 or anti-PD-L1 antibody in the manufacture of a medicament for treating cancer in an individual. In one embodiment, the medicament comprises 200 mg of pembrolizumab or pembrolizumab variant and 200 mg of anti-LAG3 antibody Ab6 or Ab6 variant. In another aspect, the medicament comprises 200 mg of pembrolizumab or pembrolizumab variant and 800 mg of Ab6 or Ab6 variant. In another embodiment, the invention provides use of the anti-LAG3 antibody in the manufacture of a medicament for treating cancer in an individual, wherein the anti-LAG3 antibody is co-administered at 7-1200 mg via intravenous infusion with the anti-PD-1 antibody at 200 mg via intravenous infusion. In a still another embodiment, the invention provides use of the anti-LAG3 antibody in the manufacture of a medicament for treating cancer in an individual, wherein the anti-LAG3 antibody is co-administered at 200 mg via intravenous infusion with the anti-PD-1 antibody at 200 mg via intravenous infusion. In a still another embodiment, the invention provides use of the anti-LAG3 antibody in the manufacture of a medicament for treating cancer in an individual, wherein the anti-LAG3 antibody is co-administered at 800 mg via intravenous infusion with the anti-PD-1 antibody at 200 mg via intravenous infusion. In yet a further embodiment, the invention provides use of the anti-LAG3 antibody in the manufacture of a medicament for treating cancer in an individual, wherein the anti-LAG3 antibody is co-administered at 7-1200 mg via intravenous infusion with the anti-PD-1 antibody at 400 mg via intravenous infusion. In yet a further embodiment, the invention provides use of the anti-LAG3 antibody in the manufacture of a medicament for treating cancer in an individual, wherein the anti-LAG3 antibody is co-administered at 800 mg via intravenous infusion with the anti-PD-1 antibody at 400 mg via intravenous infusion. In yet a further embodiment, the invention provides use of the anti-LAG3 antibody in the manufacture of a medicament for treating cancer in an individual, wherein the anti-LAG3 antibody is co-administered at 200 mg via intravenous infusion with the anti-PD-1 antibody at 400 mg via intravenous infusion.

In the foregoing methods, medicaments and uses, in one embodiment, the anti-PD-1 antibody and anti-LAG3 antibody are co-formulated. In one embodiment, a co-formulated product with 200 mg pembrolizumab or pembrolizumab variant and 200 mg Ab6 or Ab6 variant is used for intravenous infusion. In one embodiment, a co-formulated product with 200 mg pembrolizumab or pembrolizumab variant and 300 mg Ab6 or Ab6 variant is used for intravenous infusion. In one embodiment, a co-formulated product with 200 mg pembrolizumab or pembrolizumab variant and 400 mg Ab6 or Ab6 variant is used for intravenous infusion. In another embodiment, a co-formulated product with 200 mg pembrolizumab or pembrolizumab variant and 500 mg Ab6 or Ab6 variant is used for intravenous infusion. In another embodiment, a co-formulated product with 200 mg pembrolizumab or pembrolizumab variant and 600 mg Ab6 or Ab6 variant is used for intravenous infusion. In another embodiment, a co-formulated product with 200 mg pembrolizumab or pembrolizumab variant and 700 mg Ab6 or Ab6 variant is used for intravenous infusion. In a further embodiment, a co-formulated product with 200 mg pembrolizumab or pembrolizumab variant and 800 mg Ab6 or Ab6 variant is used for intravenous infusion. In a further embodiment, a co-formulated product with 200 mg pembrolizumab or pembrolizumab variant and 900 mg Ab6 or Ab6 variant is used for intravenous infusion. In yet a further embodiment, a co-formulated product with 200 mg pembrolizumab or pembrolizumab variant and 1000 mg Ab6 or Ab6 variant is used for intravenous infusion. In yet a further embodiment, a co-formulated product with 200 mg pembrolizumab or pembrolizumab variant and 1100 mg Ab6 or Ab6 variant is used for intravenous infusion. In yet a further embodiment, a co-formulated product with 200 mg pembrolizumab or pembrolizumab variant and 1200 mg Ab6 or Ab6 variant is used for intravenous infusion.

The invention also provides a pharmaceutical composition comprising 200 mg pembrolizumab or pembrolizumab variant, and 200 mg of Ab6 or Ab6 variant, and pharmaceutically acceptable excipients. In one embodiment, the pharmaceutical composition comprises 200 mg pembrolizumab or pembrolizumab variant, and 300 mg of Ab6 or Ab6 variant, and pharmaceutically acceptable excipients. In one embodiment, the pharmaceutical composition comprises 200 mg pembrolizumab or pembrolizumab variant, and 400 mg of Ab6 or Ab6 variant, and pharmaceutically acceptable excipients. In another embodiment, the pharmaceutical composition comprises 200 mg pembrolizumab or pembrolizumab variant, and 500 mg of Ab6 or Ab6 variant, and pharmaceutically acceptable excipients. In a further embodiment, the pharmaceutical composition comprises 200 mg pembrolizumab or pembrolizumab variant, and 600 mg of Ab6 or Ab6 variant, and pharmaceutically acceptable excipients. In a further embodiment, the pharmaceutical composition comprises 200 mg pembrolizumab or pembrolizumab variant, and 700 mg of Ab6 or Ab6 variant, and pharmaceutically acceptable excipients. In a further embodiment, the pharmaceutical composition comprises 200 mg pembrolizumab or pembrolizumab variant, and 800 mg of Ab6 or Ab6 variant, and pharmaceutically acceptable excipients. In a further embodiment, the pharmaceutical composition comprises 200 mg pembrolizumab or pembrolizumab variant, and 900 mg of Ab6 or Ab6 variant, and pharmaceutically acceptable excipients. In yet a further embodiment, the pharmaceutical composition comprises 200 mg pembrolizumab or pembrolizumab variant, and 1000 mg of Ab6 or Ab6 variant, and pharmaceutically acceptable excipients. In yet a further embodiment, the pharmaceutical composition comprises 200 mg pembrolizumab or pembrolizumab variant, and 1100 mg of Ab6 or Ab6 variant, and pharmaceutically acceptable excipients. In yet a further embodiment, the pharmaceutical composition comprises 200 mg pembrolizumab or pembrolizumab variant, and 1200 mg of Ab6 or Ab6 variant, and pharmaceutically acceptable excipients.

In the foregoing methods, medicaments and uses, in another embodiment, the anti-PD-1 or anti-PD-L1 antibody and anti-LAG3 antibody are co-administered. In one embodiment, 200 mg pembrolizumab or pembrolizumab variant and 200 mg Ab6 or Ab6 variant are co-administered on Day 1 every three weeks for intravenous infusion. In one embodiment, 200 mg pembrolizumab or pembrolizumab variant and 300 mg Ab6 or Ab6 variant are co-administered on Day 1 every three weeks for intravenous infusion. In one embodiment, 200 mg pembrolizumab or pembrolizumab variant and 400 mg Ab6 or Ab6 variant are co-administered on Day 1 every three weeks for intravenous infusion. In another embodiment, 200 mg pembrolizumab or pembrolizumab variant and 500 mg Ab6 or Ab6 variant are co-administered on Day 1 every three weeks for intravenous infusion. In another embodiment, 200 mg pembrolizumab or pembrolizumab variant and 600 mg Ab6 or Ab6 variant are co-administered on Day 1 every three weeks for intravenous infusion. In a further embodiment, 200 mg pembrolizumab or pembrolizumab variant and 700 mg Ab6 or Ab6 variant are co-administered on Day 1 every three weeks for intravenous infusion. In a further embodiment, 200 mg pembrolizumab or pembrolizumab variant and 800 mg Ab6 or Ab6 variant are co-administered on Day 1 every three weeks for intravenous infusion. In a further embodiment, 200 mg pembrolizumab or pembrolizumab variant and 900 mg Ab6 or Ab6 variant are co-administered on Day 1 every three weeks for intravenous infusion. In a further embodiment, 200 mg pembrolizumab or pembrolizumab variant and 1000 mg Ab6 or Ab6 variant are co-administered on Day 1 every three weeks for intravenous infusion. In yet a further embodiment, 200 mg pembrolizumab or pembrolizumab variant and 1100 mg Ab6 or Ab6 variant are co-administered on Day 1 every three weeks for intravenous infusion. In yet a further embodiment, 200 mg pembrolizumab or pembrolizumab variant and 1200 mg Ab6 or Ab6 variant are co-administered on Day 1 every three weeks for intravenous infusion.

In the foregoing methods, medicaments and uses, in one embodiment, 400 mg pembrolizumab or pembrolizumab variant is administered on Day 1 every six weeks and 200 mg Ab6 or Ab6 variant is administered on Day 1 every three weeks for intravenous infusion. In one embodiment, 400 mg pembrolizumab or pembrolizumab variant is administered on Day 1 every six weeks and 300 mg Ab6 or Ab6 variant is administered on Day 1 every three weeks for intravenous infusion. In one embodiment, 400 mg pembrolizumab or pembrolizumab variant is administered on Day 1 every six weeks and 400 mg Ab6 or Ab6 variant is administered on Day 1 every three weeks for intravenous infusion. In another embodiment, 400 mg pembrolizumab or pembrolizumab variant is administered on Day 1 every six weeks and 500 mg Ab6 or Ab6 variant is administered on Day 1 every three weeks for intravenous infusion. In another embodiment, 400 mg pembrolizumab or pembrolizumab variant is administered on Day 1 every six weeks and 600 mg Ab6 or Ab6 variant is administered on Day 1 every three weeks for intravenous infusion. In another embodiment, 400 mg pembrolizumab or pembrolizumab variant is administered on Day 1 every six weeks and 700 mg Ab6 or Ab6 variant is administered on Day 1 every three weeks for intravenous infusion. In a further embodiment, 400 mg pembrolizumab or pembrolizumab variant is administered on Day 1 every six weeks and 800 mg Ab6 or Ab6 variant is administered on Day 1 every three weeks for intravenous infusion. In a further embodiment, 400 mg pembrolizumab or pembrolizumab variant is administered on Day 1 every six weeks and 900 mg Ab6 or Ab6 variant is administered on Day 1 every three weeks for intravenous infusion. In a further embodiment, 400 mg pembrolizumab or pembrolizumab variant is administered on Day 1 every six weeks and 1000 mg Ab6 or Ab6 variant is administered on Day 1 every three weeks for intravenous infusion. In a further embodiment, 400 mg pembrolizumab or pembrolizumab variant is administered on Day 1 every six weeks and 1200 mg Ab6 or Ab6 variant is administered on Day 1 every three weeks for intravenous infusion.

In the foregoing methods, medicaments and uses, in one embodiment, the cancer is colorectal cancer. The treatment may further comprise administration of mFOLFOX7 (Leucovorin (Calcium Folinate), Fluorouracil, Oxaliplatin) or FOLFIRI (Leucovorin (Calcium Folinate), Fluorouracil, Irinotecan Hydrochloride) in the treatment of colorectal cancer. In one embodiment, the colorectal cancer is non-microsatellite instability-high (non-MSI-H) or proficient mismatch repair (pMMR) colorectal cancer.

In one aspect, mFOLFOX7 is administered intravenously: oxaliplatin is administered at 65 or 85 mg/m², leucovorin (calcium folinate) is administered at 400 mg/m², fluorouracil (5 FU) is administered at 2000 or 2400 mg/m² every 2 weeks (Q2W). In one embodiment, leucovorin can be substituted with levofolinate calcium administered at 200 mg/m². In one embodiment, pembrolizumab or pembrolizumab variant is administered at 200 mg intravenously on Day 1 of each 21 day cycle, Ab6 or Ab6 variant is administered at 200 mg intravenously on Day 1 of each 21 day cycle, mFOLFOX7 is administered intravenously: oxaliplatin is administered at 65 or 85 mg/m², leucovorin (calcium folinate) is administered at 400 mg/m², fluorouracil (5 FU) is administered at 2000 or 2400 mg/m² on Day 1 or Day 8 every two weeks. In one embodiment, pembrolizumab or pembrolizumab variant is administered at 200 mg intravenously on Day 1 of each 21 day cycle, Ab6 or Ab6 variant is administered at 700 mg intravenously on Day 1 of each 21 day cycle, mFOLFOX7 is administered intravenously: oxaliplatin is administered at 65 or 85 mg/m², leucovorin (calcium folinate) is administered at 400 mg/m², fluorouracil (5 FU) is administered at 2000 or 2400 mg/m² on Day 1 or Day 8 every two weeks. In another embodiment, a pharmaceutical composition comprising 200 mg pembrolizumab or pembrolizumab variant and 200 mg Ab6 or Ab6 variant is administered intravenously on Day 1 of each 21 day cycle, mFOLFOX7 is administered intravenously: oxaliplatin is administered at 65 or 85 mg/m², leucovorin (calcium folinate) is administered at 400 mg/m², fluorouracil (5 FU) is administered at 2000 or 2400 mg/m² on Day 1 or Day 8 every two weeks. In another embodiment, a pharmaceutical composition comprising 200 mg pembrolizumab or pembrolizumab variant and 800 mg Ab6 or Ab6 variant is administered intravenously on Day 1 of each 21 day cycle, mFOLFOX7 is administered intravenously: oxaliplatin is administered at 65 or 85 mg/m², leucovorin (calcium folinate) is administered at 400 mg/m², fluorouracil (5 FU) is administered at 2000 or 2400 mg/m² on Day 1 or Day 8 every two weeks.

In one embodiment, pembrolizumab or pembrolizumab variant is administered at 400 mg intravenously on Day 1 every six weeks, Ab6 or Ab6 variant is administered at 200 mg intravenously on Day 1 of each 21 day cycle, mFOLFOX7 is administered intravenously: oxaliplatin is administered at 65 or 85 mg/m², leucovorin (calcium folinate) is administered at 400 mg/m², fluorouracil (5 FU) is administered at 2000 or 2400 mg/m² on Day 1 or Day 8 every two weeks. In one embodiment, pembrolizumab or pembrolizumab variant is administered at 400 mg intravenously on Day 1 every six weeks, Ab6 or Ab6 variant is administered at 700 mg intravenously on Day 1 of each 21 day cycle, mFOLFOX7 is administered intravenously: oxaliplatin is administered at 65 or 85 mg/m², leucovorin (calcium folinate) is administered at 400 mg/m², fluorouracil (5 FU) is administered at 2000 or 2400 mg/m² on Day 1 or Day 8 every two weeks.

In another aspect, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² every 2 weeks (Q2W). In one embodiment, pembrolizumab or pembrolizumab variant is administered at 200 mg intravenously on Day 1 of each 21 day cycle, Ab6 or Ab6 variant is administered at 200 mg intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks. In one embodiment, pembrolizumab or pembrolizumab variant is administered at 200 mg intravenously on Day 1 of each 21 day cycle, Ab6 or Ab6 variant is administered at 700 mg intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks. In another embodiment, pembrolizumab or pembrolizumab variant is administered at 200 mg intravenously on Day 1 of each 21 day cycle, Ab6 or Ab6 variant is administered at 800 mg intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks. In another embodiment, a pharmaceutical composition comprising 200 mg pembrolizumab or pembrolizumab variant and 200 mg Ab6 or Ab6 variant is administered intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks. In another embodiment, a pharmaceutical composition comprising 200 mg pembrolizumab or pembrolizumab variant and 800 mg Ab6 or Ab6 variant is administered intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks.

In one embodiment, pembrolizumab or pembrolizumab variant is administered at 400 mg intravenously on Day 1 every six weeks, Ab6 or Ab6 variant is administered at 200 mg intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks. In one embodiment, pembrolizumab or pembrolizumab variant is administered at 400 mg intravenously on Day 1 every six weeks, Ab6 or Ab6 variant is administered at 700 mg intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks. In another embodiment, pembrolizumab or pembrolizumab variant is administered at 400 mg intravenously on Day 1 every six weeks, Ab6 or Ab6 variant is administered at 800 mg intravenously on Day 1 of each 21 day cycle, FOLFIRI is administered intravenously: irinotecan is administered at 150 or 180 mg/m², leucovorin (calcium folinate) is administered at 400 mg/m², fluorouracil (5 FU) is administered at 2000 or 2400 mg/m² on Day 1 or Day 8 every two weeks.

Cancers that may be treated by the antibodies, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome); hematopoietic tumors of the lymphoid lineage, include leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoetic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma, and schwannomas; and other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma. In one embodiment, the forgoing cancers are advanced, unresectable or metastatic. In one embodiment, the patients are refractory to anti-PD-1 or anti-PD-L1 therapy.

In one embodment, cancers that may be treated by the antibodies, compositions and methods of the invention include, but are not limited to: lung cancer, pancreatic cancer, colon cancer, colorectal cancer, myeloid leukemias, acute myelogenous leukemia, chronic myelogenous leukemia, chronic myelomonocytic leukemia, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

In another embodiment, cancers that may be treated by the antibodies, compositions and methods of the invention include, but are not limited to: head and neck squamous cell cancer, gastric cancer, adenocarcinoma of the stomach and/or gastric-esophageal junction, renal cell cancer, fallopian tube cancer, endometrial cancer, and colorectal cancer. In one embodiment, the colorectal cancer, gastric cancer, adenocarcinoma of the stomach and/or gastric-esophageal junction (GEJ), or endometrial cancer is non-microsatellite instability-high (non-MSI-H) or proficient mismatch repair (pMMR). In one embodiment, the cancer is gastric cancer, adenocarcinoma of the stomach and/or gastric-esophageal junction. In one embodiment, the cancer is renal cell carcinoma. In one embodiment, the patient with head and neck squamous cell cancer is anti-PD-1 or anti-PD-L1 treatment refractory. In one embodiment, the patient with head and neck squamous cell cancer has not received prior anti-PD-1 or anti-PD-L1 treatment. In one embodiment, the colorectal cancer is unresectable or metastatic (Stage IV). In one embodiment, the cancer is non-small cell lung cancer.

In another embodiment, cancers that may be treated by the antibodies, compositions and methods of the invention include hematological malignancies, but are not limited to: classical Hodgkin lymphoma (cHL), diffuse large B-cell lymphoma (DLBCL), transformed DLBCL, gray zone lymphoma, double hit lymphoma, Primary mediastinal B cell lymphoma (PMBCL) or indolent non-Hodgkin lymphoma (iNHL) (for example, follicular lymphoma, marginal zone lymphoma, mucosa-associated lymphoid tissue lymphoma, or small lymphocytic lymphoma). In one embodiment, the patient with Hodgkin lymphoma is anti-PD-1 or anti-PD-L1 treatment refractory.

In a further embodiment, cancers that may be treated by the antibodies, compositions and methods of the invention include cancers selected from the group consisting of: renal cell carcinoma, urothelial carcinoma of the renal pelvis, ureter, bladder or urethra, melanoma, gastric, GEJ adenocarcinoma, non-small cell lung cancer and bladder cancer. In a further embodiment, cancers that may be treated are selected from the group consisting of: renal cell carcinoma, gastric, GEJ adenocarcinoma, non-small cell lung cancer, head and neck squamous cell cancer, fallopian tube cancer, endometrial cancer, and colorectal cancer. In one embodiment, the colorectal cancer is non-microsatellite instability-high (non-MSI-H) or proficient mismatch repair (pMMR). In one embodiment, the forgoing cancers are advanced, unresectable or metastatic. In one embodiment, the non-small cell lung cancer is advanced or Stage IV. In another embodiment, the melanoma is advanced or Stage III. In one embodiment, the patients are refractory to anti-PD-1 or anti-PD-L1 therapy.

In one embodiment, a co-formulated product with 200 mg pembrolizumab or pembrolizumab variant and 200 mg Ab6 or Ab6 variant is used. In another embodiment, a co-formulated product with 200 mg pembrolizumab or pembrolizumab variant and 600 mg Ab6 or Ab6 variant is used. In one embodiment, a co-formulated product with 200 mg pembrolizumab or pembrolizumab variant and 700 mg Ab6 or Ab6 variant is used. In another embodiment, a co-formulated product with 200 mg pembrolizumab or pembrolizumab variant and 800 mg Ab6 or Ab6 variant is used. In another embodiment, a co-formulated product with 200 mg pembrolizumab or pembrolizumab variant and 1000 mg Ab6 or Ab6 variant is used.

In a further embodiment, the cancer is non-small cell lung cancer, and the patient lacks tumor activating epidermal growth factor receptor (EGFR), or B isoform of rapidly accelerated fibrosarcoma (B-Raf) mutations and lacks anaplastic lymphoma kinase (ALK) or c-ros oncogene 1 (ROS1) gene rearrangements. In a further embodiment, the cancer is non-small cell lung cancer, and the tumor has a squamous histology.

The combination therapy may also comprise one or more additional therapeutic agents. The additional therapeutic agent may be, e.g., a chemotherapeutic, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-C SF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the anti-LAG3 antibody is administered before administration of the anti-PD-1 antibody or anti-PD-L1 antibody, while in other embodiments, the anti-LAG3 antibody is administered after administration of the anti-PD-1 antibody or anti-PD-L1 antibody. In another embodiment, the anti-LAG3 antibody is administered concurrently with the anti-PD-1 antibody or anti-PD-L1 antibody.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration. A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naive. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as MRI, ultrasound, or CAT scan.

A combination therapy of the invention is preferably administered to a human patient who has a cancer that tests positive for one or both of PD-L1 and PD-L2, and preferably tests positive for PD-L1 expression. In some preferred embodiments, PD-L1 expression is detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the anti-PD-1 antibody or anti-PD-L1 antibody and anti-LAG3 antibody, but it is envisioned that the physician could order the first or subsequent diagnostic tests on a tunor tissue section at any time after initiation of treatment, such as for example after completion of a treatment cycle. In one embodiment, the PD-L1 expression is measured by the PD-L1 IHC 22C3 pharmDx assay. In another embodiment, the patient has a Mononuclear Inflammatory Density Score for PD-L1 expression≥2. In another embodiment, the patient has a Mononuclear Inflammatory Density Score for PD-L1 expression≥3. In another embodiment, the patient has a Mononuclear Inflammatory Density Score for PD-L1 expression≥4. In another embodiment, the patient has a Tumor Proportion Score for PD-L1 expression≥1%. In another embodiment, the patient has a Tumor Proportion Score for PD-L1 expression≥10%. In another embodiment, the patient has a Tumor Proportion Score for PD-L1 expression≥20%. In another embodiment, the patient has a Tumor Proportion Score for PD-L1 expression≥30%. In another embodiment, the patient has a Tumor Proportion Score for PD-L1 expression≥50%. In a further embodiment, the patient has a Combined Positive Score for PD-L1 expression≥1%. In another embodiment, the patient has a Mononuclear Inflammatory Density Score for PD-L1 expression≥2 or a Tumor Proportion Score for PD-L1 expression≥1%. In a further embodiment, the patient has a Combined Positive Score for PD-L1 expression between 1 and 20%. In a further embodiment, the patient has a Combined Positive Score for PD-L1 expression≥2%. In a further embodiment, the patient has a Combined Positive Score for PD-L1 expression≥5%. In yet a further embodiment, the patient has a Combined Positive Score for PD-L1 expression≥10%. In a further embodiment, the patient has a Combined Positive Score for PD-L1 expression≥15%. In yet a further embodiment, the patient has a Combined Positive Score for PD-L1 expression≥20%. In another embodiment, the patient has non-small cell lung cancer and a Tumor Proportion Score for PD-L1 expression≥50%.

In addition, the combination therapy of the invention can be administered to a human patient who has a cancer that tests positive for LAG3 expression. In some preferred embodiments, LAG3 expression is detected using a diagnostic anti-human LAG3 antibody, or antigen binding fragment thereof, in an IHC assay on an FFPE or frozen tissue section of a tumor sample removed from the patient. Typically, the patient's physician would order a diagnostic test to determine LAG3 expression in a tumor tissue sample removed from the patient prior to initiation of treatment with the anti-PD-1 antibody or anti-PD-L1 antibody and anti-LAG3 antibody, but it is envisioned that the physician could order the first or subsequent diagnostic tests at any time after initiation of treatment, such as for example after completion of a treatment cycle. In one embodiment, the patient has a CPS-like LAG3% positive cells of ≥1%. In one embodiment, the patient has a CPS-like LAG3% positive cells of ≥2%. In one embodiment, the patient has a CPS-like LAG3% positive cells of ≥5%. In one embodiment, the patient has a CPS-like LAG3% positive cells of ≥10%. In one embodiment, the patient has a LAG3% positive cells of ≥1%. In one embodiment, the patient has a LAG3% positive cells of ≥2%. In one embodiment, the patient has a LAG3% positive cells of ≥5%. In one embodiment, the patient has a LAG3% positive cells of ≥10%.

In one preferred embodiment of the invention, the anti-PD-1 antibody in the combination therapy is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg/kg Q3W.

In another preferred embodiment of the invention, the anti-PD-1 antibody in the combination therapy is pembrolizumab, or a pembrolizumab variant, which is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W and flat-dose equivalents of any of these doses, i.e., such as 200 mg Q3W. In some embodiments, pembrolizumab is provided as a liquid medicament which comprises 25 mg/ml pembrolizumab, 7% (w/v) sucrose, 0.02% (w/v) polysorbate 80 in 10 mM histidine buffer pH 5.5. In other embodiments, pembrolizumab is provided as a liquid medicament which comprises about 125 to about 200 mg/mL of pembrolizumab, or antigen binding fragment thereof; about 10 mM histidine buffer; about 10 mM L-methionine, or a pharmaceutically acceptable salt thereof; about 7% w/v sucrose; and about 0.02% w/v polysorbate 80.

In some embodiments of the invention, the anti-PD-1 antibody, or antigen binding fragment thereof is administered to the patient once every four or six weeks for 12 weeks or more. In other embodiments, the anti-PD-1 antibody, or antigen binding fragment thereof is administered to the patient once every six weeks for 16 weeks or more, 18 weeks or more, 20 weeks or more, 24 weeks or more, 28 weeks or more, 30 weeks or more, 32 weeks or more, 36 weeks or more, 40 weeks or more, 42 weeks or more, 44 weeks or more, 48 weeks or more, 52 weeks or more, 54 weeks or more, 56 weeks or more, 60 weeks or more, 64 weeks or more, 66 weeks or more, 68 weeks or more, 72 weeks or more, 76 weeks or more, 78 weeks or more, 80 weeks or more, 84 weeks or more, 88 weeks or more, or 90 weeks or more. In other embodiments, the anti-PD-1 antibody, or antigen binding fragment thereof is administered at 400 mg every six weeks.

In some embodiments, the selected dose of pembrolizumab is administered by IV infusion. In one embodiment, the selected dose of pembrolizumab is administered by IV infusion over a time period of between 25 and 40 minutes, or about 30 minutes.

In some embodiments, the patient is treated with the combination therapy for at least 24 weeks, e.g., eight 3-week cycles. In some embodiments, treatment with the combination therapy continues until the patient exhibits evidence of PD or a CR.

Pharmaceutically acceptable excipients of the present disclosure include for instance, solvents, bulking agents, buffering agents, tonicity adjusting agents, and preservatives (see, e.g.,. Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab that are suitable for use in the present invention. In some embodiments, a medicament comprising pembrolizumab is provided in a glass vial which contains about 100 mg of pembrolizumab in 4 ml of solution. Each 1 mL of solution contains 25 mg of pembrolizumab and is formulated in: L-histidine (1.55 mg), polysorbate 80 (0.2 mg), sucrose (70 mg), and Water for Injection, USP. The solution requires dilution for IV infusion.

In some embodiments, a medicament comprising the anti-LAG3 antibody may be provided as a liquid formulation or prepared by reconstituting a lyophilized powder with sterile water for injection prior to use. In one embodiment, the liquid formulation comprises about 25 mg/mL anti-LAG3 antibody; about 50 mg/mL sucrose; about 0.2 mg/mL polysorbate 80; about 10 mM L-histidine buffer at about pH 5.8-6.0; about 70 mM L-Arginine-HCl thereof; and optionally about 10 mM L-methionine.

The medicaments described herein may be provided as a kit which comprises a first container and a second container and a package insert. The first container contains at least one dose of a medicament comprising a PD-1 antagonist, the second container contains 7-1200 mg of a medicament comprising the anti-LAG3 antibody, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some preferred embodiments of the kit, the PD-1 antagonist is an anti-PD-1 antibody and the instructions state that the medicaments are intended for use in treating a patient having cancer that tests positive for PD-L1 expression by an IHC assay.

In other aspects, the medicament is a co-formulation of anti-LAG3 antibodies or antigen binding fragments and anti-PD-1 antibodies or antigen binding fragments with arginine or a pharmaceutically acceptable salt thereof at a total concentration of 10-1000 mM, and a buffer at pH about 5-8, and optionally 3-100 mM of methionine. In one embodiment, the co-formulation comprises about 10 to 120 mg/mL of an anti-LAG3 antibody; about 10 to 120 mg/mL of an anti-PD-1 antibody; about 30 to 120 mg/mL sucrose or trehalose; about 0.05 to 2 mg/mL polysorbate 80; about 3 to 30 mM L-histidine buffer at pH about 5.0-6.5; about 40 to 150 mM L-arginine or a pharmaceutically acceptable salt thereof; and optionally, about 5 to 70 mM L-methionine. WO 2018/204374 describes the preparation of liquid and lyophilized medicaments comprising Ab6, or Ab6 co-formulated with pembrolizumab that are suitable for use in the present invention.

These and other aspects of the invention, including the exemplary specific embodiments listed below, will be apparent from the teachings contained herein.

### Exemplary Specific Embodiments of the Invention

1. An anti-LAG3 antibody for use in the treatment of cancer in a patient, wherein the anti-LAG3 antibody comprises: (a) light chain CDRs of SEQ ID NOs: 26, 27 and 28 and (b) heavy chain CDRs of SEQ ID NOs: 29, 30 and 31, and is administered at 7-1200 mg via intravenous infusion.
2. The anti-LAG3 antibody for use of embodiment 1, wherein the patient is administered 100 mg of the anti-LAG3 antibody.
3. The anti-LAG3 antibody for use of embodiment 1, wherein the patient is administered 200 mg of the anti-LAG3 antibody.
4. The anti-LAG3 antibody for use of embodiment 1, wherein the patient is administered 700 mg of the anti-LAG3 antibody.
5. The anti-LAG3 antibody for use of embodiment 1, wherein the patient is administered 800 mg of the anti-LAG3 antibody.
6. The anti-LAG3 antibody for use of embodiments 1 to 5, wherein the patient is administered the anti-LAG3 antibody on Day 1 once every three weeks.
7. The anti-LAG3 antibody for use of any one of embodiments 1 to 6, wherein the anti-LAG3 antibody comprises a heavy chain and a light chain, and wherein the heavy chain comprises a heavy chain variable region comprising SEQ ID NO:25 and the light chain comprises a light chain variable region comprising SEQ ID NO: 24.
8. The anti-LAG3 antibody for use of any one of embodiments 1 to 6, wherein the anti-LAG3 antibody comprises a heavy chain and a light chain, and wherein the heavy chain comprises SEQ ID NO:23 and the light chain comprises SEQ ID NO:22.
9. The anti-LAG3 antibody for use of any one of embodiments 1 to 6, wherein the anti-LAG3 antibody is an Ab6 variant.
10. The anti-LAG3 antibody for use of any one of embodiments 1 to 9, wherein the anti-LAG3 antibody is co-administered with an anti-PD-1 antibody or anti-PD-L1 antibody, or antigen binding fragment thereof.
11. The anti-LAG3 antibody for use of embodiments 1 to 9, wherein the anti-LAG3 antibody is co-formulated with an anti-PD-1 antibody or anti-PD-L1 antibody or antigen binding fragment thereof.
12. The anti-LAG3 antibody for use of embodiment 10 or 11, wherein the anti-PD-1 antibody, or antigen binding fragment thereof specifically binds to human PD-1 and blocks the binding of human PD-L1 to human PD-1.
13. The anti-LAG3 antibody for use of embodiment 12, wherein the anti-PD-1 antibody, or antigen binding fragment thereof also blocks binding of human PD-L2 to human PD-1.
14. The anti-LAG3 antibody for use of embodiment 13, wherein the anti-PD-1 antibody, or antigen binding fragment thereof comprises: (a) light chain CDRs of SEQ ID NOs: 1, 2 and 3 and (b) heavy chain CDRs of SEQ ID NOs: 6, 7 and 8.
15. The anti-LAG3 antibody for use of embodiment 13, wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4.
16. The anti-LAG3 antibody for use of embodiment 13, wherein the anti-PD-1 antibody comprises a heavy chain and a light chain, and wherein the heavy chain comprises SEQ ID NO:10 and the light chain comprises SEQ ID NO:5.
17. The anti-LAG3 antibody for use of embodiment 13, wherein the anti-PD-1 antibody is pembrolizumab.
18. The anti-LAG3 antibody for use of embodiment 13, wherein the anti-PD-1 antibody is a pembrolizumab variant.
19. The anti-LAG3 antibody for use of embodiment 10, wherein the anti-PD-1 antibody is nivolumab.
20. The anti-LAG3 antibody for use of embodiment 10, wherein the anti-PD-L1 antibody is atezolizumab, durvalumab, or avelumab.
21. The anti-LAG3 antibody for use of any one of embodiments 14-18, wherein the anti-PD-1 antibody is administered at 200 mg via intravenous infusion on Day 1 once every three weeks.
22. The anti-LAG3 antibody for use of any one of embodiments 14-18, wherein the anti-PD-1 antibody is administered at 400 mg via intravenous infusion on Day 1 once every six weeks.
23. The anti-LAG3 antibody for use of embodiment 10 or 11, wherein the anti-PD-1 antibody is a humanized anti-PD-1 antibody that comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1, 2 and 3; and the anti-LAG3 antibody is a humanized anti-LAG3 antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.
24. The anti-LAG3 antibody for use of embodiment 10 or 11, wherein the anti-PD-1 antibody comprises a heavy chain and a light chain, and wherein the heavy chain comprises a heavy chain variable region comprising SEQ ID NO :9 and the light chain comprises a light chain variable region comprising SEQ ID NO: 4; and the anti-LAG3 antibody comprises a heavy chain and a light chain, and wherein the heavy chain comprises a heavy chain variable region comprising SEQ ID NO:25 and the light chain comprises a light chain variable region comprising SEQ ID NO: 24.
25. The anti-LAG3 antibody for use of embodiment 10 or 11, wherein the anti-PD-1 antibody comprises a heavy chain and a light chain, and wherein the heavy chain comprises SEQ ID NO:10 and the light chain comprises SEQ ID NO: 5; and the anti-LAG3 antibody comprises a heavy chain and a light chain, and wherein the heavy chain comprises SEQ ID NO:23 and the light chain comprises SEQ ID NO: 22.
26. The anti-LAG3 antibody for use of any one of embodiments 23-25, wherein the anti-PD-1 antibody is administered at 200 mg via intravenous infusion on Day 1 once every three weeks, and the anti-LAG3 antibody is administered at 200 mg via intravenous infusion on Day 1 once every three weeks.
27. The anti-LAG3 antibody for use of any one of embodiments 23-25, wherein the anti-PD-1 antibody is administered at 400 mg via intravenous infusion on Day 1 once every six weeks, and the anti-LAG3 antibody is administered at 200 mg via intravenous infusion on Day 1 once every three weeks.
28. The anti-LAG3 antibody for use of of any one of embodiments 23-25, wherein the anti-PD-1 antibody is administered at 200 mg via intravenous infusion on Day 1 once every three weeks, and the anti-LAG3 antibody is administered at 700 or 800 mg via intravenous infusion on Day 1 once every three weeks.
29. The anti-LAG3 antibody for use of any one of embodiments 23-25, wherein the anti-PD-1 antibody is administered at 400 mg via intravenous infusion on Day 1 once every six weeks, and the anti-LAG3 antibody is administered at 700 or 800 mg via intravenous infusion on Day 1 once every three weeks.
30. The anti-LAG3 antibody for use of any one of embodiments 23-25, wherein 200 mg of anti-PD-1 antibody is co-formulated with 200 mg anti-LAG3 antibody.
31. The anti-LAG3 antibody for use of any one of embodiments 23-25, wherein 200 mg of anti-PD-1 antibody is co-formulated with 800 mg anti-LAG3 antibody.
32. The anti-LAG3 antibody for use of any one of embodiments 1 to 31, wherein the cancer is selected from the group consisting of: head and neck squamous cell cancer, gastric cancer, adenocarcinoma of the stomach and/or gastric-esophageal junction, renal cell cancer, fallopian tube cancer, endometrial cancer, and non-microsatellite instability-high (non-MSI-H) or proficient mismatch repair (pMMR) colorectal cancer.
33. The anti-LAG3 antibody for use of any one of embodiments 1 to 31, wherein the cancer is selected from the group consisting of: renal cell carcinoma, urothelial carcinoma of the renal pelvis, ureter, bladder or urethra, melanoma, gastric, non-small cell lung cancer and bladder cancer.
34. The anti-LAG3 antibody for use of any one of embodiments 1 to 31, wherein the cancer is classical Hodgkin lymphoma (cHL), diffuse large B-cell lymphoma (DLBCL), or indolent non-Hodgkin lymphoma (iNHL).
35. The anti-LAG3 antibody for use of any one of embodiments 1 to 34, wherein the individual has not been previously treated with anti-PD-1 or anti-PD-L1 therapy or is confirmed progressive while receiving prior anti-PD-1 or anti-PD-L1 therapy.
36. The anti-LAG3 antibody for use of any one of embodiments 1 to 35, wherein the tumor cells of the individual is PD-L1 expression positive.
37. The anti-LAG3 antibody for use of any one of embodiments 1 to 36, wherein the individual has a Mononuclear Inflammatory Density Score for PD-L1 expression ≥2.
38. The anti-LAG3 antibody for use of any one of embodiments 1 to 37, wherein the individual has a Combined Positive Score for PD-L1 expression ≥1%.
39. The anti-LAG3 antibody for use of any one of embodiments 1 to 37, wherein the individual has a Combined Positive Score for PD-L1 expression ≥10%.
40. The anti-LAG3 antibody for use of any one of embodiments 37-39, wherein the PD-L1 expression is measured by the PD-L1 IHC 22C3 pharmDx assay.
41. A pharmaceutical composition comprising 200 mg pembrolizumab or pembrolizumab variant, and 200 mg of Ab6 or Ab6 variant, and a pharmaceutically acceptable excipient.
42. A pharmaceutical composition comprising 200 mg pembrolizumab or pembrolizumab variant, and 800 mg of Ab6 or Ab6 variant, and a pharmaceutically acceptable excipient.
43. An anti-LAG3 antibody for use in combination with an anti-PD-1 antibody for the treatment of gastric cancer in a patient, wherein the tumor tissue section of the patient is PD-L1 expression positive.
44. The anti-LAG3 antibody for use of embodiment 43, wherein the gastric cancer is adenocarcinoma of the stomach and/or gastric-esophageal junction adenocarcinoma
45. An anti-LAG3 antibody for use in combination with an anti-PD-1 antibody for the treatment of head and neck squamous cell carcinoma in a patient, wherein the tumor tissue section of the patient is PD-L1 expression positive.
46. An anti-LAG3 antibody for use in combination with an anti-PD-1 antibody for the treatment of non-microsatellite instability-high (non-MSI-H) or proficient mismatch repair (pM_MR) colorectal cancer in a patient, wherein the tumor tissue section of the patient is PD-L1 expression positive, and the % LAG3 positive cells or CPS-like % LAG3 positive cells is ≥1%.
47. The anti-LAG3 antibody for use of embodiments 43-46, wherein the patient has not previously received therapy with an anti-PD-1 antibody or an anti-PD-L1 antibody.
48. The anti-LAG3 antibody for use of embodiments 43-47, wherein the tumor tissue section of the patient has a Combined Positive Score (CPS) for PD-L1 expression ≥1%.
49. The anti-LAG3 antibody for use of embodiments 43-47, wherein the tumor tissue section of the patient has a Combined Positive Score for PD-L1 expression ≥5%.
50. The anti-LAG3 antibody for use of embodiments 43-47, wherein the tumor tissue section of the patient has a Combined Positive Score for PD-L1 expression ≥10%.
51. The anti-LAG3 antibody for use of embodiments 43-47, wherein the tumor tissue section of the patient has a Combined Positive Score for PD-L1 expression ≥20%.
52. The anti-LAG3 antibody for use of embodiments 43-47, wherein the tumor tissue section of the patient has a Tumor Proportion Score (TPS) ≥1% or a Mononuclear Inflammatory Density Score (MIDS) ≥2%.
53. The anti-LAG3 antibody for use of embodiments 43-52, wherein the PD-L1 expression is measured by the PD-L1 IHC 22C3 pharmDx assay.
54. The anti-LAG3 antibody for use of embodiments 43-53, wherein the % LAG3 positive cells of the tumor tissue section is ≥1%.
55. The anti-LAG3 antibody for use of embodiments 43-53, wherein the CPS-like % LAG3 positive cells of the tumor tissue section is ≥1%.
56. The anti-LAG3 antibody for use of embodiments 43-55, wherein the anti-PD-1 antibody, or antigen binding fragment thereof specifically binds to human PD-1 and blocks the binding of human PD-L1 to human PD-1.
57. The anti-LAG3 antibody for use of embodiment 56, wherein the anti-PD-1 antibody, or antigen binding fragment thereof also blocks binding of human PD-L2 to human PD-1.
58. The anti-LAG3 antibody for use of embodiment 57, wherein the anti-PD-1 antibody, or antigen binding fragment thereof comprises: (a) light chain CDRs of SEQ ID NOs: 1, 2 and 3 and (b) heavy chain CDRs of SEQ ID NOs: 6, 7 and 8.
59. The anti-LAG3 antibody for use of embodiment 57, wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4.
60. The anti-LAG3 antibody for use of embodiment 57, wherein the anti-PD-1 antibody comprises a heavy chain and a light chain, and wherein the heavy chain comprises SEQ ID NO:10 and the light chain comprises SEQ ID NO:5.
61. The anti-LAG3 antibody for use of embodiment 57, wherein the anti-PD-1 antibody is pembrolizumab.
62. The anti-LAG3 antibody for use of embodiment 57, wherein the anti-PD-1 antibody is a pembrolizumab variant.
63. The anti-LAG3 antibody for use of embodiment 57, wherein the anti-PD-1 antibody is nivolumab.
64. The anti-LAG3 antibody for use of any one of embodiments 43-63, wherein the anti-LAG3 antibody comprises a heavy chain and a light chain, and wherein the heavy chain comprises a heavy chain variable region comprising SEQ ID NO:25 and the light chain comprises a light chain variable region comprising SEQ ID NO: 24.
65. The anti-LAG3 antibody for use of any one of embodiments 43-63, wherein the anti-LAG3 antibody comprises a heavy chain and a light chain, and wherein the heavy chain comprises SEQ ID NO:23 and the light chain comprises SEQ ID NO:22.
66. The anti-LAG3 antibody for use of any one of embodiments 43-63, wherein the anti-LAG3 antibody is an Ab6 variant.
67. The anti-LAG3 antibody for use of any one of embodiments 43-66, wherein the anti-LAG3 antibody is co-administered with an anti-PD-1 antibody, or antigen binding fragment thereof.
68. The anti-LAG3 antibody for use of any one of embodiments 43-66, wherein the anti-LAG3 antibody is co-formulated with an anti-PD-1 antibody or antigen binding fragment thereof.

### General Methods

Standard methods in molecular biology are described Sambrook, Fritsch and Maniatis (1982 & 1989 2nd Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed 68:177-181; von Heijne (1983) Eur. J. Biochem. 133:17-21; von Heijne (1986) Nucleic Acids Res. 14:4683-4690).

### EXAMPLES

### Example 1

### Clinical Studies of Anti-LAG3 Antibody in Advanced Solid Tumors

This is a multisite, open-label, dose-escalation study of anti-LAG3 antibody Ab6 monotherapy (Part A, Arm 1) and Ab6 in combination with pembrolizumab (Part A, Arm 2) followed by both nonrandomized and randomized dose confirmation of Ab6 in combination with pembrolizumab along with efficacy evaluations of Ab6 as monotherapy and in combination with pembrolizumab (Part B) in subjects with a histologically or cytologically confirmed diagnosis of advanced solid tumors.

During Part A of the study, subjects were allocated by nonrandom assignment to 1 of 2 treatment arms:
Arm 1: Ab6 as monotherapy escalating doses 7, 21, 70, 210 or 700 mg every 3 weeks (Q3W) via intravenous infusion (IV).
Arm 2: Ab6 escalating doses 7, 21, 70, 210 or 700 mg every 3 weeks (Q3W) IV in combination with pembrolizumab (200 mg Q3W) IV
   Part B was a dose confirmation of Ab6 in combination with pembrolizumab. Additionally, expansion cohorts assesses the antitumor efficacy of Ab6 as monotherapy and in combination with pembrolizumab. Part B consists of 5 treatment arms:

TABLE 4
Trial Treatments

| | | | | | |
|---|---|---|---|---|---|
| | | | | Regimen/ | |
| | Dose/ | Dose | Route of | Treatment | |
| Drug | Potency | Frequency | Administration | Period | Use |

Part A, Arm 1

| | | | | | |
|---|---|---|---|---|---|
| Ab6 | 7 mg | Q3W | Intravenous (IV) | Day 1 of each | Experimental |
| | 21 mg | | Infusion | 21-day cycle | |
| | 70 mg | | | | |
| | 210 mg | | | | |
| | 700 mg | | | | |

Part A, Arm 2

| | | | | | |
|---|---|---|---|---|---|
| Ab6 | 7 mg | Q3W | Intravenous (IV) | Day 1 of each | Experimental |
| | 21 mg | | Infusion | 21-day cycle | |
| | 70 mg | | | | |
| | 210 mg | | | | |
| | 700 mg | | | | |
| Pembrolizumab | 200 mg | Q3W | IV Infusion | Day 1 of each | Experimental |
| | | | | 21-day cycle | |

Part B, Arm 1

| | | | | | |
|---|---|---|---|---|---|
| Ab6 | 800 mg | Q3W | IV infusion | Day 1 of each | Experimental |
| | | | | 21-day cycle | |

Part B, Arm 2A

| | | | | | |
|---|---|---|---|---|---|
| Ab6 | 200 mg | Q3W | IV infusion | Day 1 of each | Experimental |
| | | | | 21-day cycle | |
| Pembrolizumab | 200 mg | Q3W | IV Infusion | Day 1 of each | Experimental |
| | | | | 21-day cycle | |

Part B, Arm 2B

| | | | | | |
|---|---|---|---|---|---|
| Ab6 | 700 mg | Q3W | IV infusion | Day 1 of each | Experimental |
| | | | | 21-day cycle | |
| Pembrolizumab | 200 mg | Q3W | IV Infusion | Day 1 of each | Experimental |
| | | | | 21-day cycle | |

Part B, Arm 2C

| | | | | | |
|---|---|---|---|---|---|
| Ab6 | 800 mg | Q3W | IV infusion | Day 1 of each | Experimental |
| | | | | 21-day cycle | |
| Pembrolizumab | 200 mg | Q3W | IV Infusion | Day 1 of each | Experimental |
| | | | | 21-day cycle | |

Part B, Arm 3

| Ab6 | | 800 mg | Q3W | IV infusion | Day 1 of each | Experimental |
|---|---|---|---|---|---|---|
| | | | | | 21-day cycle | |
| Pembrolizumab | | 200 mg | Q3W | IV Infusion | Day 1 of each | Experimental |
| | | | | | 21-day cycle | |
| mFOLFOX 7 | Oxaliplatin | 85 mg/m² | Q2W | IV Infusion | Odd Number | Background |
| | | 65 mg/m² | | | Cycles: Day | Therapy |
| | | | | | 1, Day 15 | |
| | | | | | Even Number | |
| | | | | | Cycles: Day 8 | |
| | Leucovorin | 400 mg/m² | | IV Infusion | | |
| | ^{c}(Calcium | | | | | |
| | Folinate) | | | | | |
| | 5-FU | 2400 mg/m² | | IV Infusion | | |
| | | 2000 mg/m² | | | | |

Part B, Arm 4

| Ab6 | | 800 mg | Q3W | IV infusion | Day 1 of each | Experimental |
|---|---|---|---|---|---|---|
| | | | | | 21-day cycle | |
| Pembrolizumab | | 200 mg | Q3W | IV Infusion | Day 1 of each | Experimental |
| | | | | | 21-day cycle | |
| FOLFIRI | Irinotecan | 180 mg/m² | Q2W | IV Infusion | Odd Number | Background |
| | | 150 mg/m² | | | Cycles: Day | Therapy |
| | | | | | 1, Day 15 | |
| | | | | | Even Number | |
| | | | | | Cycles: Day 8 | |
| | Leucovorin | 400 mg/m² | | IV Infusion | | |
| | ^{c}(Calcium | | | | | |
| | Folinate) | | | | | |
| | 5-FU | 2400 mg/m² | | IV Infusion | | |
| | | 2000 mg/m² | | | | |

Part B, Arm 5

| | | | | | |
|---|---|---|---|---|---|
| Ab6A | 800 mg | Q3W | IV infusion | Day 1 of each | Experimental |
| | Ab6 + | | | 21-day cycle | |
| | 200 mg | | | | |
| | pembro- | | | | |
| | lizumab | | | | |

^{c}Depending on local practice guidelines, levofolinate calcium (200 mg/m² Q2W) may be substituted for leucovorin.

This trial used an adaptive design based on the pre-specified criteria of dose limiting toxicity (DLT). For dose escalation (Part A, Arm 1 and Arm 2), a 3+3 dose escalation design was utilized. For dose confirmation (Part B), the toxicity probability interval (TPI) design is utilized to refine the estimate of a preliminary recommended Phase 2 dose (RPTD) from Part A, Arm 2. Additionally, Part B compares the safety and antitumor efficacy of 2 doses of Ab6 in combination with pembrolizumab.

In Part A, Arm 1 (Ab6 monotherapy), the study began with a 3+3 design to identify a preliminary maximum tolerated dose (MTD) or maximum administered dose (MAD). During 3+3 dose escalation in both arms of Part A, an initial cohort of 3 subjects were enrolled to a dose level. If none of the 3 subjects experienced a DLT during the first 21 day cycle, escalation to the next dose occurred. If 1 of the 3 subjects experienced a DLT, another 3 subjects enrolled at this dose level. If 1 DLT was observed among the 6 subjects, the dose escalation continued. If more than 1 of 3 or more than 1 of 6 subjects at a dose level developed DLTs, dose escalation was terminated, and the study proceeded at the previous dose level.

Treatment in Part A, Arm 2 (Ab6 in combination with pembrolizumab) began with a 3+3 design to identify a preliminary RPTD for Part B. The starting dose of Ab6 was at least 1 dose level below that being tested in Part A, Arm 1. A fixed dose of 200 mg pembrolizumab was used in Part A, Arm 2.

Doses of Ab6 in combination with pembrolizumab was at least 1 dose level behind the monotherapy dose, and would not exceed the MTD or MAD of Part A, Arm 1. However, once the MTD or MAD for Part A, Arm 1 was established, the dose of Ab6 in Part A, Arm 2 continued escalation up to that dose. For enrollment to the last 2 dose levels of Arm 2, all 3 (or 6) subjects in the second highest dose level completed 1 cycle of treatment and DLT evaluation before the highest dose level began enrollment.

In Part B, dose confirmation and preliminary antitumor efficacy is assessed in PD-1-treatment-naive head and neck squamous cell cancer (HNSCC), non-MSI-H or pMMR colorectal cancer (CRC), PD-1-treatment-failure HNSCC, and PD-1/PDL-1 treatment naïve gastric cancer. Part B also assesses the safety and antitumor efficacy of Ab6 (at the preliminary RP2D) administered in combination with pembrolizumab and mFOLFOX7 (up to 20 subjects) or FOLFIRI (up to 20 subjects) in subjects with microsatellite stable (MSS) PD-1-treatment-naive CRC that have received ≤1 prior line of therapy.

Cohort A enrolled subjects with non-MSI-H or pMMR CRC that are naïve to prior PD-1/PD-L1 therapy and that have progressed on all available standard-of-care therapies. Ab6 antitumor efficacy was tested as monotherapy (Arm 1), in combination with pembrolizumab (Arm 2A and 2C), and as a coformulation (Ab6A, Arm 5). Monotherapy Ab6 (Arm 1) was administered at a dose of 800 mg in up to 20 subjects. In Arm 2, up to 100 subjects are treated with the combination of 200 mg Ab6 plus pembrolizumab (Arm 2A), and approximately 40 subjects are treated with the combination of 800 mg Ab6 plus pembrolizumab (Arm 2C). Forty subjects in Cohort A are enrolled to assess the safety, PK, and preliminary efficacy of Ab6A, a co-formulated product of 800 mg Ab6 and pembrolizumab (Arm 5).

Cohort B enrolled subjects with non-MSI-H or pM_MR CRC naïve to prior PD-1/PD-L1 therapy that have progressed on ≤1 prior line of therapy. Cohort B tested the antitumor efficacy of Ab6 (800 mg) administered in combination with pembrolizumab and mFOLFOX7 (up to 20 subjects, Arm 3) or FOLFIRI (up to 20 subjects, Arm 4).

Cohort C enrolled subjects with HNSCC that are naïve to prior PD-1/PD-L1 therapy and have progressed after ≥1 prior line of chemotherapy. Subjects received 200 mg Ab6 in combination with pembrolizumab (Arm 2A) to evaluate antitumor efficacy.

Cohort D enrolled subjects with HNSCC that have progressed following prior anti-PD-1/PD-L1 therapy. Subjects received 200 mg Ab6 in combination with pembrolizumab (Arm 2A) to evaluate antitumor efficacy.

Cohort E enrolled subjects with gastric adenocarcinoma that are naïve to prior PD-1/PD-L1 therapy and that have progressed on ≥1 prior line of chemotherapy. Cohort E employed a randomized comparison of 2 doses of Ab6 (200 mg [Arm 2A] and 700 mg [Arm 2B]) in combination with a fixed dose of pembrolizumab. Additionally, if antitumor activity is observed in Arm 2 of Cohort E (≥8 of 40 subjects with an objective response, irrespective of dose) an additional 20 subjects with gastric cancer are enrolled to receive Ab6 (800 mg) monotherapy (Arm 1).

### Subject Inclusion Criteria

1. Part A—Have a histologically or cytologically confirmed metastatic solid tumor for which there is no available therapy that may convey clinical benefit.
   Part B—Have 1 of the following histologically or cytologically confirmed tumor types:
      a. Cohort A—CRC for Arm 1, Arm 2A, Arm 2C, and Arm 5: CRC originating in either the colon or rectum that is locally advanced unresectable or metastatic (ie, Stage IV) and that has received, and progressed on, all available standard-of-care therapies including fluoropyrimidine, oxaliplatin, and irinotecan but has not been treated with prior anti-PD-1/PD-L1 therapy.
      b. Cohort B—CRC for Arm 3 and Arm 4: CRC originating in either the colon or rectum that is locally advanced unresectable or metastatic (ie, Stage IV) and has been treated with ≤1 line of systemic therapy but has not been treated with prior anti-PD-1/PD-L1 therapy. Subjects eligible to receive EGFR-targeted therapy must have previously received this treatment in order to be eligible for the study.
      c. Cohort C and Cohort D- HNSCC that is considered incurable by local therapies. Subjects should have progressed after receiving platinum-containing systemic therapy. Systemic therapy given as part of multimodal treatment for locally advanced disease is allowed. The eligible primary tumor locations are oropharynx, oral cavity, hypopharynx, and larynx. Subjects may not have a primary tumor site of nasopharynx (any histology). Subjects enrolled in the PD-1-treatment-naive HNSCC cohort (Cohort C) may not have been treated with prior anti-PD-1/PD-L1 therapy.
         Subjects enrolled in the PD-1-treatment-failure HNSCC cohort (Cohort D) must be refractory to an FDA approved anti-PD-1/PD-L1 monoclonal antibody (mAb) as either monotherapy or in combination with other approved checkpoint inhibitors or other therapies according to their label, defined as (subjects must meet all of the following criteria):
         i. Have received at least 2 doses of anti-PD-1/PD-L1 mAb.
         ii. Have progressive disease after anti-PD-1/PD-L1 mAb defined according to RECIST 1.1. The initial evidence of PD is to be confirmed by a second assessment, no less than 4 weeks from the date of the first documented PD, in the absence of rapid clinical progression.
         iii. Have documented PD within 24 weeks of the last dose of anti-PD-1/PD-L1 mAb. Patients who were re-treated with anti-PD-1/PD-L1 mAb and patients who were on maintenance with anti-PD-1/PD-L1 mAb will be allowed to enter the trial as long as there is documented PD within 24 weeks of the last treatment date (with anti-PD-1/PD-L1 mAb).
      d. Cohort E—Adenocarcinoma of the stomach and/or gastric-esophageal junction (GEJ) that is considered inoperable and that has received, and progressed on, at least 1 prior chemotherapy regimen or HER2/neu-targeted approved therapy (if HER2/neu-positive). In both cases, subjects must not have been treated with prior anti-PD-1/PD-L1 therapy. Have measurable disease by irRECIST 1.1 criteria.

In Part A of the study, Ab6 given as monotherapy and in combination with pembrolizumab 200 mg was well tolerated and had a manageable safety profile across all doses tested. Dose escalation proceeded to the maximum dose of 700 mg without any DLTs.

Efficacy data is available for the subjects treated in Part A of the study, including 18 subjects treated with Ab6 monotherapy and 15 subjects treated with combination therapy. In Part A, subjects received one of 5 preselected Ab6 doses between 7 mg and 700 mg either alone or in combination with a 200 mg fixed dose of pembrolizumab. In subjects treated on the monotherapy Arm 1 at all doses, the ORR was 5.5%, with 1 subject with endometrial cancer (microsatellite stable) experiencing partial response. This subject received the 210 mg Ab6 dose. Stable disease was also observed in a patient with leiomyosarcoma and a patient with appendiceal cancer in Arm 1.

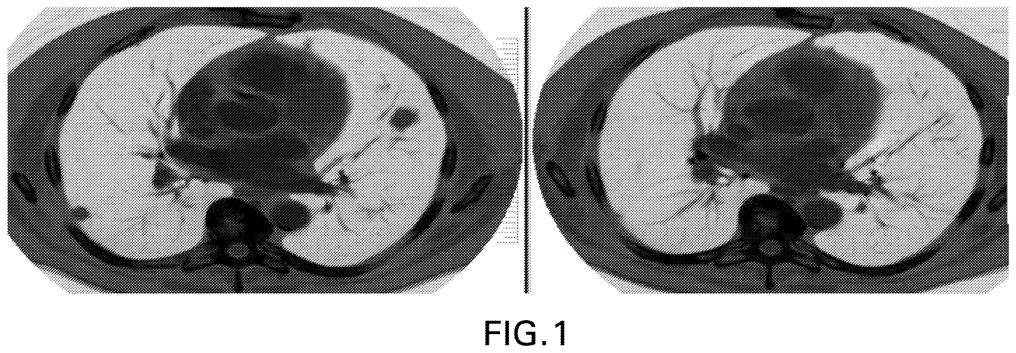

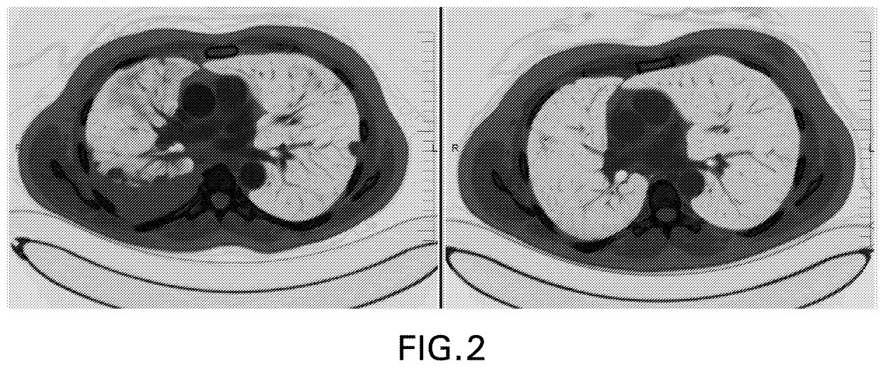

In subjects treated on the combination Arm 2 at all Ab6 doses, the objective response rate (ORR) was 26%, with 4 out of 15 subjects experiencing partial responses, 3 of which have been radiographically confirmed with a follow-up CT scan. The responders were diagnosed with colorectal cancer (microsatellite proficient) in 2 subjects at doses of 21 mg (FIGS. 1) and 70 mg Ab6, renal cell cancer in 1 subject at 7 mg Ab6 (FIG. 2), and fallopian tube cancer (BRCA negative) in 1 subject at 70 mg Ab6. Out of six colorectal cancer subjects treated on the combination arm in Part A, the ORR was 33%. A patient with adenocarcinoma of the GE junction received 70 mg Ab6 in combination with pembrolizumab and experienced a 28% reduction in target lesion size compared to baseline. Another patient with Amupllary cancer experienced stable disease.

Response rates in Part B subjects have demonstrated promising activity in a number of cohorts. In the Part B Non-MSI-H/pMMR CRC cohort, 4 out of 39 subjects experienced objective response (ORR and DCR of 10.2 and 25.6%, respectively). In comparison, pembrolizumab monotherapy activity is poor to absent in Non-MSI-H/pMMR CRC (O′Neil BH et al. PLoS One. 2017; 12(12), and regorafenib and TAS-102, approved agents in 3L CRC, which have an OS benefit of 2 months and ˜1-2% ORR. In the gastric cohort, 6 out of 46 subjects experienced objective response (ORR and DCR of 13 and 39%, respectively). 3 responses were at the 700 mg Ab6 dose, 3 responses were at the 200 mg Ab6 dose. This is in comparison to an ORR of 11.2% in third line gastric subjects treated on with pembrolizumab monotherapy (Fuchs, et al. Journal of Clinical Oncology 35, no. 15_suppl (May 20 2017) 4003-4003). In the PD-1 naive HNSCC cohort, 6 out of 23 subjects experienced objective response (ORR and DCR of 26.1 and 69.5%, respectively). Results from Keynote-055 pembrolizumab monotherapy demonstrated an objective response rate of 18% in PD-1 naive HNSCC patients that had progressed on at least 2 prior lines of therapy (Baumi J. et. al . Journal of Clinical Oncology 34 no.15 suppl_(May 20 2016) 6011-6011).

59 subjects with head and neck squamous-cell carcinoma (HNSCC) have been treated in Part B of the above Phase I study with the combination of 200 mg Q3W Ab6 and 200 mg Q3W pembrolizumab: 39 subjects with PD-1-treatment-naive HNSCC and 20 subjects with PD-1-treatment-failure HNSCC. The ORR (with confirmation) in subjects with PD-1-treatment-naive HNSCC was 12.8% (5 out of 39, 95% CI: 4.3, 27.4) and the DCR was 53.8% (21 out of 39, 95% CI: 37.2, 69.9). The ORR (without confirmation) in subjects with PD-1-treatment-naive HNSCC was 23.1% (9 out of 39, 95% CI: 11.1, 39.3) and the DCR was 56.4% (22 out of 39, 95% CI: 39.6, 72.2).The ORR (with confirmation) in subjects with PD-1-treatment-failure HNSCC was 0% (0 out of 20) and the DCR was 20.0% (4 out of 20, 95% CI: 5.7, 43.7). The ORR (without confirmation) in subjects with PD-1-treatment-failure HNSCC was 5% (1 out of 20, 95% CI: 0.1, 24.9) and the DCR was 25% (5 out of 20, 95% CI: 8.7, 49.1).

78 subjects with gastric cancer have been treated in Part B with the combination of Ab6 with 200 mg Q3W pembrolizumab: 39 at the 200 mg dose level of Ab6 and 39 at the 700 mg dose level of Ab6. The ORR (with confirmation) at the 200 mg dose level was 7.7% (3 out of 39, 95% CI: 1.6, 20.9) and the DCR was 23.1% (9 out of 39, 95% CI: 11.1, 39.3). The ORR (without confirmation) at the 200 mg dose level was 7.7% (3 out of 39, 95% CI: 1.6, 20.9) and the DCR was 25.6% (10 out of 39, 95% CI: 13.0, 42.1). The ORR (with confirmation) at the 700 mg dose level was 10.3% (4 out of 39, 95% CI: 2.9, 24.2) and the DCR was 33.3% (13 out of 39, 95% CI: 19.1, 50.2). The ORR (without confirmation) at the 700 mg dose level was 15.4% (6 out of 39, 95% CI: 5.9, 30.5) and the DCR was 35.9% (14 out of 39, 95% CI: 21.2, 52.8).

### Example 2

### Pharmacokinetic (PK) Studies of Ab6

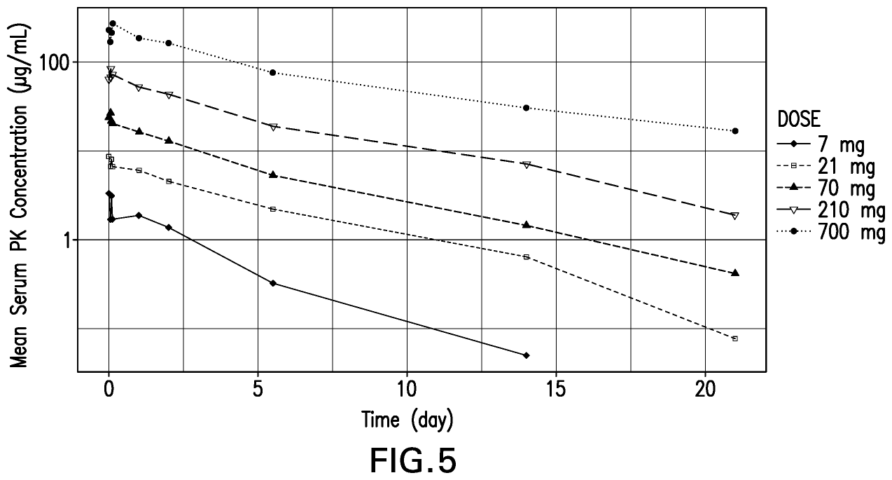

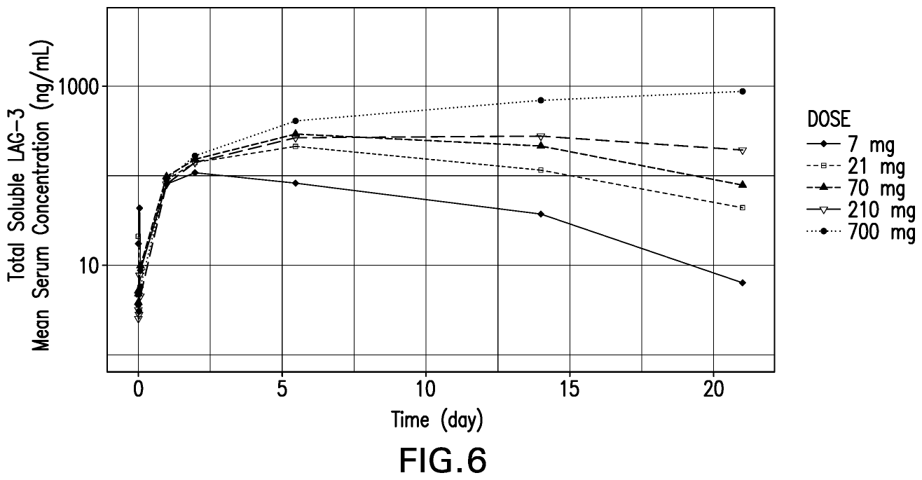

PK data from subjects treated during Part A of Ab6 (Ab6 alone and in combination with pembrolizumab) at doses from 7 mg to 700 mg showed that serum Ab6 exposures increased in a dose-dependent manner (FIG. 5). Blood samples from patients were collected on Days 1, 2, 8, 15 and 21 of Ab6 administration for PK analysis. PK profiles of Ab6 exposures suggest that target receptor mediated clearance of Ab6 is saturated at the 210 mg and 700 mg doses (FIG. 6).

Soluble (sLAG3) is a cleavage product of the membrane-bound LAG3 expressed on immune cells. Cleavage of LAG3 is required for optimal T- cell function (Goldberg and Drake, LAG-3 in Cancer Immunotherapy; Dranoff G. (eds) Cancer Immunology and Immunotherapy (2010); Current Topics in Microbiology and Immunology, vol 344. Springer, Berlin, Heidelberg). sLAG is detectable in serum in healthy patients, and to a greater extent, patients with cancer and chronic inflammatory disorders. sLAG3 was observed to increase in serum in a dose dependent manner following Ab6 administration in preclinical models. Ab6 binds both sLAG3 and membrane LAG3. If sLAG3 saturation is high, then membrane LAG3 saturation is also expected to be high. Therefore sLAG3 was chosen as a target engagement pharmacodynamic marker.

Data for the the target engagement pharmacodynamic marker sLAG3 from subjects treated during Part A of Ab6 (Ab6 alone and in combination with pembrolizumab) at doses from 7 mg to 700 mg also showed a dose-dependent increase in total soluble LAG-3 in serum, generally approaching saturation at the 210 mg and 700 mg doses (FIG. 6).

Compared to the antibody half-life predicted based on cynomolgus monkey PK parameters, Ab6 unexpectedly has a shorter half-life due to approximately three-fold faster clearance than predicted from cynomolgus monkey (predicted CLₕᵤₘₐₙ=0.168 L/day) or faster than anticipated clearance compared to typical monoclonal antibodies (Ryman, J. T., & Meibohm, B. (2017).

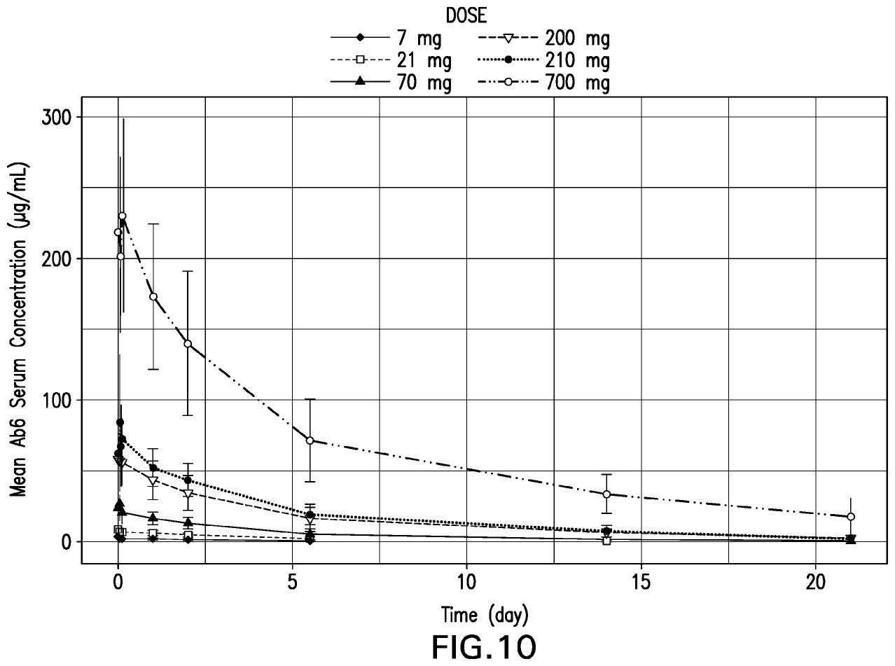

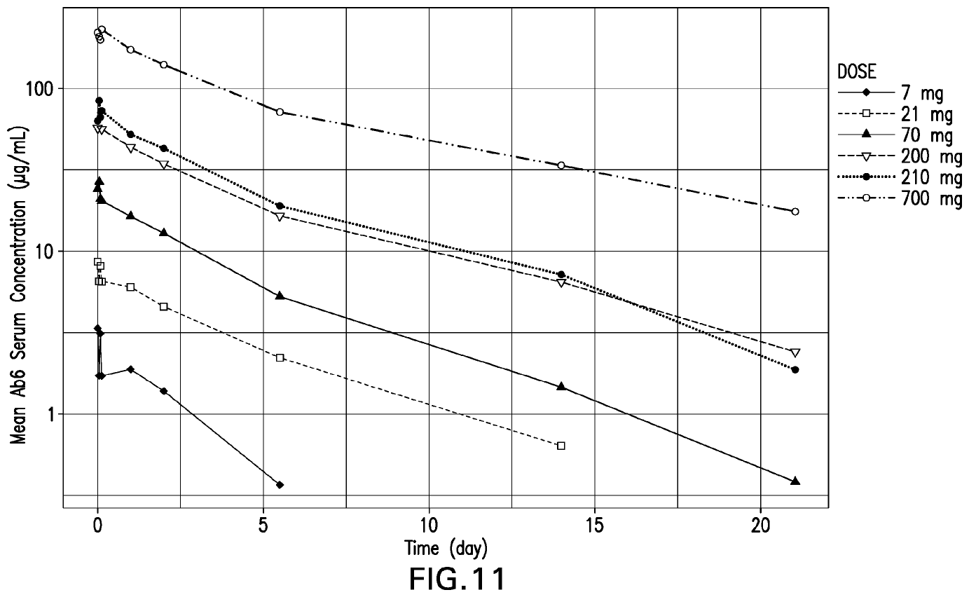

Pharmacokinetics of Monoclonal Antibodies. CPT: pharmacometrics & systems pharmacology, 6(9), 576-588). Preliminary PK analysis of the above phase I study exposures suggest that target receptor mediated clearance (reflecting target engagement of membrane LAG-3) of Ab6 was more likely to remain saturated at ≥700 mg dose taking into account PK (geometric CV >100%) C_{trough} variability observed in study subjects (FIGS. 10, 11 and 15).

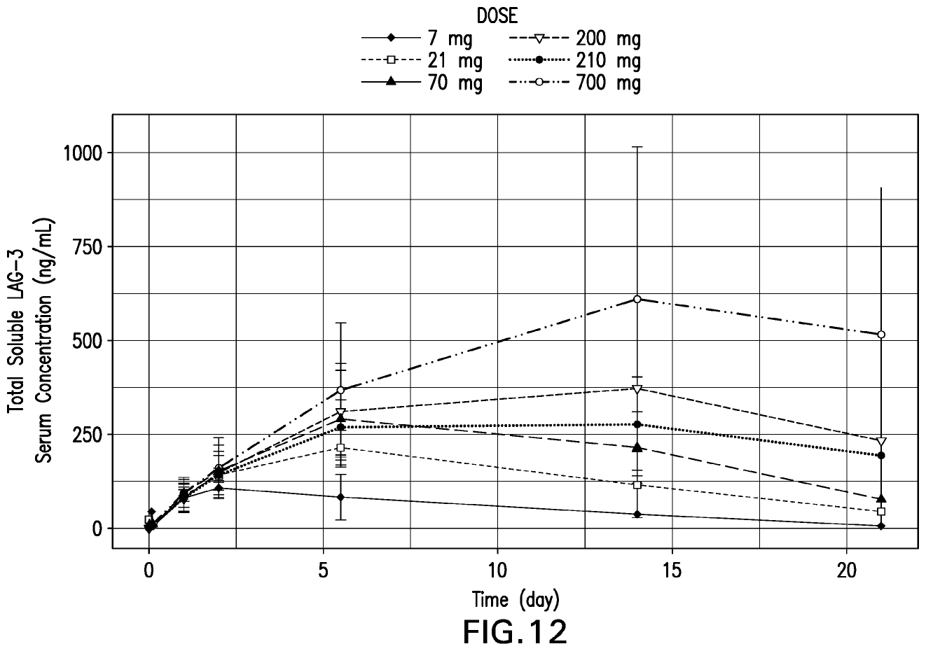

Data for the target engagement pharmacodynamic marker from subjects treated during Part A and Part B of the above phase I study demonstrate a dose-dependent increase in total sLAG-3 in serum (reflecting target engagement of sLAG-3) (FIG. 12). At the clinical 700 mg dose of Ab6, [sLAG3] plateaus throughout the dosing interval, suggesting that the effect of Ab6 is sustained throughout the dosing interval compared to lower doses.

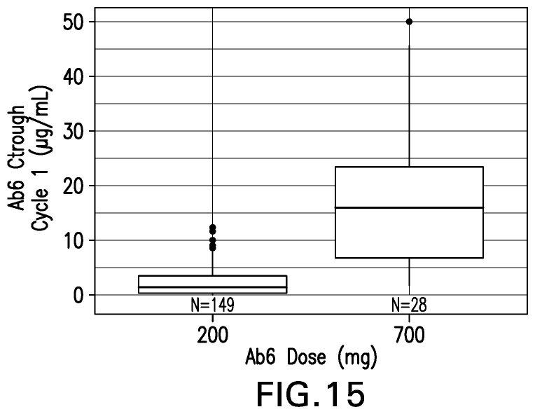

Furthermore, Ab6 PK exposures (Day 21 Ctrough) in 13% of patients at 200 mg are at Below limit of Quantitation (BLQ), and PK exposures (Day 21 Ctrough) of 0% of patients at 700 mg are at BLQ (See Table 6). BLQ was established according to J Pharm Biomed Anal. 2019 Jul 15;171:204-211. The high PK variability coupled with fast clearance of Ab6 results in patients reaching BLQ at the lower 200 mg level. In general, the Ctrough of the 700 mg dose was higher than the 200 mg dose (see FIG. 15).

TABLE 5
Ab6 Serum C_{trough} and variability on Day 21

| | | Geometric Mean | Geometric |
|---|---|---|---|
| Dose | N | (ug/mL) | CV % |
| 200 mg | 149 | 0.893 | 373 |
| 700 mg | 28 | 12.300 | 127 |

N = sample size

TABLE 6
Percent BLQ for Ab6 Serum C_{trough} on Day 21

| | Total | |
|---|---|---|
| Ab6 Dose | Treated | BLQ |
| 200 mg | 149 | 19 |
| 700 mg | 28 | 0 |

Preliminary efficacy data from the dose comparison cohort in gastric cancer (Cohort E) in Part B also suggests a trend towards better efficacy at 700 mg Ab6 (highest tested dose). An interim analysis of the randomized dose-comparison in Cohort E (Ab6 200 mg vs 700 mg plus a fixed 200 mg dose of pembrolizumab) was performed. At the time of the analysis, 39 gastric cancer subjects per arm (78 total), had been treated with Ab6. The median follow-up time was 98 days. Though not statistically significant, these data demonstrated trends towards improved disease control at the higher dose, including an ORR of 5.3% (95% CI: 0.6, 17.7) versus 8.3% (95% CI: 1.8, 22.5), and mean change in target lesion size of 29.9 cm (95% CI: 10.2, 49.7) versus 6.4 cm (95% CI: 9.3, 22.1) for 200 mg and 700 mg Ab6, respectively. In addition, no significant difference in safety has been observed at the 200 mg dose in comparison to the 700 mg dose in the randomized dose comparison cohort in gastric cancer.

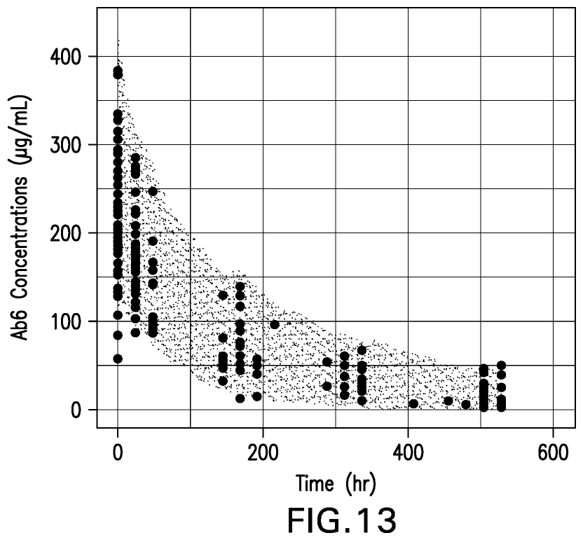

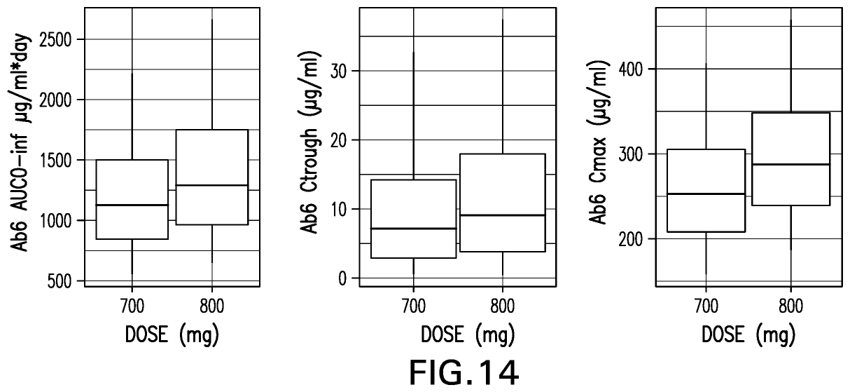

Based on a preliminary population PK analysis, predicted median Ab6 serum exposures at 800 mg are higher than 700 mg. However, the distribution of Ab6 serum concentrations at the 700 mg and 800 mg doses is expected to be similar, resulting in substantial overlap between the exposures at these 2 doses (FIGS. 13 and 14). Due to the predicted exposure overlap between 700 mg and 800 mg doses based on Ab6 drug concentrations, a similar safety profile for the 700 mg and 800 mg doses is anticipated.

### Example 3

### Measurement of PD-L1 and LAG3 Expression Levels

Specimens from non-MSI-H colorectal cancer, gastric and HNSCC patients of Part B were analyzed prior to treatment. Specimens for analysis are formalin-fixed and paraffin-embedded (FFPE) tissue sections. The IHC staining for PD-L1 expression was performed using the Dako Autostainer Link 48 platform (Dako AS480) and an automated staining protocol validated for the PD-L1 IHC 22C3 pharmDx assay according to US 2017/0285037, incorporated by reference in its entirety. The LAG-3 IHCAssay (LSBio, clone 17B4) was developed using 0.05 ug/ml of clone 17B4 from LSBio and validated on the Dako Autostainer Link 48 platform according to manufacturer's protocol. Formalin-fixed, paraffin-embedded 4-micron sections were used for the assay. Antigen retrieval was performed with the Envision FLEX Target Retrieval Solution, High pH (Agilent K800221-2) on the Dako PT link. The Agilent EnVision FLEX+, High pH (Link) (Agilent, K800221-2) was applied for the detection system. Stained slides were counterstained with Hematoxylin (Agilent, K8008) and cover slipped.

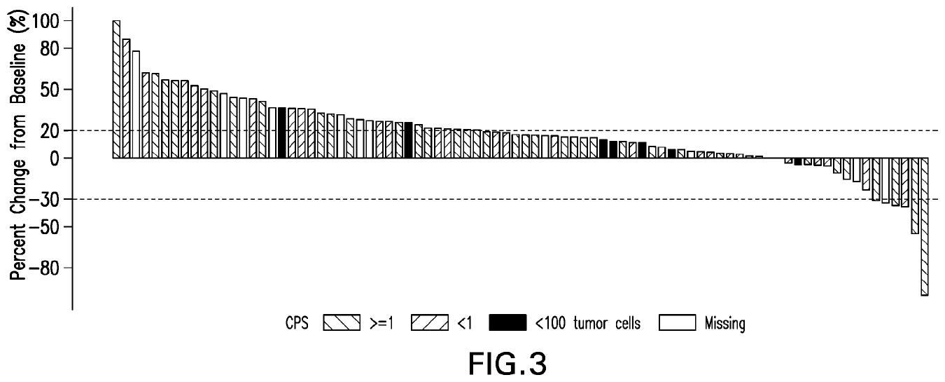

IHC data was collected for the entire CRC cohort of Part B for both PD-L1 expression and LAG3 expression. FIG. 3 shows that 54% of CRC tumors in this set using the CPS scoring system are PD-L1 positive. Of the PD-L1+tumors (CPS>=1%), 4 out of 46 are responders (9%). Three responders had CPS=1%, and 1 responder had CPS of 7%. Of the PD-L1−tumors (CPS<1%), 1 out of 35 was a responder (3%). Using a MIDS scoring system of at least 2, of the PD-L1+tumors, 4 out of 14 are responders (28%). Of the PD-L1−tumors with a MIDS score of less than 2, 0 out of 11 are responders (0%). Preliminary analysis of PDL 1 IHC using evaluation methods of TPS alone, MIDS alone or TPS+MIDS indicated that enrichment of the responder population was seen only with the MIDS alone and TPS+MIDS methods. Using a MIDS scoring system of at least 2, of the PD-L1+tumors, 4 out of 14 are responders (28%). Of the PD-L1−tumors with a MIDS score of less than 2, 0 out of 11 are responders (0%). This suggests that PDL-1 expression in inflammatory cells is an important component in predicting response to anti-LAG3 antibody and anti-PD-1 antibody treatment.

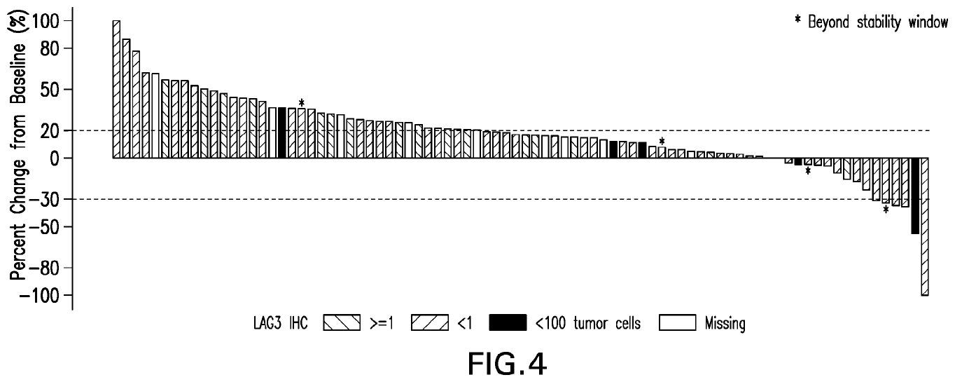

FIG. 4 shows LAG3 IHC results in CRC tumors. LAG 3 IHC was scored using the CPS-like % LAG3 positive cells. 20% of the CRC tumors in this set are LAG-3 positive. As shown in Table 7, 75% LAG3 positive CRC tumors are PD-L1 positive, while 29% PD-L1 positive CRC tumors are LAG3 positive. In other words, most LAG3 expressing CRC tumors also express PD-L1, but only a small number of PD-L1 expressing tumors also express LAG3.

TABLE 7
PD-L1 expression and LAG3
expression scores for CRC tumors

| PD-L1 | LAG3 | Prevalence | ORR |
|---|---|---|---|
| <1 | <1 | 40% | 3% |
| | | | (1/30) |
| | >=1 | 5% | 0% |
| | | | (0/4) |
| >=1 | <1 | 40% | 10% |
| | | | (3/30) |
| | >=1 | 16% | 0% |
| | | | (0/12) |

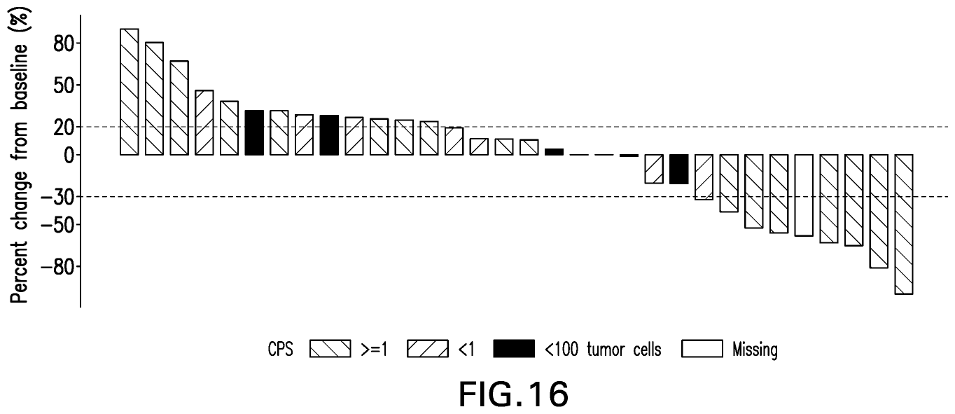

PD-L1 and LAG3 IHC data was collected for the gastric cancer expansion cohort administered with 700 mg Ab6 and 200 mg pembrolizumab. FIG. 16 shows that 65% of gastric tumors in this set using the CPS scoring system are PD-L1 positive. Of the PD-L1+tumors (CPS>=1%), 7 out of 22 are responders (32%). Of the PD-L1−tumors (CPS<1%), none were responders. PD-L1 IHC CPS has an AUROC (95% CI) of 0.90 (0.75, 1). The Area under the Receiver Operating Characteristic (AUROC) is a common summary statistic for the goodness of a predictor in a binary classification task. The ROC curve is created by plotting the true positive rate (TPR) against the false positive rate (FPR) at various threshold settings. The ROC is a probability curve and AUC represents the degree or measure of separability. An excellent model has an AUC close to 1.

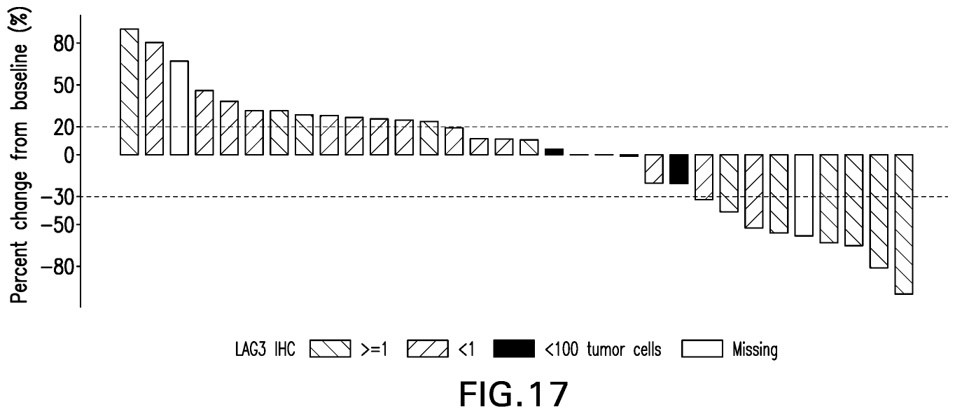

FIG. 17 shows that 42% of gastric tumors in this set using the CPS-like scoring system are LAG3 positive. Of the LAG3 + tumors (CPS-like>=1%), 6 out of 14 are responders (43%). Of the LAG3−tumors (CPS-like <1%), 1 out of 19 was a responder (5%). LAG3 IHC has an AUROC (95% CI) of 0.79 (0.62, 0.96). 93% LAG3 positive tumors are PD-L1 positive, and 65% PD-L1 positive tumors are LAG3 positive (See Table 8). In other words, almost all LAG3 positive tumors also express PD-L1, while only two-thirds of PD-L1 positive tumors also express LAG3.

TABLE 8
PD-L1 expression and LAG3 expression
scores for gastric tumors

| PD-L1 | LAG3 | Prevalence | ORR |
|---|---|---|---|
| <1 | <1 | 32% | 0% |
| | | | (0/10) |
| | >=1 | 3% | 0% |
| | | | (0/1) |
| >=1 | <1 | 23% | 14% |
| | | | (1/7) |
| | >=1 | 42% | 46% |
| | | | (6/13) |

Table 9 shows the distribution of PD-L1 and LAG3 IHC scores among the responders in this gastric cohort. Six out of seven responders express high levels of PD-L1 suggesting a higher cutoff (CPS≥10%) for patient selection. Similarly, for LAG3 IHC, the data suggests a cutoff of CPS≥1% provides enrichment in response rates.

TABLE 9
PD-L1 expression and LAG3 expression
scores for all responders in gastric tumors

| Patient | Response | PD-L1 IHC CPS | LAG3 IHC |
|---|---|---|---|
| 201206 | PR | 1 | 5 |
| 201222 | PR | 85 | 5 |
| 201242 | PR | 80 | 15 |
| 201247 | irPR | 75 | 3 |
| 201256 | PR | 80 | 5 |
| 201275 | PR | 50 | 3 |
| 201276 | PR | 35 | <1 |
| 201278 | PR | | |

Table 10 shows the clinical utility profile of the PD-L1 IHC assay at different CPS cut points in the Gastric cohort, where PPV is positive predictive value (the percentage of patient samples called “positive” according to the selected CPS cut point that are responders) and NPV is negative predictive value (the percentage of patient samples called “positive” according to the selected CPS cut point that are non-responders). Sensitivity is defined as the (.)/0 of responders that are positive according to the selected CPS cut point and specificity is defined as of non-responders that are negative according to the selected CPS cut point. As the CPS cut point increases, the prevalence decreases but PPV increases and NPV decreases. Sensitivity is maintained at al I cut points with CPS≥71% and specificity increases. The clinical utility profile of Table 10 also supports the CPS≥10% cutoff.

TABLE 10

| CPS Cut- | | | | | |
|---|---|---|---|---|---|
| point | Prevalence | PPV | Sensitivity | NPV | Specificity |
| 1 | 63.6 | 33.3 | 100 | 100 | 46.2 |
| 5 | 54.5 | 33.3 | 85.7 | 93.3 | 53.8 |
| 10 | 30.3 | 60 | 85.7 | 95.7 | 84.6 |
| 20 | 27.3 | 66.7 | 85.7 | 95.8 | 88.5 |
| 30 | 24.2 | 75 | 85.7 | 96 | 92.3 |

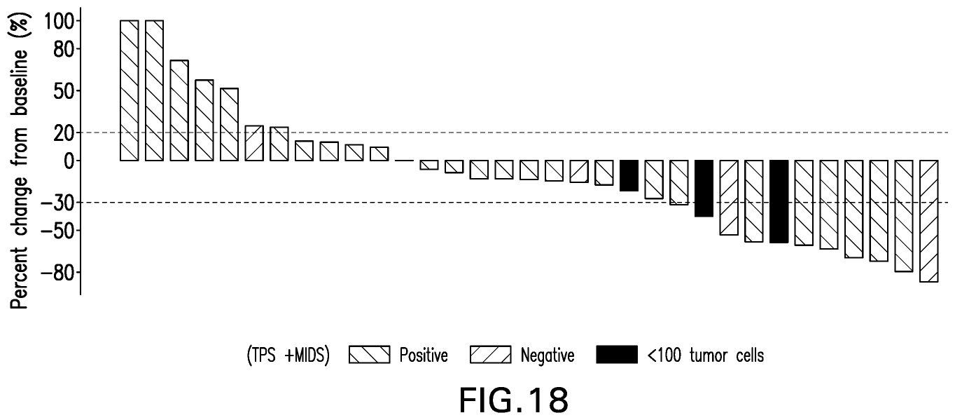

PD-L1 and LAG3 IHC data was collected in PD-1 naïve HNSCC patients administered with 200 mg Ab6 and 200 mg pembrolizumab. FIG. 18 shows that 86% of HNSCC tumors in this set using the exploratory TPS+MIDS scoring system are PD-L1 positive. In this scoring system if the TPS score is >1 or the MIDS score is >2, a tumor is considered positive. Of the PD-L1+tumors, 6 out of 30 are responders (20%). Of the PD-L1−tumors 2 out of 5 were responders (40%) (Table 11).

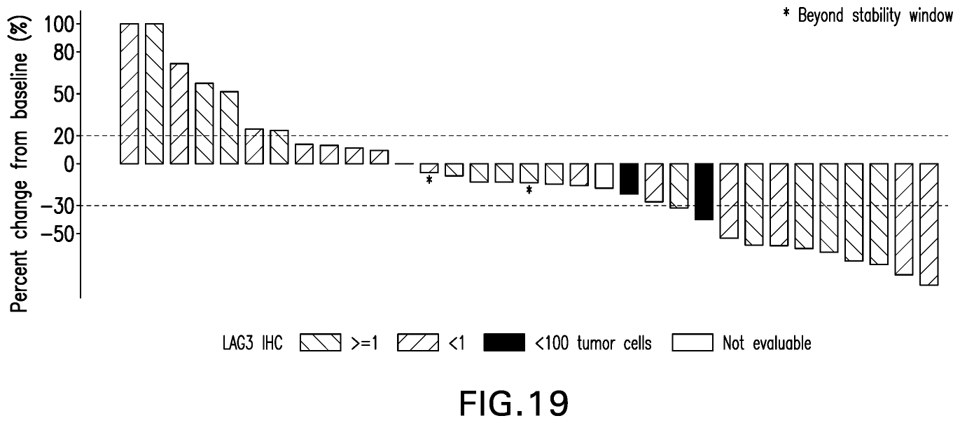

FIG. 19 shows that 49% of the tumors in the above HNSCC patients (using the % LAG3 positive cells scoring system) are LAG3 positive. Of the LAG3 +tumors 5 out of 17 are responders (29%). Of the LAG3−tumors 4 out of 14 are responders (22%). 100% LAG3 positive tumors are PD-L1 positive, and 59% PD-L1 positive tumors are LAG3 positive (See Table 11). In other words, all LAG3 positive tumors also express PD-L1, while only two-thirds of PD-L1 positive tumors also express LAG3.

TABLE 11
PD-L1 expression and LAG3 expression
scores for PD-1 naïve HNSCC tumors

| PD-L1 | LAG3 | Prevalence | ORR |
|---|---|---|---|
| <1 | <1 | 15% | 40% |
| | | | (2/5) |
| | >=1 | 0% | NA |
| >=1 | <1 | 35% | 8% |
| | | | (1/12) |
| | >=1 | 50% | 29% |
| | | | (5/17) |

### Example 4

### Clinical Studies of Anti-LAG3 Antibody in Advanced NSCLC

This is a group-sequential, adaptive randomization, multi-site, open-label study of pembrolizumab (MK-3475) at 200 mg Q3W IV infusion in combination with Ab6 at 200 mg Q3W IV infusion in study participants with advanced NSCLC who have not received prior systemic therapy for advanced disease and for whom an FDA approved targeted therapy (eg, erlotinib, crizotinib, etc.) is not indicated as first-line (1L) therapy based on defined oncogenic mutation (nonsquamous NSCLC only).

Participants are eligible to be included in the study only if all of the following criteria apply:

1. Have a histologically or cytologically confirmed diagnosis of Stage IV (American Joint Committee on Cancer [AJCC] v. 8) NSCLC and study participants should not have had prior systemic therapy for advanced disease.

2. Have confirmation that epidermal growth factor receptor—(EGFR), anaplastic lymphoma kinase—(ALK), c-ros oncogene 1 (ROS1), or B isoform of rapidly accelerated fibrosarcoma (B-Raf) directed therapy is not indicated as primary therapy (documentation of absence of tumor activating EGFR or B-Raf mutations and absence of ALK or ROS1 gene rearrangements). If participant's tumor is known to have a predominantly squamous histology, molecular testing for EGFR mutation and ALK and ROS1 translocations will not be required, as this is not part of current diagnostic guidelines.

3. Have measurable disease per RECIST 1.1 as assessed by the local site investigator/radiology. Lesions situated in a previously irradiated area are considered measurable if progression has been demonstrated in such lesions.

### Example 5

### Clinical Studies of Anti-LAG3 Antibody in Hematological Cancer

This is a nonrandomized, multi-site, open-label study of anti-LAG3 antibody Ab6 at doses 100, 200 or 700 mg Q3W IV infusion in combination with pembrolizumab (MK-3475) 200 mg Q3W IV infusion in participants with PD-1/L1-naive relapsed or refractory (R/R) classical Hodgkin lymphoma (cHL) (Cohort 1), PD-1/L1-refractory R/R cHL (Cohort 2), R/R diffuse large B-cell lymphoma (DLBCL) (Cohort 3), and R/R- indolent non-Hodgkin lymphoma (iNHL), with at least 10 participants in the R/R-iNHL group having follicular lymphoma.

### Patient Inclusion Criteria

Participants are Eligible to be Included in the Study only if all of the Following Criteria Apply:
1. Must have measureable disease, defined as at least 1 lesion that can be accurately measured in 2 dimensions with diagnostic quality cross sectional anatomic imaging (CT or MRI). Minimum measurement must be ≥15 mm in the longest diameter or ≥10 mm in the short axis.
2. Be able to provide a core or excisional tumor biopsy for biomarker analysis from an archival or newly obtained biopsy (within 3 months) at Screening.

### PD-1/L1-Naive R/R cHL (Cohort 1)

1. Must have histologically confirmed classical Hodgkin lymphoma.
2. Have relapsed (defined as disease progression after most recent therapy) or refractory (defined as failure to achieve CR or PR to most recent therapy) cHL and meet at least 1 of the following inclusions:
   a. Have failed to achieve a response or progressed after auto-SCT. Participants must have relapsed after treatment with or failed to respond to brentuximab vedotin post auto-SCT.
   b. Were unable to achieve a CR or PR to salvage chemotherapy and did not receive auto-SCT. Participants must have relapsed after treatment with or failed to respond to brentuximab vedotin.
   c. Participants who are ineligible for brentuximab vedotin, who discontinued brentituximab vedotin due to toxicity, or who reside in a region where brentuximab is not approved or available are eligible for the study.
3. Have not previously been treated with an anti-PD-1 or anti-PD-L1 therapy.

### PD-1/L1-Refractory R/R cHL (Cohort 2)

1. Must have histologically confirmed classical Hodgkin lymphoma.
2. Have relapsed (defined as disease progression after most recent therapy) or refractory (defined as failure to achieve CR or PR to most recent therapy) cHL and meet 1 of the following inclusions:
   a. Have failed to achieve a response or progressed after auto-SCT. Participants must have relapsed after treatment with or failed to respond to brentuximab vedotin post auto-SCT.
   b. Were unable to achieve a CR or PR to salvage chemotherapy and did not receive auto-SCT. Participants must have relapsed after treatment with or failed to respond to brentuximab vedotin.
   c. Participants who are ineligible for brentuximab vedotin, who discontinued brentituximab vedotin due to toxicity, or who reside in a region where brentuximab is not approved or available are eligible for the study.
3. Have progressed on treatment with an anti-PD-1/L1 mAb administered either as monotherapy or in combination with other checkpoint inhibitors or other therapies. PD-1 treatment progression is defined by meeting all of the following criteria:
   a. Have received at least 2 doses of an anti-PD-1 mAb that has been approved in Hodgkin's lymphoma, with the agent administered at the approved dose and schedule.
   b. Have demonstrated disease progression after PD-1/L1 as defined by Lymphoma Disease Response criteria (Cheson et al. Revised Response Criteria for Malignant Lymphoma. J Clin Oncol. 2007; 25:579-586.).
   c. Progressive disease has been documented within 12 weeks from the last dose of anti-PD-1/L1 mAb.
4. Have submitted pretrial imaging.

### R/R DLBCL (Cohort 3)

1. Have a histologically confirmed diagnosis of DLBCL. Transformed DLBCL, Gray zone lymphoma, Double hit lymphoma, and Primary mediastinal B cell lymphoma (PMBCL) are permitted.
2. Must have progressed following at least 2 lines of previous therapy, including progression after an autologous SCT, have declined SCT, or are not a candidate (per institutional criteria) for an autologous SCT. Participants who are ineligible for standard treatment or who have withdrawn from standard treatment before disease progression due to unacceptable toxicity warranting discontinuation of that treatment and precluding retreatment with the same agent will also be eligible.

### R/R-iNHL (Cohort 4)

1. Have histologically confirmed diagnosis of indolent (low-grade) B-cell lymphoma, defined as FL, marginal zone lymphoma, mucosa-associated lymphoid tissue lymphoma, or small lymphocytic lymphoma. Lymphoplasmacytic lymphomas, Waldenstrom's macroglobulinema, chronic lymphocytic leukemia (not associated with small lymphocytic lymphoma), and T-cell lymphomas are not eligible. At least 10 participants must have FL.
2. Participants must have progressed following at least 2 lines of previous therapy, which may include an autologous SCT. Participants who are ineligible for standard treatment or who have withdrawn from standard treatment due to unacceptable toxicity warranting discontinuation of that treatment and precluding retreatment with the same agent before progression of disease are also eligible.

### Safety Lead-in Phase

a. At least 14 participants are enrolled (at least 3/cohort) in the Safety Lead-in phase. Participants will receive pembrolizumab (at a fixed dose of 200 mg) in combination with Ab6 (at a starting dose of 200 mg) Q3W.
b. A modified Toxicity Probability Interval (mTPI) design [Ji, Y. and Wang, S.-J. 2013] is used to establish the recommended Phase 2 dose (RP2D) of Ab6 combination with pembrolizumab). Data from participants are monitored for the occurrence of DLTs beginning with the first cycle and continuously thereafter. Aggregate data are assessed at 6-month intervals. Lower and/or higher doses of Ab6 are explored depending on the combined safety, PK, and pharmacodynamics data available at each dose level.
c. If required by the mTPI design, the dose of Ab6 is lowered to 100 mg, and up to an additional 14 participants at this dose level is evaluated. Other lower Ab6 doses may be explored depending on the totality of the data for determination of the dose.
d. Higher Ab6 dose(s), up to 700 mg, may be explored based on the totality of the efficacy/PK and safety data of these Safety Lead-in phase participants. Additional participants may be enrolled if needed to assess efficacy.
e. The Safety Lead-in phase ends after 14 participants have been treated at any of the selected doses (which may include the optional doses). The pool adjacent-violators algorithm [Ji, Y. and Wang, S.-J. 2013] are used to estimate the DLT rates across doses in each arm under the assumption of monotonicity between DLT rates and dose levels. The dose with an estimated DLT rate closest to 30% is treated as a preliminary RP2D.
f. For participants treated at the preliminary RPTD dose in the Safety Lead-in phase, efficacy and safety data is combined with that of the corresponding Efficacy Expansion cohort. For participants treated at other doses than the confirmed dose, their data is not to be combined with the corresponding Efficacy Expansion phase cohort.

### Efficacy Expansion Phase

a. The Efficacy Expansion phase is enrolled approximately 120 participants overall, with approximately 30 participants in each of the 4 cohorts (these sample sizes include the participants in the Safety Lead-in phase). At least 10 participants in the R/R-iNHL cohort (Cohort 4) in the Efficacy Expansion phase must have FL.
b. In the Efficacy Expansion phase, an interim analysis for safety and an efficacy interim analysis for futility is conducted for each cohort after 12 participants have been enrolled (counting those participants from the Safety Lead-in phase in the particular cohort) and the last participant has completed the first response assessment, or otherwise discontinued study intervention.
c. After the RP2D is established, Cohorts 1 and 2 in the Efficacy Expansion phase is opened to enrollment first (see Safety Lead-in phase above). After the first 12 participants are enrolled in the respective cohort, an efficacy assessment is performed in that cohort. Enrollment in Cohorts 1 and 2 continue during this assessment. If a ≥50% ORR (≥6 of 12 participants; Cohort 1 efficacy target) is achieved in Cohort 1 or a ≥8.3% ORR (≥1 of 12 participants; Cohort 2 efficacy target) is achieved for Cohort 2, then enrollment is expanded to approximately 30 participants in the respective cohort. If either Cohort 1 or 2 achieves its efficacy target then, Cohorts 3 and 4 is opened to enrollment.
d. After the first 12 participants are enrolled in either Cohort 3 or 4, an efficacy analysis is performed in that respective cohort. Enrollment continues during this assessment. If either cohort achieves a ≥16.7% ORR (≥2 of 12 participants), then enrollment is expanded to approximately 30 participants in that cohort.

### Example 6

### A six-weekly (Q6W) Dosing Schedule for Pembrolizumab Across Multiple Tumor Types Based on an Evaluation using Modeling and Simulation

Pembrolizumab, an anti-PD-1 checkpoint inhibitor currently approved for use in multiple cancer indications, has demonstrated safety and efficacy when administered at a dose of either 200 mg or 2 mg/kg Q3W. An alternative extended dosing regimen would provide the benefits of convenience and flexibility to both patients and prescribers. The robust characterization of pembrolizumab pharmacokinetics (PK) and exposure (concentration)-response (E-R) relationships for both efficacy and safety allow the use of model-based approaches to support alternative dosing regimens for pembrolizumab.

The dose for a Q6W schedule of pembrolizumab was selected by matching exposures with the approved Q3W (200 mg and 2 mg/kg) regimens after PK steady state is achieved; the efficacy and safety between regimens were bridged based on knowledge of E-R. PK exposures were simulated up to 24 weeks of dosing, to ensure steady state in all subjects, using the established population PK model (with time dependent elimination) of pembrolizumab that adequately described PK across multiple tumor types. Efficacy was bridged using exposure metrics at steady state, AUCss or time-averaged concentration (Cavg,ss) and trough concentrations (Cmin,ss), which were compared between regimens. The safety profile of pembrolizumab at the Q6W schedule was bridged by ensuring that the predicted peak concentrations at steady state (Cmax,ss) are below those of the maximum clinically administered and well-tolerated dose of 10 mg/kg Q2W.

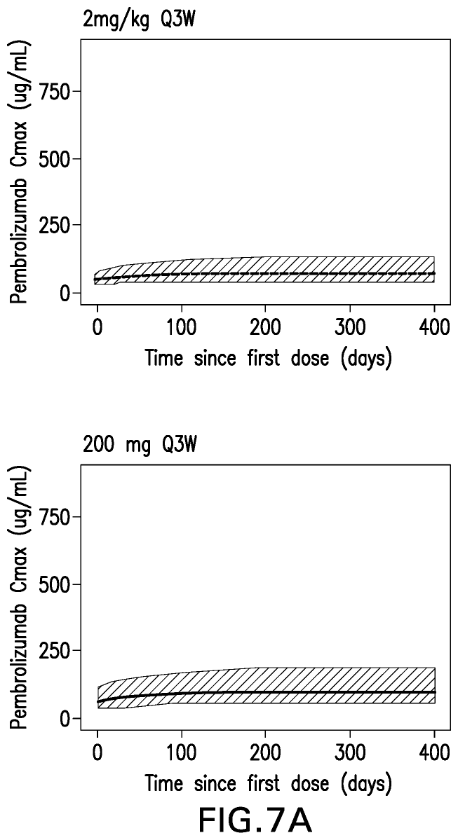

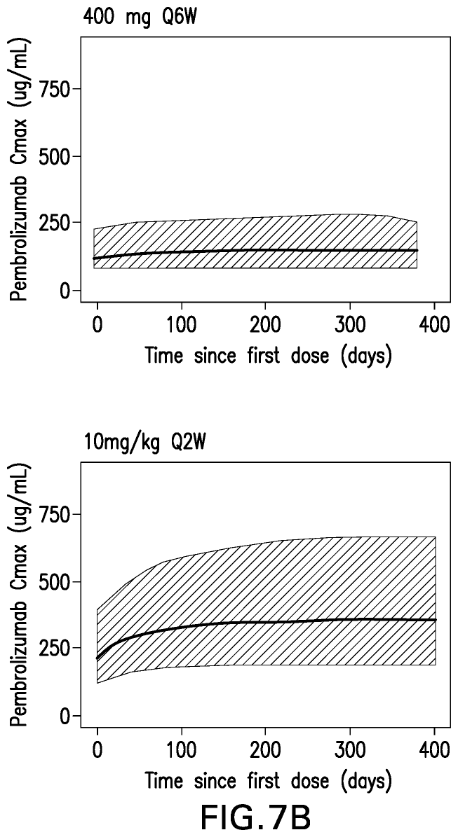

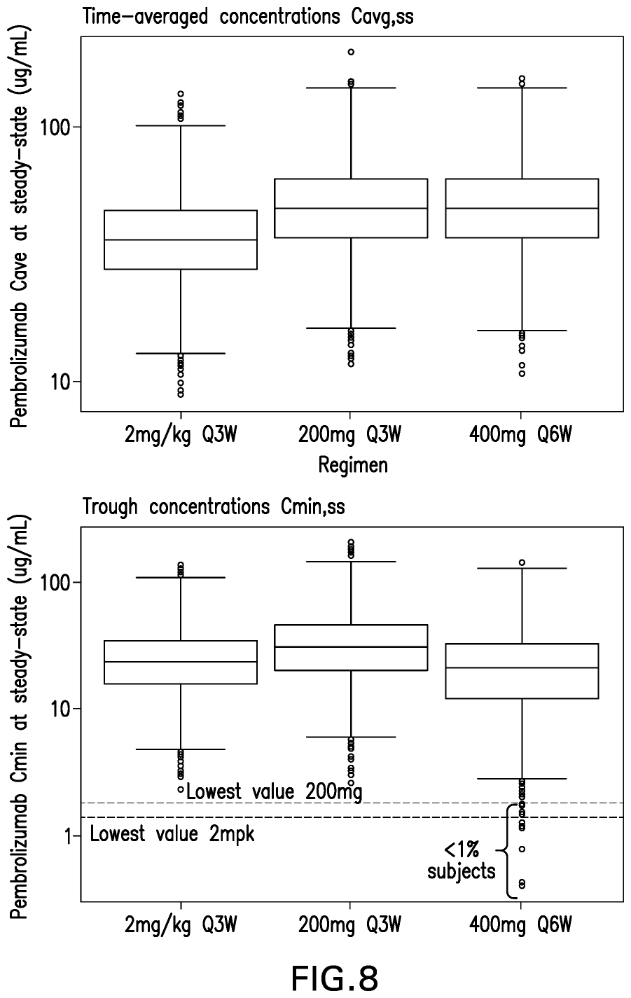

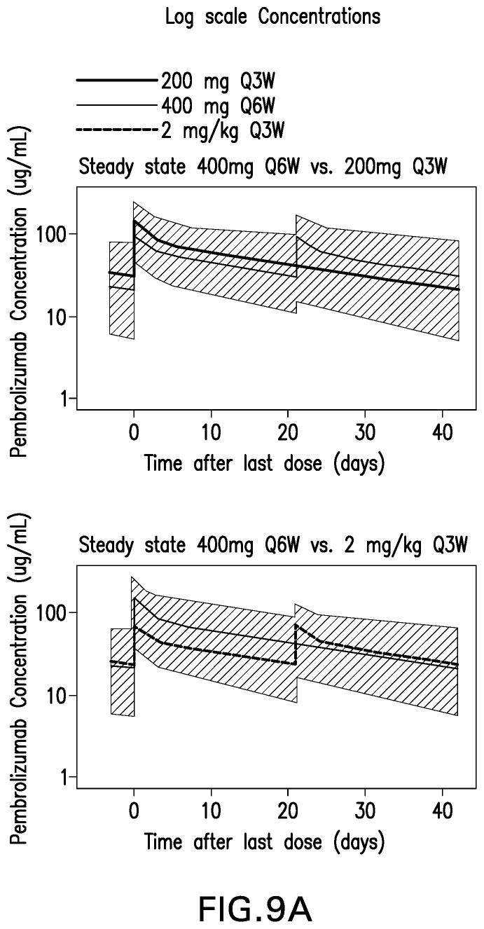

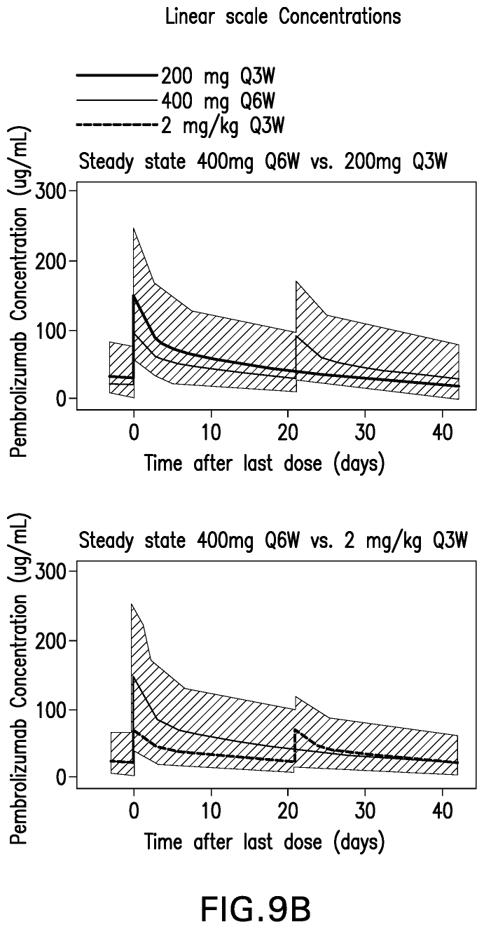

The PK of pembrolizumab after administration of 400 mg Q6W is predicted to follow a similar profile as the PK at the approved 200 mg Q3W and 2 mg/kg Q3W dosing regimens (see FIG. 8). The exposure metrics as compared between regimens are summarized in Table 12. The 400 mg Q6W dosing regimen of pembrolizumab was selected based on similar predicted exposures (Cavg,ss or AUCss, geometric mean (GM) ˜1% higher) compared with those achieved at 200 mg Q3W (see FIG. 7). Less than 1% subjects were predicted to have Cmin,ss that are lower in comparison with those at 200 mg Q3W and 2 mg/kg Q3W (FIG. 8). The predicted Cmax,ss for 400 mg Q6W are well below (GM ˜65% lower) that achieved with 10 mg/kg Q2W, which has been shown to have acceptable safety across multiple tumor types (see FIG. 7). Given the similar exposure profiles and the established, flat E-R relationships for pembrolizumab at clinically tested doses, the clinical outcomes achieved with 400 mg Q6W are expected to be similar to those with 200 mg Q3W across tumor types.

Based on the modeling and simulation approach used herein, it is expected that a 400 mg Q6W dosing regimen for pembrolizumab would lead to PK exposures that are similar to the approved 200 mg Q3W and 2 mg/kg dosing regimens. PK simulations demonstrate that in terms of pembrolizumab exposures—Average concentration over the dosing interval (Cavg) (or area under the curve [AUC]) at 400 mg Q6W was similar to that at the approved 200 mg Q3W dose, thus bridging efficacy between dosing regimens. Trough concentrations (Cmin) at 400 mg Q6W were generally within the range of those achieved with 2 mg/kg or 200 mg Q3W in the majority (>99%) of patients. Peak concentrations (Cmax) at 400 mg Q6W were well below the Cmax for the highest clinically tested dose of 10 mg/kg Q2W, supporting that the safety profile for 400 mg Q6W should be comparable to the established safety profile of pembrolizumab. Exposure-response (E-R) for pembrolizumab was demonstrated to be flat across indications, and OS predictions in melanoma and NSCLC demonstrate that efficacy at 400 mg Q6W is expected to be similar to that at 200 mg or 2 mg/kg Q3W, given the similar exposures; thus 400 mg Q6W is expected to be efficacious across indications.

TABLE 12
Summary of Pembrolizumab PK Exposure
Metrics for the 400 mg Q6W Dosing
Regimen Based on Simulations

| | Q6W |
|---|---|
| Alternative Dosing Regimen | 400 mg |
| Cavg, ss | |
| Relative to 200 mg Q3W, | 0.7% |
| % difference in GM at steady state | |
| Cmin, ss | |
| Relative to 2 mpk Q3W, | −12.6% |
| % difference in GM at steady state | |
| % of patients below lower limit | <1% |
| of range for 200 mg and | |
| 2 mpk Q3W at steady state | |
| Cmax, ss | |
| Relative to 10 mpk Q2W, | −65.6% |
| % difference in GM at steady state | |

### Example 7

### A Phase 1 Randomized Clinical Study of Pembrolizumab to Evaluate the Safety and Tolerability of Intravenous Infusion of 400 mg Pembrolizumab Q6W in Participants with Advanced Melanoma

This study is designed to assess the pharmacokinetics (PK), safety and tolerability of pembrolizumab when administered every 6 weeks (Q6W). A cohort of 100 participants are given 400 mg pembrolizumab Q6W. PK, efficacy, and safety data are collected from this cohort of participants. Male/female participants of at least 18 years of age with advanced melanoma are enrolled in the study. No stratification based on age, sex, or other characteristics is used in this study.

Participants receive IV infusion of 400 mg pembrolizumab Q6W from cycles 1 to 18. PK, efficacy, and safety data are collected from these participants. Results provide preliminary PK, efficacy, and safety data of pembrolizumab when administered Q6W. Based on the robust understanding of pembrolizumab clinical pharmacology and its well-established E-R profiles, such a dosing schedule change is expected to produce similar efficacy and safety in all treatment settings where 200 mg Q3W pembrolizumab is approved (including monotherapy and in combination with other agents). Thus, a 400 mg Q6W regimen would have a similar benefit-risk profile to 200 mg Q3W, as a less frequent dosing regimen in the clinical use of pembrolizumab based on modeling and simulation analyses.

### Study Design

The study, which is a randomized, cross-over, multicenter, open-label, safety study of pembrolizumab in participants with advanced melanoma, is conducted in conformance with Good Clinical Practices (GCP). This Phase 1 study is conducted in participants with unresectable or metastatic melanoma. The treatment period continues every 42 days for up to 18 cycles (approximately 2 years). Treatment will continue as long as participants are receiving benefit from treatment and have not had disease progression or met any criteria for study withdrawal. In greater detail, the study consists of: (1) A screening period of up to a 28-day duration to ensure that the participant is eligible for the study and (2) An intervention period of approximately 104 weeks of treatment with pembrolizumab. Participants receive pembrolizumab via IV infusion over 30 minutes Q6W for up to 18 cycles, and (3) A follow-up period during which participants are monitored for AEs for 30 days and serious adverse events (SAEs) for 90 days (30 days if the participant initiates new anticancer therapy). Participants with an ongoing AE at the time of treatment discontinuation are followed until resolution, stabilization, the event is otherwise explained, or the participant is lost to follow-up.

Participants who discontinue for reasons other than radiographic disease progression have post-treatment follow-up imaging for disease status until disease progression is documented radiographically per RECIST 1.1 and, when clinically appropriate, confirmed by the site per iRECIST, initiating a non-study cancer treatment, withdrawing consent, becoming lost to follow-up or the end of the study. All participants are followed by telephone for overall survival in the Survival follow-up period until death, participant withdrawal of consent, becoming lost to follow-up or the end of the study. Upon study completion, participants may be enrolled in a pembrolizumab extension study if available.

All participants enrolled into this study will have a diagnosis of advanced melanoma. The results of this study will contribute to an understanding of the PK characteristics of pembrolizumab when administered in a Q6W dosing regimen. Safety parameters commonly used for evaluating investigational systemic anticancer treatments are included as safety endpoints including, but not limited to, the incidence of, causality, and outcome of adverse events (AEs)/serious adverse events (SAEs); and changes in vital signs and laboratory values. AEs will be assessed as defined by National Cancer Institute Common Terminology Criteria for Adverse Events [NCI CTCAE] Version 4.0).

An objective of this trial is to characterize the PK profile of pembrolizumab following administration as an IV infusion Q6W. PK data is analyzed after all participants complete Cycle 5. PK parameters include AUC, Cmax, and Cmin. Formation of Antidrug Antibodies (ADA) can potentially confound drug exposures at therapeutic doses and prime for subsequent infusion-related toxicity. Antidrug antibody response to pembrolizumab at the beginning of each of Cycles 1, 2, 4, and 5 are determined. Any impact of presence of ADAs on exposure of pembrolizumab is explored.

This study uses ORR based on RECIST 1.1 criteria as assessed by blinded independent central review (BICR) as the primary endpoint . Objective response rate is an acceptable measure of clinical benefit for a late stage study that demonstrates superiority of a new antineoplastic therapy, especially if the magnitude of the effect is large and the therapy has an acceptable risk/benefit profile. The use of BICR and RECIST 1.1 to assess ORR is typically considered acceptable by regulatory authorities. Images are submitted to an imaging CRO (iCRO) and read by independent central review blinded to treatment assignment to minimize bias in the response assessments.

Overall survival (OS) is a secondary endpoint and has been recognized as the gold standard for the demonstration of superiority of a new antineoplastic therapy in randomized clinical studies. RECIST 1.1 is used by the BICR when assessing images for efficacy measures and by the local site when determining eligibility. Modified RECIST 1.1 for immune-based therapeutics (iRECIST) assessment has been developed and published by the RECIST Working Group, with input from leading experts from industry and academia, along with participation from the US Food and Drug Administration and the European Medicines Agency. The unidimensional measurement of target lesions, qualitative assessment of nontarget lesions, and response categories are identical to RECIST 1.1, until progression is seen by RECIST 1.1. However, if a participant is clinically stable, additional imaging may be performed to confirm radiographic progression. iRECIST is used by investigators to assess tumor response and progression and make treatment decisions as well as for exploratory efficacy analyses where specified.

### Inclusion Criteria

Participants are Eligible to be Included in the study only if all of the Following Criteria Apply:
Participant has histologically or cytologically confirmed diagnosis of advanced melanoma
Participant has unresectable Stage III or Stage IV melanoma, as per American Joint Committee on Cancer (AJCC) staging system not amenable to local therapy.
Participant is untreated for advanced or metastatic disease except as follows: BRAF V600 mutant melanoma may have received standard of care targeted therapy (eg, BRAF/MEK inhibitor, alone or in combination) and be eligible for this study
Prior adjuvant or neoadjuvant melanoma therapy is permitted if it was completed at least 4 weeks before randomization and all related AEs have either returned to baseline or stabilized (resolution of toxic effect(s) of the most recent prior therapy to Grade 1 or less [except alopecia]). If subject received major surgery or radiation therapy of >30 Gy, they must have recovered from the toxicity and/or complications from the intervention.

A female participant is eligible to participate if she is not pregnant, not breastfeeding, and agrees to follow specific contraceptive guidance during the treatment period and for at least 120 days or provides informed consent.

A participaent should have an Eastern Cooperative Oncology Group (ECOG) performance status 0 (fully active, able to carry on all pre-disease performance without restriction) or 1 (restricted in physically strenuous activity but ambulatory and able to carry out work of a light or sedentary nature, e.g., light house work, office work) and should have adequate organ function as defined in Table 13. Specimens are collected within 72 hours prior to the start of study intervention.

TABLE 13
Adequate Organ Function Laboratory Values

| | |
|---|---|
| System | Laboratory Value |

Hematological

| | |
|---|---|
| Absolute neutrophil count (ANC) | ≥1500/μL |
| Platelets | ≥100 000/μL |
| Hemoglobin | ≥9.0 g/dL or ≥ 5.6 mmol/L¹ |

Renal

| | |
|---|---|
| Creatinine OR | ≤1.5 × ULN OR |
| Measured or calculated² creatinine | ≥30 mL/min for participant with creatinine |
| clearance | levels > 1.5 × institutional ULN |
| (GFR can also be used in place of | |
| creatinine or CrCl) | |

Hepatic

| | |
|---|---|
| Total bilirubin | ≤1.5 × ULN OR direct bilirubin ≤ ULN for |
| | participants with total bilirubin levels > 1.5 × ULN |
| AST (SGOT) and ALT (SGPT) | ≤2.5 × ULN (≤5 × ULN for participants with liver |
| | metastases) |

Coagulation

| | |
|---|---|
| International normalized ratio (INR) OR | ≤1.5 × ULN unless participant is receiving |
| prothrombin time (PT) | anticoagulant therapy as long as PT or PTT is within |
| Activated partial thromboplastin time (aPTT) | therapeutic range of intended use of anticoagulants |

¹Criteria must be met without erythropoietin dependency and without packed red blood cell (pRBC) transfusion within last 2 weeks.
²Creatinine clearance (CrCl) should be calculated per institutional standard.
ALT (SGPT) = alanine aminotransferase (serum glutamic pyruvic transaminase);
AST (SGOT) = aspartate aminotransferase (serum glutamic oxaloacetic transaminase);
GFR = glomerular filtration rate;
ULN = upper limit of normal.

### Exclusion Criteria

Participants are Excluded from the Study if any of the Following Criteria Apply:
The participant is a woman of child-bearing potential (WOCBP) who has a positive urine pregnancy test within 72 hours prior to randomization or treatment allocation. If the urine test is positive or cannot be confirmed as negative, a serum pregnancy test is required.
The participant has received prior systemic treatment for unresectable or metastatic melanoma (except as noted in inclusion criteria described above).
The participant has received prior therapy with an anti-PD-1, anti-PD-L1, or anti-PD-L2 or with an agent directed to another stimulatory or co-inhibitory T-cell receptor (eg, OX-40 and CD137) or any other antibody or drug specifically targeting checkpoint pathways other than anti-CTLA-4 which is permitted in the adjuvant setting.
The participant has received prior radiotherapy within 2 weeks of start of study treatment. Participants must have recovered from all radiation-related toxicities, not require corticosteroids, and not have had radiation pneumonitis.
The participant has received a live vaccine within 30 days prior to the first dose of study drug. Examples of live vaccines include, but are not limited to, the following: measles, mumps, rubella, varicella/zoster (chicken pox), yellow fever, rabies, Bacillus Calmette-Guérin (BCG), and typhoid vaccine. Seasonal influenza vaccines for injection are generally killed virus vaccines and are allowed; however, intranasal influenza vaccines (eg, FluMist®) are live attenuated vaccines and are not allowed.
The participant is currently participating in or has participated in a study of an investigational agent or has used an investigational device within 4 weeks prior to the first dose of study intervention.
The participant has a diagnosis of immunodeficiency or is receiving chronic systemic steroid therapy (in dosing exceeding 10 mg daily of prednisone equivalent) or any other form of immunosuppressive therapy within 7 days prior the first dose of study drug.
The participant has a known additional malignancy that is progressing or has required active treatment within the past 2 years. Note: Participants with basal cell carcinoma of the skin, squamous cell carcinoma of the skin, or carcinoma in situ (eg, breast carcinoma, cervical cancer in situ) that have undergone potentially curative therapy are not excluded.
The participant has known active CNS metastases and/or carcinomatous meningitis. Participants with previously treated brain metastases may participate provided they are radiologically stable, (ie, without evidence of progression) for at least 4 weeks by repeat imaging (note that the repeat imaging should be performed during study screening), clinically stable and without requirement of steroid treatment for at least 14 days prior to first dose of study intervention.
The participant has severe hypersensitivity (>Grade 3) to pembrolizumab and/or any of its excipients.
The participant has ocular melanoma.
The participant has an active autoimmune disease that has required systemic treatment in past 2 years (ie, with use of disease modifying agents, corticosteroids or immunosuppressive drugs). Replacement therapy (eg, thyroxine, insulin, or physiologic corticosteroid replacement therapy for adrenal or pituitary insufficiency) is not considered a form of systemic treatment and is allowed.
The participant has a history of (non-infectious) pneumonitis that required steroids or has current pneumonitis.
The participant has an active infection requiring systemic therapy.
The participant has a known history of human immunodeficiency virus (HIV) infection.
The participant has a known history of Hepatitis B (defined as Hepatitis B surface antigen [HBsAg] reactive) or known active Hepatitis C virus (defined as HCV RNA [qualitative] is detected) infection.
The participant has a history or current evidence of any condition, therapy, or laboratory abnormality that might confound the results of the study, interfere with the participant's participation for the full duration of the study, or is not in the best interest of the participant to participate, in the opinion of the treating investigator.
The participant has a known psychiatric or substance abuse disorder that would interfere with cooperating with the requirements of the study.
The participant is pregnant or breastfeeding or expecting to conceive or father children within the projected duration of the study, starting with the screening visit through 120 days after the last dose of study intervention.

### Discontinuation of Study Intervention and Participant Withdrawal

Discontinuation of study intervention does not represent withdrawal from the study. As certain data on clinical events beyond study intervention discontinuation may be important to the study, they must be collected through the participant's last scheduled follow-up, even if the participant has discontinued study intervention. Therefore, all participants who discontinue study intervention prior to completion of the protocol-specified treatment period will still continue to participate in the study.

Participants may discontinue study intervention at any time for any reason or be dropped from the study intervention at the discretion of the investigator should any untoward effect occur. In addition, a participant may be discontinued from study intervention by the investigator if study intervention is inappropriate, the study plan is violated, or for administrative and/or other safety reasons.

A participant must be discontinued from study intervention but continue to be monitored in the study for any of the following reasons:
The participant or participant's legally acceptable representative requests to discontinue study intervention.
The participant interrupts study intervention administration for more than 12 consecutive weeks or has 3 cumulative missed doses.
The participant has a medical condition or personal circumstance which, in the opinion of the investigator, placed the participant at unnecessary risk from continued administration of study intervention.
The participant has a confirmed positive serum pregnancy test.
The participant has confirmed radiographic disease progression
The participant has any progression or recurrence of any malignancy, or any occurrence of another malignancy that requires active treatment
The participant has unacceptable adverse experiences.
The participant has intercurrent illness other than another malignancy as noted above that prevents further administration of treatment.
Investigator decides to discontinue treatment.
The participant has recurrent Grade 2 pneumonitis
The participant has completed 35 treatments (approximately 2 years) with pembrolizumab

A participant is withdrawn from the study if the participant or participant's legally acceptable representative withdraws consent from the study. If a participant withdraws from the study, they will no longer receive study treatment or be followed at scheduled protocol visits.

### Efficacy/Assessments

Tumor assessments include all known or suspected disease sites. Imaging may include chest, abdomen, and pelvis computed tomography (CT) or magnetic resonance imaging (MRI) at baseline and when disease progression or brain metastases is suspected. Tumor imaging is strongly preferred to be acquired by CT. For chest, abdomen and pelvis, contrast-enhanced MRI may be used when CT with iodinated contrast is contraindicated, or when mandated by local practice. For the brain, MRI is the strongly preferred imaging modality.

The same imaging modality technique (ideally the same scanner, and consistent use of contrast) is used in a participant throughout the study. Consistent use of imaging techniques will help to optimize the reproducibility of the assessment of existing and new tumor burden, and to improve the accuracy of the assessment of response or progression. All scheduled images for all study participants are reviewed by the investigator for disease progression. In addition, images (including those obtained via other modalities) that are obtained at an unscheduled time point to determine disease progression (as well as imaging obtained for other reasons, but that capture radiologic progression based on investigator assessment), are also be filed at the study site.

Confirmation of measurable disease based on RECIST 1.1 by BICR at screening will be used to determine participant eligibility. Confirmation by the BICR that the participant's imaging shows at least 1 lesion that is appropriate for selection as a target lesion per RECIST 1.1 is required prior to participant allocation.

### Initial Tumor Imaging

Initial tumor imaging at screening is performed within 28 days prior to the date of first dose. Any imaging obtained after Cycle 1 Day 1 of treatment is not included in the screening assessment.

The site study team reviews screening images to confirm the participant has measurable disease per RECIST 1.1. If brain imaging is performed to document the stability of existing metastases, MRI is used if possible. If MRI is medically contraindicated, CT with contrast is an acceptable alternative.

### Tumor Imaging During the Study

The first on-study imaging assessment is performed at 12 weeks (84 days±7 days]) from the date of first dose. Subsequent tumor imaging is performed every 9 weeks (63 days±7 days) or more frequently if clinically indicated. After 52 weeks (365 days±7 days), participants who remain on treatment will have imaging performed every 12 weeks (84 days±7 days).

Objective response is confirmed by a repeat imaging assessment. Tumor imaging to confirm PR or CR is performed at least 4 weeks after the first indication of a response is observed. Participants will then return to regular scheduled imaging, starting with the next scheduled imaging time point. Participants who receive additional imaging for confirmation do not need to undergo the next scheduled tumor imaging if it is less than 4 weeks later; tumor imaging may resume at the subsequent scheduled imaging time point.

Per modified iRECIST, disease progression is confirmed by the site 4 to 8 weeks after first radiologic evidence of progressive disease (PD) in clinically stable participants. Participants who have unconfirmed disease progression may continue on treatment at the discretion of the investigator until progression is confirmed by the site. Participants who receive confirmatory imaging do not need to undergo the next scheduled tumor imaging if it is less than 4 weeks later; tumor imaging may resume at the subsequent scheduled imaging time point, if clinically stable. Participants who have confirmed disease progression by iRECIST, as assessed by the site, will discontinue study treatment.

### End-of-Treatment and Follow-up Tumor Imaging

For participants who discontinue study intervention, tumor imaging is performed at the time of treatment discontinuation (+4 week window). If previous imaging was obtained within 4 weeks prior to the date of discontinuation, then imaging at treatment discontinuation is not mandatory. For participants who discontinue study intervention due to documented disease progression, this is the final required tumor imaging if the investigator elects not to implement iRECIST.

For participants who discontinue study intervention without documented disease progression, every effort should be made to continue monitoring disease status by tumor imaging using the same imaging schedule used while on treatment every 12 weeks (+7 days) until the start of a new anticancer treatment, disease progression, pregnancy, death, withdrawal of consent, or the end of the study, whichever occurs first.

### RECIST 1.1 Assessment of Disease

RECIST 1.1 is used as the primary measure for assessment of tumor response, date of disease progression, and as a basis for all protocol guidelines related to disease status (eg, discontinuation of study intervention). Although RECIST 1.1 references a maximum of 5 target lesions in total and 2 per organ, this protocol allows a maximum of 10 target lesions in total and 5 per organ, if clinically relevant to enable a broader sampling of tumor burden.

iRECIST Assessment of Disease

iRECIST is based on RECIST 1.1, but adapted to account for the unique tumor response seen with immunotherapeutic drugs. iRECIST will be used by the investigator to assess tumor response and progression, and make treatment decisions. When clinically stable, participants are not discontinued until progression is confirmed by the investigator, working with local radiology. This allowance to continue treatment despite initial radiologic PD takes into account the observation that some participants can have a transient tumor flare in the first few months after the start of immunotherapy, and then experience subsequent disease response.

Any participant deemed clinically unstable is discontinued from study intervention at the time when site-assessed first radiologic evidence of PD, and is not required to have repeat tumor imaging for confirmation of PD by iRECIST. If the investigator decides to continue treatment, the participant may continue to receive study intervention and the tumor assessment should be repeated 4 to 8 weeks later to confirm PD by iRECIST, per investigator assessment. If repeat imaging does not confirm PD per iRECIST, as assessed by the investigator, and the participant continues to be clinically stable, study intervention continues and follows the regular imaging schedule. If PD is confirmed, participants are discontinued from study intervention.

If a participant has confirmed radiographic progression (iCPD), study intervention is discontinued; however, if the participant is achieving a clinically meaningful benefit, an exception to continue study intervention is considered. In this case, if study intervention is continued, tumor imaging continues to be performed. A summary of imaging and treatment requirements after first radiologic evidence of progression is provided in Table 14.

TABLE 14
Imaging and Treatment after First Radiologic Evidence of Progressive Disease

| | Clinically Stable | | Clinically Unstable | |
|---|---|---|---|---|
| | Imaging | Treatment | Imaging | Treatment |
| First radiologic | Repeat | May continue | Repeat imaging | Discontinue |
| evidence of PD by | imaging at 4 | study treatment | at 4 to 8 weeks to | treatment |
| RECIST 1.1 per | to 8 weeks to | at the | confirm PD per | |
| investigator | confirm PD | assessment of | investigator's | |
| assessment | | the investigator | discretion only. | |
| | | and after the | | |
| | | participant's | | |
| | | consent | | |
| First radiologic | Repeat | May continue | Repeat imaging | Discontinue |
| evidence of PD by | imaging at 4 | study | at 4 to 8 weeks to | treatment |
| RECIST 1.1 | to 8 weeks to | intervention at | confirm PD per | |
| | confirm PD. | the | investigator's | |
| | | investigator's | discretion only. | |
| | | discretion while | | |
| | | awaiting | | |
| | | confirmatory | | |
| | | tumor imaging | | |
| | | by site by | | |
| | | iRECIST. | | |
| Repeat tumor | No additional | Discontinue | No additional | Not applicable |
| imaging confirms | imaging | treatment. | imaging | |
| PD (iCPD) by | required. | | required. | |
| iRECIST per | | | | |
| investigator | | | | |
| assessment. | | | | |
| Repeat tumor | Repeat | Continue study | Repeat imaging | Discontinue |
| imaging shows | imaging at 4 | intervention at | at 4 to 8 weeks to | treatment |
| iUPD by iRECIST | to 8 weeks to | the | confirm PD per | |
| per investigator | confirm PD. | investigator's | investigator's | |
| assessment. | May occur at | discretion. | discretion only. | |
| | next regularly | | | |
| | scheduled | | | |
| | imaging visit. | | | |
| Repeat tumor | Continue | Continue study | Continue | May restart |
| imaging shows iSD, | regularly | intervention at | regularly | study |
| iPR, or iCR by | scheduled | the | scheduled | intervention if |
| iRECIST per | imaging | investigator's | imaging | condition has |
| investigator | assessments. | discretion. | assessments. | improved and/or |
| assessment. | | | | clinically stable |
| | | | | per |
| | | | | investigator's |
| | | | | discretion. Next |
| | | | | tumor imaging |
| | | | | should occur |
| | | | | according to the |
| | | | | regular imaging |
| | | | | schedule. |

Abbreviations:
iCPD = iRECIST confirmed progressive disease;
iCR = iRECIST complete response;
iPR = iRECIST confirmed partial response;
iRECIST = modified Response Evaluation Criteria in Solid Tumors 1.1 for immune-based therapeutics;
iSD = iRECIST stable disease;
iUPD = iRECIST unconfirmed progressive disease;
PD = progressive disease;
RECIST 1.1 = Response Evaluation Criteria in Solid Tumors 1.1;
VOP = verification of progression

### Safety Assessments

Safety assessments include the collection of AEs and SAEs, monitoring of vital signs and laboratory assessments (including pregnancy tests), performance of electrocardiograms (ECGs) and physical examinations, and verification of concurrent medications.

### Adverse Events

The investigator or qualified designee assesses each subject to evaluate for potential new or worsening AEs and more frequently if clinically indicated. Assessment of AEs includes, but is not limited to, the type, incidence, severity (graded by the National Cancer Institute Common Terminology Criteria for Adverse Events [NCI CTCAE] Version 4.0), timing, seriousness, and relatedness to study drug. Adverse events that occur during the study, including baseline signs and symptoms, are recorded.

### Full Physical Examination

The investigator or qualified designee performs a complete physical exam during the Screening period. Clinically significant abnormal findings are recorded as medical history. After the first dose of study intervention, new clinically significant abnormal findings are recorded as AEs.

### Directed Physical Examination

For cycles that do not require a full physical exam, the investigator or qualified designee performs a directed physical exam as clinically indicated prior to the administration of the study intervention. New clinically significant abnormal findings are recorded as AEs.

### Vital Signs

Vital signs are measured in a semi-supine position after 5 minutes rest and include temperature, systolic and diastolic blood pressure, respiratory rate, pulse rate, and weight. Height is collected at screening only.

### Electrocardiograms

A standard 12-lead ECG is performed using local standard procedures. Clinically significant abnormal findings at Screening are recorded as medical history. Additional ECG(s) are performed on study when clinically necessary. Clinically significant findings seen on the follow-up ECGs are recorded as AEs.

### Clinical Safety Laboratory Assessments

The tests detailed in Table 15 are performed by a local laboratory. Additional tests may be performed at any time during the study as determined necessary by the investigator.

TABLE 15
Protocol-Required Safety Laboratory Assessments

| Laboratory | | | |
|---|---|---|---|
| Assessments | Parameters | | |
| Hematology | Platelet Count | RBC Indices: | WBC count with |
| | RBC Count | MCV | Differential: |
| | Hemoglobin | MCH | Neutrophils |
| | Hematocrit | % Reticulocytes | Lymphocytes |
| | | | Monocytes |
| | | | Eosinophils |
| | | | Basophils |

| | | | | |
|---|---|---|---|---|
| Chemistry | Blood Urea | Potassium | Aspartate | Total bilirubin |
| | Nitrogen (BUN) | | Aminotransferase | (and direct |
| | | | (AST)/Serum | bilirubin, if total |
| | | | Glutamic- | bilirubin is |
| | | | Oxaloacetic | elevated above |
| | | | Transaminase | the upper limit |
| | | | (SGOT) | of normal) |
| | Albumin | Bicarbonate | Chloride | Phosphorous |
| | Creatinine | Sodium | Alanine | Total Protein |
| | | | Aminotransferase | |
| | | | (ALT)/Serum | |
| | | | Glutamic- | |
| | | | Pyruvic | |
| | | | Transaminase | |
| | | | (SGPT) | |
| | Glucose | Calcium | Alkaline | TSH |
| | | | phosphatase | Total T3 |
| | | | | (or free T3) |
| | | | | Total T4 |
| | | | | (or free T4)a |

| | |
|---|---|
| Routine | Specific gravity |
| Urinalysis | pH, glucose, protein, blood, ketones, [bilirubin, urobilinogen, nitrite, |
| | leukocyte esterase] by dipstick |
| | Microscopic examination (if blood or protein is abnormal) |
| Other | Follicle-stimulating hormone and estradiol (as needed in women of |
| Screening | nonchildbearing potential only) |
| Tests | [Serum or urine] [alcohol and drug screen (to include at minimum: |
| | amphetamines, barbiturates, cocaine, opiates, cannabinoids and |
| | benzodiazepines) if applicable] |
| | [Serum or urine] β-human chorionic gonadotropin (β-hCG) pregnancy |
| | test (as needed for WOCBP) |
| | [Serology [(HIV antibody, hepatitis B surface antigen [HBsAg], and |
| | hepatitis C virus antibody)] [or specify other tests] [if applicable] |

NOTES:
aT3 and T4 are preferred; if not available, free T3 and free T4 may be tested.
Abbreviations:
β-hCG = β-human chorionic gonadotropin;
ALT = alanine transaminase;
AST = aspartate transaminase;
BUN = blood urea nitrogen;
HBsAg = hepatitis B surface antigen;
HIV = human immunodeficiency virus;
MCH = mean corpuscular hemoglobin;
MCV = mean corpuscular volume;
RBC = red blood cell;
SGOT = serum glutamic oxaloacetic transaminase;
SGPT = serum glutamic pyruvic transaminase;
TSH = thyroid stimulating hormone;
WBC = white blood cell;
WOCBP = woman/women of childbearing potential.

### Time Period and Frequency for Collecting AE, SAE, and Other Reportable Safety Event Information

All AEs, SAES, and other reportable safety events that occur after the consent form is signed but before treatment allocation/randomization must be reported by the investigator if the participant is receiving placebo run-in or other run-in treatment, if the event cause the participant to be excluded from the study, or is the result of a protocol-specified intervention, including but not limited to washout or discontinuation of usual therapy, diet, or a procedure. All AEs from the time of treatment allocation/randomization through 30 days following cessation of study intervention must be reported by the investigator.

All AEs meeting serious criteria, from the time of treatment allocation/randomization through 90 days following cessation of study intervention or 30 days following cessation of study intervention if the participant initiates new anticancer therapy, whichever is earlier, must be reported by the investigator. Additionally, any SAE brought to the attention of an investigator at any time outside of the time period specified above is reported immediately if the event is considered drug-related.

### Statistical Methods for Efficacy Analyses

Objective Response Rate (ORR)—ORR is calculated as the ratio of the number of participants reported to have achieved a confirmed CR or PR verified by BICR, divided by the number of participants included in APaT population. Participants in the APaT analysis population without ORR assessments will be counted as non-responders. A 95% exact binomial CI (based on method Clopper and Pearson,1934) is calculated for the true ORR.

Progression-Free Survival (PFS)—The non-parametric Kaplan-Meier method is used to estimate the PFS distribution. 95% CIs for the median PFS and PFS point estimates at various follow-up times from first day of study treatment will be calculated. Since disease progression is assessed periodically, PD can occur any time in the time interval between the last assessment where PD was not documented and the assessment when PD is documented. The true date of PD will be approximated by the date of the first assessment at which PD is objectively documented based on RECIST 1.1 by BICR. Death is always considered as a PFS event. Participants who do not experience a PFS event will be censored at the last disease assessment. For the analysis of PFS, if the events (PD or death) are immediately after more than one missed disease assessment, the data are censored at the last disease assessment prior to missing visits. Also, data after new anticancer therapy are censored at the last disease assessment prior to the initiation of new anticancer therapy. If a participant meets multiple criteria for censoring, the censoring criterion that occurs earliest will be applied.

Overall Survival (OS)—The non-parametric Kaplan-Meier method is used to estimate the OS distribution. 95% CIs for the median OS and OS point estimates at various follow-up times from first day of study treatment is calculated.

Duration of Response (DOR)—DOR is summarized descriptively using the non-parametric Kaplan-Meier method. Only the subset of participants who show a CR or PR are included in this analysis.

### Analysis Strategy for Key Efficacy Endpoint

Table 16 summarizes the primary analysis approach for key efficacy endpoints.

TABLE 16
Analysis Strategy for Key Efficacy Endpoints

| | | | |
|---|---|---|---|
| | | Analysis | Missing Data |
| Endpoint | Statistical Method | Population | Approach |

Primary Endpoints

| | | | |
|---|---|---|---|
| ORR per | Exact method based | APaT | Participants without |
| RECIST 1.1 | on binomial | | assessments are |
| by BICR | distribution | | considered |
| | (Clopper-Pearson | | non-responders and |
| | method) | | conservatively |
| | | | included in the |
| | | | denominator |

Key Secondary Endpoint

| | | | |
|---|---|---|---|
| PFS per | Summary statistics | APaT | Primary censoring |
| RECIST 1.1 | using Kaplan-Meier | | rule |
| by BICR | method | | Sensitivity analysis 1 |
| | | | Sensitivity analysis 2 |
| | | | (More details are |
| | | | provided in Table 15, |
| | | | Censoring Rules |
| | | | for Primary and |
| | | | Sensitivity Analyses |
| | | | of PFS) |
| OS | Summary statistics | APaT | Censored at the last |
| | using Kaplan-Meier | | known alive date |
| | method | | |
| DOR per | Summary statistics | APaT | Non-responders are |
| RECIST 1.1 | using Kaplan-Meier | | excluded from |
| by BICR | method | | analysis. |
| | | | Responders are |
| | | | censored according to |
| | | | the censoring rules |
| | | | listed in Table 15 |

a Statistical models are described in further detail in the text.
Abbreviations:
APaT = All Participants as Treated;
BICR = blinded independent central review;
DOR = duration of response;
ORR = objective response rate;
OS = overall survival;
PFS = progression-free survival;
RECIST = Response Evaluation Criteria in Solid Tumors

### Statistical Methods for Safety Analyses

Safety and tolerability are assessed by clinical review of all relevant parameters including adverse experiences and laboratory parameters. The broad AE categories consisting of the percentage of participants with any AE, a drug-related AE, a serious AE, an AE which is both drug-related and serious, and who discontinued due to an AE are summarized via point estimates with 95% CIs (Table 17).

TABLE 17
Analysis Strategy for Safety Parameters

| | Within | |
|---|---|---|
| | Group | Descriptive |
| Safety Endpoint | 95% CI | Statistics |
| Any AE | X | X |
| Any Serious AE | X | X |
| Any Drug-related AE | X | X |
| Any Serious and Drug-related AE | X | X |
| Discontinuation due to AE | X | X |
| Specific AEs, SOCs, or PDLCs | | X |
| Change from Baseline Results | | X |
| (Labs, Vital Signs) | | |

Note:
95% CIs will be calculated using the Clopper Pearson method
X = results are provided
Abbreviations:
SOC = System Organ Class;
PDLC = Pre-Defined Limit of Change

An AE is any untoward medical occurrence in a clinical study participant, temporally associated with the use of study intervention, whether or not considered related to the study intervention. An AE can therefore be any unfavorable and unintended sign (including an abnormal laboratory finding), symptom, or disease (new or exacerbated) temporally associated with the use of the drug. The following are included as AEs:
Any abnormal laboratory test results (hematology, clinical chemistry, or urinalysis) or other safety assessments (eg, ECG, radiological scans, vital signs measurements), including those that worsen from baseline, or are considered clinically significant in the medical and scientific judgment of the investigator.
Exacerbation of a chronic or intermittent pre-existing condition including either an increase in frequency and/or intensity of the condition.
New conditions detected or diagnosed after study intervention administration even though it may have been present before the start of the study.
Signs, symptoms, or the clinical sequelae of a suspected drug-drug interaction.
Signs, symptoms, or the clinical sequelae of a suspected overdose of either study intervention or a concomitant medication.
Worsening of signs and symptoms of malignancy during the study is reported as an AE. Disease progression assessed by measurement of malignant lesions on radiographs or other methods are not be reported as an AE, unless the event results in hospitalization or death.
   The following events do not meet the AE definition for purposes of this study:
Medical or surgical procedure (eg, endoscopy, appendectomy): the condition that leads to the procedure is the AE.
Situations in which an untoward medical occurrence did not occur (social and/or convenience admission to a hospital).
Anticipated day-to-day fluctuations of pre-existing disease(s) or condition(s) present or detected at the start of the study that do not worsen.
Surgery planned prior to informed consent to treat a pre-existing condition that has not worsened.

If an event is not an AE per definition above, then it cannot be an SAE even if serious conditions are met. An SAE is defined as any untoward medical occurrence that, at any dose:
Results in death
Is life-threatening. The term “life-threatening” in the definition of “serious” refers to an event in which the participant was at risk of death at the time of the event. It does not refer to an event, which hypothetically might have caused death, if it were more severe.
Requires inpatient hospitalization or prolongation of existing hospitalization. Hospitalization is defined as an inpatient admission, regardless of length of stay, even if the hospitalization is a precautionary measure for continued observation. Hospitalization for an elective procedure to treat a pre-existing condition that has not worsened is not an SAE. A pre-existing condition is a clinical condition that is diagnosed prior to the use of an MSD product and is documented in the participant's medical history.
Results in persistent or significant disability/incapacity. The term disability means a substantial disruption of a person's ability to conduct normal life functions. This definition is not intended to include experiences of relatively minor medical significance such as uncomplicated headache, nausea, vomiting, diarrhea, influenza, and accidental trauma (eg, sprained ankle) that may interfere with or prevent everyday life functions but do not constitute a substantial disruption.
Is a congenital anomaly/birth defect in offspring of participant taking the product regardless of time to diagnosis.

Medical or scientific judgment is exercised in deciding whether SAE reporting is appropriate in other situations such as important medical events that may not be immediately life-threatening or result in death or hospitalization but may jeopardize the participant or may require medical or surgical intervention to prevent 1 of the other outcomes listed in the above definition. These events are usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

### Demographics and Baseline Characteristics

The number and percentage of subjects screened, allocated, the primary reasons for screening failure, and the primary reasons for discontinuation are displayed. Demographic variables (e.g., age, gender), baseline characteristics, primary and secondary diagnoses, and prior and concomitant therapies is summarized either by descriptive statistics or categorical tables for all enrolled subjects.

### Subgroup Analyses

To determine whether the response rate is consistent across various subgroups, the estimate of the response rate (with a nominal 95% CI) for the primary endpoint is estimated within each category of the following classification variables:
Age category (<65 vs. ≥65 years)
Sex (female vs. male)
Race (white vs. non-white)
Disease stage (III vs. IVM1a vs. IVM1b vs IVM1c)
Brain metastasis (yes vs. no)
ECOG status (0 vs. 1)
PD-L1 status (positive vs. negative)
BRAF wild type versus BRAF mutant (no prior treatment) versus BRAF mutant (prior treatment)
   A Forest plot is produced, which provides the estimated point estimates and CIs for the treatment effect across the categories of subgroups listed above. Any specified subgroups that have less than 10 participants are excluded from analysis.

### REFERENCES

1. Sharpe, A. H, Wherry, E. J., Ahmed R., and Freeman G. J. The function of programmed cell death 1 and its ligands in regulating autoimmunity and infection. Nature Immunology (2007); 8:239-245.
2. Dong H et al. Tumor-associated B7-H1 promotes T-cell apoptosis: a potential mechanism of immune evasion. Nat Med. 2002 Aug;8(8):793-800.
3. Yang et al. PD-1 interaction contributes to the functional suppression of T-cell responses to human uveal melanoma cells in vitro. Invest Ophthalmol Vis Sci. 2008 Jun;49(6 (2008): 49: 2518-2525.
4. Ghebeh et al. The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198.
5. Hamanishi J et al. Programmed cell death 1 ligand 1 and tumor-infiltrating CD8+ T lymphocytes are prognostic factors of human ovarian cancer. Proceeding of the National Academy of Sciences (2007): 104: 3360-3365.
6. Thompson RH et al. Significance of B7-H1 overexpression in kidney cancer. Clinical genitourin Cancer (2006): 5: 206-211.
7. Nomi, T. Sho, M., Akahori, T., et al. Clinical significance and therapeutic potential of the programmed death- 1 ligand/programmed death-1 pathway in human pancreatic cancer. Clinical Cancer Research (2007);13:2151-2157.
8. Ohigashi Y et al. Clinical significance of programmed death-1 ligand-1 and programmed death-1 ligand 2 expression in human esophageal cancer. Clin. Cancer Research (2005): 11: 2947-2953.
9. Inman et al. PD-L1 (B7-H1) expression by urothelial carcinoma of the bladder and BCG-induced granulomata: associations with localized stage progression. Cancer (2007): 109: 1499-1505.
10. Shimauchi T et al. Augmented expression of programmed death-1 in both neoplasmatic and nonneoplastic CD4+ T-cells in adult T-cell Leukemia/Lymphoma. Int. J. Cancer (2007): 121:2585-2590.
11. Gao et al. Overexpression of PD-L1 significantly associates with tumor aggressiveness and postoperative recurrence in human hepatocellular carcinoma. Clinical Cancer Research (2009) 15: 971-979.
12. Nakanishi J. Overexpression of B7-H1 (PD-L1) significantly associates with tumor grade and postoperative prognosis in human urothelial cancers. Cancer Immunol Immunother. (2007) 56: 1173- 1182.
13. Hino et al. Tumor cell expression of programmed cell death-1 is a prognostic factor for malignant melanoma. Cancer (2010): 00: 1-9.
14. Ghebeh H. Foxp3+ tregs and B7-H1+/PD-1+ T lymphocytes co-infiltrate the tumor tissues of high-risk breast cancer patients: implication for immunotherapy. BMC Cancer. 2008 Feb 23;8:57.
15. Ahmadzadeh M et al. Tumor antigen-specific CD8 T cells infiltrating the tumor express high levels of PD-1 and are functionally impaired. Blood (2009) 114: 1537-1544.
16. Thompson RH et al. PD-1 is expressed by tumor infiltrating cells and is associated with poor outcome for patients with renal carcinoma. Clinical Cancer Research (2007) 15: 1757-1761.

All references cited herein are incorporated by reference to the same extent as if each individual publication, database entry (e.g. Genbank sequences or GeneID entries), patent application, or patent, was specifically and individually indicated to be incorporated by reference. U.S. provisional application 62/755,756 is incorporated by reference in its entirety. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1-68. (canceled)

69. A method for treating cancer in a patient comprising administering to the patient 700 or 800 mg of an anti-LAG3 antibody via intravenous infusion, wherein the anti-LAG3 antibody comprises: (a) a light chain comprising CDRs of SEQ ID NOs: 26, 27 and 28 and (b) a heavy chain comprising CDRs of SEQ ID NOs: 29, 30 and 31.

70. The method of claim 69, wherein the patient is administered 800 mg of the anti-LAG3 antibody.

71. The method of claim 70, wherein the patient is administered the anti-LAG3 antibody on Day 1 once every three weeks.

72. The method of claim 71, wherein the anti-LAG3 antibody consists of two heavy chains and two light chains, and wherein the heavy chain comprises a heavy chain variable region comprising SEQ ID NO:25 and the light chain comprises a light chain variable region comprising SEQ ID NO: 24.

73. The method of claim 71, wherein the anti-LAG3 antibody consists of two heavy chains and two light chains, and wherein the heavy chain comprises SEQ ID NO:23 and the light chain comprises SEQ ID NO:22.

74. The method of claim 71, wherein the anti-LAG3 antibody is an Ab6 variant.

75. The method of claim 73, wherein the anti-LAG3 antibody is co-administered with an anti-PD-1 antibody or anti-PD-L1 antibody, or antigen binding fragment thereof.

76. The method of claim 73, wherein the anti-LAG3 antibody is co-formulated with an anti-PD-1 antibody or anti-PD-L1 antibody or antigen binding fragment thereof.

77. The method of claim 76, wherein the anti-PD-1 antibody, or antigen binding fragment thereof specifically binds to human PD-1 and blocks the binding of human PD-L1 and human PD-L2 to human PD-1.

78. The method of claim 77, wherein the anti-PD-1 antibody, or antigen binding fragment thereof comprises: (a) a light chain comprising CDRs of SEQ ID NOs: 1, 2 and 3 and (b) a heavy chain comprising CDRs of SEQ ID NOs: 6, 7 and 8.

79. The method of claim 78, wherein the anti-PD-1 antibody consists of two heavy chains and two light chains, and wherein the heavy chain comprises a heavy chain variable region comprising SEQ ID NO: 9 and the light chain comprises a light chain variable region comprising SEQ ID NO: 4.

80. The method of claim 79, wherein the anti-PD-1 antibody consists of two heavy chains and two light chains, and wherein the heavy chain comprises SEQ ID NO: 10 and the light chain comprises SEQ ID NO: 5.

81. The method of claim 77, wherein the anti-PD-1 antibody is pembrolizumab.

82. The method of claim 77, wherein the anti-PD-1 antibody is a pembrolizumab variant.

83. The method of claim 81, wherein the pembrolizumab is administered at 200 mg via intravenous infusion on Day 1 once every three weeks.

84. The method of claim 82, wherein the pembrolizumab variant is administered at 200 mg via intravenous infusion on Day 1 once every three weeks.

85. The method of claim 81, wherein the pembrolizumab is administered at 400 mg via intravenous infusion on Day 1 once every six weeks.

86. The method of claim 77, wherein the anti-PD-1 antibody consists of two heavy chains and two light chains, and wherein the heavy chain comprises a heavy chain variable region comprising SEQ ID NO: 9 and the light chain comprises a light chain variable region comprising SEQ ID NO: 4; and the anti-LAG3 antibody consists of two heavy chains and two light chains, and wherein the heavy chain comprises a heavy chain variable region comprising SEQ ID NO: 25 and the light chain comprises a light chain variable region comprising SEQ ID NO: 24.

87. The method of claim 77, wherein the anti-PD-1 antibody consists of two heavy chains and two light chains, and wherein the heavy chain comprises SEQ ID NO: 10 and the light chain comprises SEQ ID NO: 5; and the anti-LAG3 antibody consists of two heavy chains and two light chains, and wherein the heavy chain comprises SEQ ID NO: 23 and the light chain comprises SEQ ID NO: 22.

88. The method of claim 87, wherein the anti-PD-1 antibody is administered at 200 mg via intravenous infusion on Day 1 once every three weeks, and the anti-LAG3 antibody is administered at 800 mg via intravenous infusion on Day 1 once every three weeks.

89. The method of claim 87, wherein the anti-PD-1 antibody is administered at 400 mg via intravenous infusion on Day 1 once every six weeks, and the anti-LAG3 antibody is administered at 800 mg via intravenous infusion on Day 1 once every three weeks.

90. The method of claim 88, wherein 200 mg of anti-PD-1 antibody is co-formulated with 800 mg anti-LAG3 antibody.

91. The method of any one of claims 73-74, 87-88 and 90, wherein the cancer is non-microsatellite instability-high (non-MSI-H) or proficient mismatch repair (pMMR) colorectal cancer.

92. The method of any one of claims 73-74, 87-88 and 90, wherein the cancer is selected from the group consisting of: gastric cancer, adenocarcinoma of the stomach and/or gastric-esophageal junction, esophagus cancer, renal cell carcinoma, melanoma, non-small cell lung cancer, small cell lung cancer, classical Hodgkin lymphoma (cHL), diffuse large B-cell lymphoma (DLBCL), indolent non-Hodgkin lymphoma (iNHL).

93. The method of claim 87, wherein the patient has not been previously treated with anti-PD-1 or anti-PD-L1 therapy or is confirmed progressive while receiving prior anti-PD-1 or anti-PD-L1 therapy.

94. The method of claim 91, wherein the tumor tissue section of the patient has a Combined Positive Score for PD-L1 expression ≥1%.

95. The method of claim 92, wherein the tumor tissue section of the patient has a Combined Positive Score for PD-L1 expression of ≥1% or ≥10%.

96. The method of claim 94, wherein the PD-L1 expression is measured by the PD-L1 IHC 22C3 pharmDx assay.

97. A pharmaceutical composition comprising 200 mg pembrolizumab or pembrolizumab variant, and 800 mg of Ab6 or Ab6 variant, and a pharmaceutically acceptable excipient.

98. A pharmaceutical composition comprising 200 mg pembrolizumab, and 800 mg of an anti-LAG3 antibody consisting of two heavy chains and two light chains, and wherein the heavy chain comprises a heavy chain variable region comprising SEQ ID NO:25 and the light chain comprises a light chain variable region comprising SEQ ID NO: 24, and a pharmaceutically acceptable excipient.

99. The pharmaceutical composition of claim 98, wherein the anti-LAG3 antibody consists of two heavy chains and two light chains, wherein the heavy chain comprises SEQ ID NO:23 and the light chain comprises SEQ ID NO:22.

100. A method for treating gastric cancer in a patient comprising administering to the patient an anti-LAG3 antibody and an anti-PD-1 antibody, wherein a tumor tissue section from the gastric tumor of the patient is PD-L1 expression positive.

101. The method of claim 100, wherein the gastric cancer is adenocarcinoma of the stomach and/or gastric-esophageal junction adenocarcinoma.

102. A method for treating a patient with head and neck squamous cell carcinoma comprising administering to the patient an anti-LAG3 antibody and an anti-PD-1 antibody, wherein a tumor tissue section from the head and neck tumor of the patient is PD-L1 expression positive.

103. A method for treating a patient with non-microsatellite instability-high (non-MSI-H) or proficient mismatch repair (pMMR) colorectal cancer comprising administering to the patient an anti-LAG3 antibody and an anti-PD-1 antibody, wherein a tumor tissue section from the colorectal tumor of the patient is PD-L1 expression positive, and the % LAG3 positive cells or CPS-like % LAG3 positive cells is ≥1%.


SEQUENCE LISTING (33 sequences)

SEQ ID NO: 1 - protein, 15 aa, Artificial Sequence
  Synthetic polypeptide
RASKGVSTSGYSYLH

SEQ ID NO: 2 - protein, 7 aa, Artificial Sequence
  Synthetic polypeptide
LASYLES

SEQ ID NO: 3 - protein, 9 aa, Artificial Sequence
  Synthetic polypeptide
QHSRDLPLT

SEQ ID NO: 4 - protein, 111 aa, Artificial Sequence [sequence omitted]

SEQ ID NO: 5 - protein, 218 aa, Artificial Sequence
  Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Synthetic polypeptide
NYYMY

SEQ ID NO: 7 - protein, 17 aa, Artificial Sequence
  Synthetic polypeptide
GINPSNGGTNFNEKFKN

SEQ ID NO: 8 - protein, 11 aa, Artificial Sequence
  Synthetic polypeptide
RDYRFDMGFDY

SEQ ID NO: 9 - protein, 120 aa, Artificial Sequence [sequence omitted]

SEQ ID NO: 10 - protein, 447 aa, Artificial Sequence
  Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence
  Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 440 aa, Artificial Sequence
  Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 17 aa, Artificial Sequence
  Synthetic polypeptide
KSSQSLLHTSTRKNYLA

SEQ ID NO: 14 - protein, 7 aa, Artificial Sequence
  Synthetic polypeptide
WASTRES

SEQ ID NO: 15 - protein, 8 aa, Artificial Sequence
  Synthetic polypeptide
KQSYDVVT

SEQ ID NO: 16 - protein, 112 aa, Artificial Sequence
  Synthetic polypeptide
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 17 - protein, 5 aa, Artificial Sequence
  Synthetic polypeptide
SYWIH

SEQ ID NO: 18 - protein, 10 aa, Artificial Sequence
  Synthetic polypeptide
GYTFTSYWIH

SEQ ID NO: 19 - protein, 17 aa, Artificial Sequence
  Synthetic polypeptide
YINPSSGYHEYNQKFID

SEQ ID NO: 20 - protein, 13 aa, Artificial Sequence
  Synthetic polypeptide
SGWLIHGDYYFDF

SEQ ID NO: 21 - protein, 122 aa, Artificial Sequence
  Synthetic polypeptide
  MISC_FEATURE (1)..(1): wherein X = Q or pE (pyro-glutamate)
XVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS

SEQ ID NO: 22 - protein, 218 aa, Artificial Sequence
  Synthetic polypeptide
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 446 aa, Artificial Sequence
  Synthetic polypeptide
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 111 aa, Artificial Sequence
  Synthetic polypeptide
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 25 - protein, 119 aa, Artificial Sequence
  Synthetic polypeptide
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 26 - protein, 15 aa, Artificial Sequence
  Synthetic polypeptide
KASQSLDYEGDSDMN

SEQ ID NO: 27 - protein, 7 aa, Artificial Sequence
  Synthetic polypeptide
GASNLES

SEQ ID NO: 28 - protein, 9 aa, Artificial Sequence
  Synthetic polypeptide
QQSTEDPRT

SEQ ID NO: 29 - protein, 5 aa, Artificial Sequence
  Synthetic polypeptide
DYNVD

SEQ ID NO: 30 - protein, 17 aa, Artificial Sequence
  Synthetic polypeptide
DINPNDGGTIYAQKFQE

SEQ ID NO: 31 - protein, 10 aa, Artificial Sequence
  Synthetic polypeptide
NYRWFGAMDH

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL